(19) United States
(12) Patent Application Publication
(10) Pub. No.: US 2025/0270323 A1
(43) Pub. Date: Aug. 28, 2025

(54) HHLA2 BINDING AGENTS WITH NOVEL ACTIVITY

(71) Applicant: NextPoint Therapeutics, Inc., Cambridge, MA (US)

(72) Inventors: Scott Chappel, Milton, MA (US); Detlev Biniszkiewicz, Boston, MA (US); Bijan Etemad-Gilbertson, Jamaica Plain, MA (US); Nadthakarn Boland, Hanover, NH (US); Nels Nielson, Hopkinton, NH (US); Bianka Prinz, Lebanon, NH (US)

(21) Appl. No.: 18/263,313

(22) PCT Filed: Jan. 28, 2022
(86) PCT No.: PCT/US22/14423

§ 371 Date: Jul. 27, 2023

Related U.S. Application Data
(60) Provisional application No. 63/142,832, filed on Jan. 28, 2021.

(51) Int. Cl. C07K 16/28

(52) U.S. Cl. CPC C07K 16/2827; C07K 2317/76; C07K 2317/92

(57) ABSTRACT

The present disclosure pertains to HHLA2 binding agents with novel activity and uses thereof.

### CROSS REFERENCE TO RELATED APPLICATIONS

This application claims priority to U.S. Provisional Patent Application No. 63/142,832, filed on Jan. 28, 2021, which is incorporated herein by reference in its entirety.

### BACKGROUND

Although immunotherapies have been investigated for many diseases and disorders, including cancer, functional limitations have been encountered that still need to be addressed. The immune system includes a tightly controlled by a network of costimulatory and co-inhibitory ligands and receptors. Immune checkpoints negatively regulate immune response progression based on complex interactions. Currently available immune checkpoint inhibitors can modulate immune responses in some patients, but immune checkpoint expression and interactions with natural binding partners can vary between patients.

Therefore, a need exists for the development of new therapeutic modalities optimized to target immune checkpoint pathways.

### SUMMARY

HERV-H LTR-Associating 2 (HHLA2), a B7 gene family member, is broadly expressed in a variety of tumors and antigen presenting cells. HHLA2 is known to interact with both inhibitory and stimulatory receptors to regulate T-cell functions. Killer-cell immunoglobulin-like receptor (KIR) proteins include either two (KIR2D) or three (KIR3D) immunoglobulin-like extracellular domains, and KIR3DL3 is an inhibitory HHLA2 receptor found on T cells and NK cells. HHLA2 binding to KIR3DL3 has been shown to inhibit the immune response of activated T cells and the cytotoxic activity of NK cells.

In contrast, transmembrane and immunoglobulin domain containing 2 (TMIGD2) is an activating receptor for HHLA2. Concomitant with T cell receptor (TCR) signaling, TMIGD2 on naïve T cells interacts with HHLA2 and co-stimulates T cell activation via a pathway involving AKT phosphorylation. With repetitive T cell activation, expression of stimulatory receptor TMIGD2 is gradually lost, allowing expression of the inhibitory receptor KIR3DL3 to become dominant.

While HHLA2 binding agents have been investigated as immunotherapies, the present disclosure encompasses, inter alia, the discovery of HHLA2 binding agents described herein with novel activity that result in: (i) inhibition of HHLA2 binding to KIR3DL3; and/or (ii) enhancement of HHLA2 binding to TMIGD2. In some embodiments, HHLA2 binding agents are capable of: (i) inhibiting HHLA2 binding to KIR3DL3; and (ii) enhancing HHLA2 binding to TMIGD2. In some embodiments, HHLA2 binding agents described herein cause allosteric changes in HHLA2, thereby resulting in conformational changes in HHLA2 that enhance HHLA2 binding to TMIGD2. In some embodiments, HHLA2 binding agents described herein directly compete with at least one binding site for TMIGD2 on HHLA2. In some embodiments, a HHLA2 binding agent described herein enhances an early stage immune response and/or a later stage immune response. In some embodiments, a HHLA2 binding agent described herein enhances HHLA2 binding to TMIGD2 in naïve immune effector cells. In some embodiments, a HHLA2 binding agent described herein blocks HHLA2 binding to KIR3DL3 in exhausted immune effector cells. In some embodiments, exhausted immune effector cells comprise or express certain cell surface markers, such as PD-1, CTLA-4, LAG-3, TIM-3, 2B4/CD244/SLAMF4, CD160, and/or TIGIT.

Accordingly, the present disclosure provides several examples of such HHLA2 binding agents that are particularly useful for treating a variety of cancers, including solid tumors, such as non-small cell lung cancer (NSCLC), renal cell carcinoma (RCC), cholangiocarcinoma, or breast cancer, and hematological tumors, as well as modulating an immune response in a subject.

In some embodiments, an HHLA2 binding agent described herein is used for tumor targeting of at least one cytotoxic agent. In some embodiments, an HHLA2 binding agent described herein is administered or co-formulated with a cytotoxic agent. In some embodiments, an HHLA2 binding agent described herein is used for delivery of at least one radionuclide to a tumor (e.g., a tumor described herein). In some embodiments, an HHLA2 binding agent described herein is administered or co-formulated with a radionuclide. In some embodiments, an HHLA2 binding agent described herein is used for targeting a tumor (e.g., a tumor described herein) in combination with a monoclonal antibody that binds Fc receptor (FcR), thereby mediating antibody dependent cellular cytotoxicity (ADCC). In some embodiments, an HHLA2 binding agent described herein is administered or co-formulated with a monoclonal antibody that binds FcR.

In one aspect, the disclosure provides HHLA2 binding agent capable of: (i) inhibiting HHLA2 binding to KIR3DL3; and/or (ii) enhancing HHLA2 binding to TMIGD2.

In some embodiments, an HHLA2 binding agent is or comprises an antibody or antigen-binding fragment thereof, a small molecule, a polypeptide, or an aptamer.

In some embodiments, an antibody or antigen-binding fragment thereof is or comprises: (i) a chimeric antibody, a human antibody, or a humanized antibody, or antigen-binding fragment thereof, (ii) a monospecific antibody or a bispecific antibody, or antigen-binding fragment thereof, and/or (iii) a monoclonal antibody, or antigen-binding fragment thereof. In some embodiments, an antigen-binding fragment comprises an scFv, Fab, Fab′, F(ab′)2, Fc, nanobody, or camelid antibody. In some embodiments, an antibody or antigen-binding fragment thereof is or comprises: (i) a heavy chain constant region chosen from IgG1, IgG2, IgG3, or IgG4, and/or (ii) a light chain constant region chosen from the light chain constant regions of kappa or lambda.

In some embodiments, an antibody or antigen-binding fragment thereof is or comprises: (a) a heavy chain variable region (VH) comprising one, two, or three VH CDR sequences each with at least about 90% identity to a VH CDR of Table 1; and/or (b) a light chain variable region (VL) comprising one, two, or three VL CDR sequences each with at least about 90% identity to a VL CDR of Table 1. In some embodiments, an antibody or antigen-binding fragment thereof is or comprises: (a) a VH comprising one, two, or three VH CDR sequences each with at least about 91%, 92%, 93%, 94%, 95%, 96%, 97%, 98%, 99%, 99.5% or higher identity to a VH CDR of Table 1; and/or (b) a VL comprising one, two, or three VL CDR sequences each with at least about 91%, 92%, 93%, 94%, 95%, 96%, 97%, 98%, 99%, 99.5% or higher identity to a VL CDR of Table 1. In some embodiments, an antibody or antigen-binding fragment thereof is or comprises: (a) a VH comprising one, two, or three VH CDR sequences each comprising or consisting of a VH CDR of Table 1; and/or (b) a VL comprising one, two, or three VL CDR sequences each comprising or consisting of a VL CDR of Table 1.

In some embodiments, an antibody or antigen-binding fragment thereof is or comprises: (a) a VH with at least about 90% or more identity to a VH of Table 1; and/or (b) a VL with at least about 90% or more identity to a VL of Table 1. In some embodiments, an antibody or antigen-binding fragment thereof is or comprises: (a) a VH with at least about 95%, 96%, 97%, 98%, 99%, 99.5% or higher identity to a VH of Table 1; and/or (b) a VL with at least about 95%, 96%, 97%, 98%, 99%, 99.5% or higher identity to a VL of Table 1. In some embodiments, an antibody or antigen-binding fragment thereof is or comprises: (a) a VH comprising or consisting of a VH of Table 1; and/or (b) a VL comprising or consisting of a VL of Table 1.

In some embodiments, an antibody or antigen-binding fragment thereof is or comprises: (a) a heavy chain with at least about 90% or more identity to a heavy chain of Table 1; and/or (b) a light chain with at least about 90% or more identity to a light chain of Table 1. In some embodiments, an antibody or antigen-binding fragment thereof is or comprises: (a) a heavy chain with at least about 95%, 96%, 97%, 98%, 99%, 99.5% or higher identity to a heavy chain of Table 1; and/or (b) a light chain with at least about 95%, 96%, 97%, 98%, 99%, 99.5% or higher identity to a light chain of Table 1. In some embodiments, an antibody or antigen-binding fragment thereof is or comprises: (a) a heavy chain comprising or consisting of a heavy chain of Table 1; and/or (b) a light chain comprising or consisting of a light chain of Table 1.

In another aspect, the disclosure provides agents that bind and/or compete for binding with the same epitope on HHLA2 as an HHLA2 binding agent of any aspect or embodiment described herein.

In some embodiments, an HHLA2 binding agent enhances HHLA2 binding to TMIGD2 in naïve immune effector cells. In some embodiments, an HHLA2 binding agent blocks HHLA2 binding to KIR3DL3 in exhausted immune effector cells. In some embodiments, immune effector cells comprise or are T cells and/or NK cells. In some embodiments, T cells comprise or are CD4+ T cells and/or CD8+ T cells.

In some embodiments, an HHLA2 binding agent binds HHLA2 with a KD of about 5 nM or less. In some embodiments, an HHLA2 binding agent binds HHLA2 with a KD of about 15 nM or less. In some embodiments, an HHLA2 binding agent binds human HHLA2 with an affinity of at least about 50-fold to about 800-fold over background. In some embodiments, an HHLA2 binding agent enhances HHLA2 binding to TMIGD2 at a ratio of greater than about 2.

In another aspect, the disclosure provides pharmaceutical compositions comprising at least one HHLA2 binding agent of any aspect or embodiment described herein, and a pharmaceutically acceptable carrier.

In another aspect, the disclosure provides methods of treating a subject having a disease, disorder, or condition comprising: administering a therapeutically effective amount of at least one HHLA2 binding agent of any aspect or embodiment described herein, or a pharmaceutical composition of any aspect or embodiment described herein.

In another aspect, the disclosure provides methods of modulating an immune response in a subject comprising: administering a therapeutically effective amount of at least one HHLA2 binding agent of any aspect or embodiment described herein, or a pharmaceutical composition of any aspect or embodiment described herein.

In some embodiments, a subject has or is at risk of developing a cancer. In some embodiments, a subject has a solid tumor or a hematological cancer. In some embodiments, a solid tumor is or comprises one or more of: a renal cancer, a bone cancer, a skin cancer, a breast cancer, a cervical cancer, a colorectal cancer, an endometrial cancer, a lung cancer, an ovarian cancer, a liver cancer, cholangiocarcinoma, or a thyroid cancer. In some embodiments, a hematological cancer comprises or is a leukemia or lymphoma. In some embodiments, a leukemia comprises or is acute lymphocytic leukemia, acute myeloid leukemia, chronic myeloid leukemia, acute lymphoblastic leukemia, chronic lymphocytic leukemia, chronic leukemia, or acute leukemia. In some embodiments, a lymphoma comprises or is Hodgkin lymphoma (HL), non-Hodgkin's lymphoma, lymphocytic lymphoma, or diffuse large B cell lymphoma (DLBCL).

In some embodiments, a disease, disorder, or condition is associated with aberrant HHLA2 expression.

In some embodiments, an HHLA2 binding agent is administered parenterally. In some embodiments, parenteral administration is or comprises subcutaneous, intravenous, intramuscular, or intrasternal injection or infusion.

In some embodiments, an HHLA2 binding agent is administered in combination with a second agent.

In another aspect, the disclosure provides nucleic acids encoding at least one HHLA2 binding agent of any aspect or embodiment described herein, or an antigen-binding fragment thereof.

In another aspect, the disclosure provides expression vectors comprising at least one nucleic acid of any aspect or embodiment described herein.

In another aspect, the disclosure provides host cells comprising or expressing at least one HHLA2 binding agent of any aspect or embodiment described herein, comprising at least one nucleic acid of aspect or embodiment described herein, or comprising at least one expression vector of aspect or embodiment described herein.

In another aspect, the disclosure provides methods of making an HHLA2 binding agent, comprising: (i) culturing a host cell comprising at least one nucleic acid of any aspect or embodiment described herein or at least one expression vector of any aspect or embodiment described herein under conditions suitable for expression of the HHLA2 binding agent, and (ii) recovering the HHLA2 binding agent.

In another aspect, the disclosure provides methods of detecting the presence or level of an HHLA2 polypeptide in a sample comprising: detecting an HHLA2 polypeptide in a sample using at least one HHLA2 binding agent of any aspect or embodiment described herein.

In another aspect, the disclosure provides kits comprising at least one HHLA2 binding agent of any aspect or embodiment described herein, and instructions for use and/or administration.

In some embodiments, an HHLA2 binding agent forms a complex with an HHLA2 polypeptide. In some embodiments, a complex is detected by an assay comprising an enzyme linked immunosorbent assay (ELISA), radioimmune assay (RIA), and/or Western blot. In some embodiments, a HHLLA2 binding agent is directly labeled.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The Figures described below, which together make up the Drawing, are for illustration purposes only, not for limitation.

FIGS. 1A-1B are schematics showing an alignment of heavy chain variable domains (FIG. 1A) and light chain variable domains (FIG. 1A) of exemplary anti-HHLA2 antibodies Ab-60638 and Ab-60665.

FIGS. 2A-2B are graphs showing binding affinity of exemplary anti-HHLA2 antibodies Ab-60638 (FIG. 2A) and Ab-60665 (FIG. 2B) for recombinant HHLA2-Fc using the Octet system from ForteBio.

FIGS. 3A-3C are graphs of flow cytometry histograms showing binding of exemplary anti-HHLA2 antibodies Ab-60638 (FIG. 3A), Ab-60665 (FIG. 3B), and Ab-65885/Ab-65886/Ab-65887/Ab-65889/Ab-65890 (FIG. 3C) to 300.19 cells over-expressing human HHLA2 relative to an isotope control.

FIGS. 4A-4B are graphs of flow cytometry data showing the ability of exemplary anti-HHLA2 antibodies Ab-60638 and Ab-60665 to block binding of human HHLA2-Fc to 300.19 cells over-expressing human KIR3DL3 (FIG. 4A) and enhance binding of human TMIGD2-Fc to 300.19 cells over-expressing human HHLA2 (FIG. 4B), both relative to an isotype control.

FIGS. 5A-5B are graphs of flow cytometry data showing the ability of exemplary anti-HHLA2 antibodies Ab-65885, Ab-65886, Ab-65887, Ab-65889, and Ab-65890 to block binding of human HHLA2-Fc to 300.19 cells over-expressing human KIR3DL3 (FIG. 5A) and enhance binding of human TMIGD2-Fc to 300.19 cells over-expressing human HHLA2 (FIG. 5B), both relative to an isotype control.

FIG. 6 is a bar graph showing that exemplary anti-HHLA2 antibodies Ab-65885, Ab-65886, Ab-65887, Ab-65889 and Ab-65890 modulate activity of HHLA2-expressing CHO cells on adjacent TMIGD2-expressing Jurkat cells using a luciferase reporter in Jurkat cells; the assay shows that not only do these five exemplary antibodies not block the HHLA2-mediated signaling via TMIGD2, but they enhance HHLA2-mediated signaling via TMIGD2.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an agent” means one agent or more than one agent.

About: As used herein, the term “about,” as applied to one or more values of interest, refers to a value that is similar to a stated reference value. In some embodiments, the term “about” refers to a range of values that fall within 25%, 20%, 19%, 18%, 17%, 16%, 15%1, 4%1, 3%1, 12%, 1%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Affinity matured: As used herein, the term “affinity matured” refers to an antibody with one or more alterations in one or more CDRs thereof, which result in an improvement in affinity of an antibody for an antigen, compared to a parent antibody that does not possess those one or more alterations. In some embodiments, affinity matured antibodies will have nanomolar or even picomolar affinities for a target antigen. Affinity matured antibodies may be produced by any of a variety of procedures known in the art. Affinity maturation by VH and VL domain shuffling is described in Marks et al., BioTechnology 10:779-783 (1992). Random mutagenesis of CDR and/or framework residues is described in: Barbas et al. Proc. Nat. Acad. Sci. U.S.A 91:3809-3813 (1994); Schier et al., Gene 169: 147-155 (1995); Yelton et al., J. Immunol. 155: 1994-2004 (1995); Jackson et al., J. Immunol. 154(7):3310-9 (1995); and Hawkins et al., J. Mol. Biol. 226:889-896 (1992).

Agent: As used herein, the term “agent” refers to a biological entity and/or compound including, for example, an antibody or antigen-binding fragment thereof, an organic molecule (e.g., a small molecule), a peptide (e.g., a fusion protein), an aptamer, a nucleic acid, a chimeric antigen receptor, a glycoprotein, a saccharide, a lipid, a growth factor, an enzyme, a synthetic molecule, a carbohydrate, a lipid, a hormone, a polymer, or a derivative, variation, complex, or any combination thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product. In some instances, again as will be clear from context, the term may be used to refer to one or more entities that is man-made in that it is designed, engineered, and/or produced through human action and/or is not found in nature. In some embodiments, an agent may be utilized in isolated or pure form. In some embodiments, an agent may be utilized in crude form. In some embodiments, agents are provided as collections or libraries, which may be screened to identify or characterize active agents within them. An agent may bind any cell moiety, such as a receptor, an antigenic determinant, or other binding site present on a target or target cell. Various agents are useful in the compositions and methods described herein.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch,” connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another so that the dimers are connected to one another and a tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present disclosure include glycosylated Fe domains, such as Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen) or produced by recombinant engineering, chemical synthesis, or other artificial system or methodology. In some embodiments, an antibody is polyclonal. In some embodiments, an antibody is monoclonal. In some embodiments, an antibody has constant region sequences characteristic of mouse, rabbit, primate, or human antibodies. In some embodiments, antibody sequence elements are humanized, primatized, or chimeric as is known in the art. Moreover, the term “antibody,” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bispecific or multi-specific antibodies (e.g., Zybodies®, etc); and/or antibody fragments (preferably antibody fragments that exhibit desired antigen-binding activity). An antibody described herein can be an immunoglobulin, heavy chain antibody, light chain antibody, LRR-based antibody, or other protein scaffold with antibody-like properties, as well as any other immunological binding moiety known in the art, e.g., a Fab, Fab′, Fab′2, Fab2, Fab3, F(ab′)2, Fd, Fv, Feb, scFv, SMIP, antibody, diabody, triabody, tetrabody, minibody, maxibody, tandab, DVD, BiTe, TandAb, or any combination thereof. The subunit structures and three-dimensional configurations of different classes of antibodies are known in the art. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc), or other pendant group (e.g., poly-ethylene glycol, etc.).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, or chimeric, as is known in the art. In many embodiments, the term “antibody agent” is used to refer to one or more of the art-known or developed constructs or formats for utilizing antibody structural and functional features in alternative presentation. For example, an antibody agent utilized in accordance with the present invention is in a format including, but not limited to, intact IgA, IgG, IgE or IgM antibodies; bi-specific or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; or KALBITOR®s. In some embodiments, an antibody lacks a covalent modification (e.g., attachment of a glycan) that it would have if produced naturally. In some embodiments, an antibody contains a covalent modification (e.g., attachment of a glycan, a payload, e.g., a detectable moiety, a therapeutic moiety, or a catalytic moiety), or other pendant group (e.g., poly-ethylene glycol). In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments, an included CDR substantially identical to a reference CDR in that it is either identical in sequence or contains between 1-5 amino acid substitutions as compared with the reference CDR. In some embodiments an included CDR is substantially identical to a reference CDR by at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR by at least 96%, 96%, 97%, 98%, 99%, or 100% sequence identity with the reference CDR. In some embodiments, an included CDR is substantially identical to a reference CDR in that at least one amino acid within the included CDR is deleted, added, or substituted as compared with the reference CDR, but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR, but the included CDR has an amino acid sequence that is otherwise identical to the reference CDR. In some embodiments, an included CDR is substantially identical to a reference CDR in that at least one amino acid within the included CDR is substituted as compared with the reference CDR, but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR, but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain that is homologous or largely homologous to an immunoglobulin-binding domain.

Antibody heavy chain: As used herein, the term “antibody heavy chain” refers to the larger of the two types of polypeptide chains present in all antibodies in their naturally occurring conformations.

Antibody light chain: As used herein, the term “antibody light chain” refers to the smaller of the two types of polypeptide chains present in all antibodies in their naturally occurring conformations.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequence or a partial nucleotide sequence encoding a protein that elicits an immune response encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell, or a biological fluid.

Antigen-binding fragment: As used herein, the term “antigen-binding fragment” refers to a portion of an intact antibody that binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2; diabodies; linear antibodies; single-chain antibody molecules (e.g. scFv, VHH, camelid, or VH or VL domains only); or multispecific antibodies formed from antibody fragments. In some embodiments, the antigen-binding fragments of the antibodies described herein are scFvs. In some embodiments, the antigen-binding fragments of the antibodies described herein are VHH domains only. As with full antibody molecules, antigen-binding fragments may be monospecific or multispecific (e.g., bispecific). A multispecific antigen-binding fragment of an antibody may comprise at least two different variable domains, wherein each variable domain is capable of specifically binding to a separate antigen or to a different epitope of the same antigen. An antigen-binding fragment may be produced by any means. For example, in some embodiments, an antigen-binding fragment is enzymatically or chemically produced by fragmentation of an intact antibody or antibody agent. Alternatively, in some embodiments, an antigen-binding fragment is recombinantly produced. In some embodiments, an antigen-binding fragment is wholly or partially synthetically produced. In some embodiments, an antigen-binding fragment has a length of at least about 50, 60, 70, 80, 90, 100, 110, 120, 130, 140, 150, 160, 170, 180, 190, or 200 amino acids or more.

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by theory, ADCC is typically understood to involve Fc receptor (FcR)-bearing effector cells recognizing and subsequently killing antibody-coated target cells (e.g., cells that express on their surface specific antigens to which an antibody is bound). Effector cells that mediate ADCC include immune cells including, but not limited to, natural killer (NK) cells, macrophage, neutrophils, and eosinophils.

Aptamer: As used herein, the term “aptamer” refers to a macromolecule composed of nucleic acid (e.g., RNA, DNA) that binds tightly to a specific molecular target (e.g., an umbrella topology glycan). A particular aptamer may be described by a linear nucleotide sequence and is typically about 15-60 nucleotides in length. Without wishing to be bound by any theory, it is contemplated that the chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers typically have very high affinities for their targets (e.g., affinities in the picomolar to low nanomolar range for proteins). In many embodiments, aptamers are chemically stable and can be boiled or frozen without loss of activity. Because they are synthetic molecules, aptamers are amenable to a variety of modifications, which can optimize their function for particular applications. For example, aptamers can be modified to dramatically reduce their sensitivity to degradation by enzymes in the blood for use in in vivo applications. In addition, aptamers can be modified to alter their biodistribution or plasma residence time.

Associated. Two events or entities are “associated” with one another, as that term is used herein, if the presence, level, degree, type and/or form of one is correlated with that of the other. For example, a particular entity (e.g., polypeptide, genetic signature, metabolite, or microbe) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities physically associated with one another are covalently linked to one another. In some embodiments, two or more entities physically associated with one another are not covalently linked to one another but are non-covalently associated, for example, by means of hydrogen bonds, van der Waals interaction, hydrophobic interactions, magnetism, and combinations thereof.

Binding: As used herein, the term “binding” refers to a non-covalent association between or among two or more entities. “Direct” binding involves physical contact between entities or moieties. Indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Cancer: As used herein, the terms “cancer,” “malignancy,” “neoplasm,” “tumor,” and “carcinoma,” refer to cells that exhibit relatively abnormal, uncontrolled, and/or autonomous growth, so that they exhibit an aberrant growth phenotype characterized by a significant loss of control of cell proliferation. Cancer cells can spread locally or through the bloodstream and lymphatic system to other parts of the body. In some embodiments, a tumor is or comprises cells that are precancerous (e.g., benign), malignant, pre-metastatic, metastatic, and/or non-metastatic. In some embodiments, cancer is or comprises a solid tumor. In some embodiments cancer is or comprises a hematologic tumor. Examples of various cancers are described herein and include, but are not limited to, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers, such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers, nervous system cancers, or benign lesions, such as papillomas, as well as several other types including those as described elsewhere herein.

Carrier: as used herein, “carrier” refers to a diluent, adjuvant, excipient, and/or vehicle with which a composition is administered. In some exemplary embodiments, carriers include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

CDR: As used herein, “CDR” refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. The exact definitional CDR boundaries and lengths are subject to different classification and numbering systems. Certain systems have been established in the art for defining CDR boundaries (e.g., Kabat, IMGT, Chothia, or a combination thereof). CDRs may therefore be referred to by Kabat, Chothia, IMGT, or any other boundary definitions known in the art. Despite differing boundaries, each of these systems has some degree of overlap in what constitutes the “hypervariable regions” within the variable sequences. CDR definitions according to these systems may therefore differ in length and boundary areas with respect to the adjacent framework region (see, e.g., Kabat et al., in “Sequences of Proteins of Immunological Interest,” 5th Edition, U.S. Department of Health and Human Services, 1992; Chothia et al. (1987) J. Mol. Biol. 196, 901; and MacCallum et al., J. Mol. Biol. (1996) 262, 732, each of which is incorporated by reference in its entirety). Those skilled in the art appreciate the differences between and among these systems and are capable of understanding CDR boundaries to the extent required to understand and to practice the claims and disclosure herein.

Chemotherapeutic Agent: The term “chemotherapeutic agent,” as used herein has its art-understood meaning referring to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some embodiments, a chemotherapeutic agent may be or comprise one or more of Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g. DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, or combinations thereof. In some embodiments, a chemotherapeutic agent may be utilized in the context of an antibody-drug conjugate. In some embodiments, a chemotherapeutic agent is an antibody-drug conjugate comprising: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and/or lorvotuzumab mertansine.

Chimeric antibody: as used herein, refers to an antibody whose amino acid sequence includes VH and VL region sequences that are found in a first species and constant region sequences that are found in a second species, different from the first species. In many embodiments, a chimeric antibody has murine VH and VL regions linked to human constant regions. In some embodiments, an antibody with human VH and VL regions linked to non-human constant regions (e.g., a mouse constant region) is referred to as a “reverse chimeric antibody.”

Composition: Those skilled in the art will appreciate that the term “composition” may be used to refer to a discrete physical entity that comprises one or more specified components. In general, unless otherwise specified, a composition may be of any form—e.g., gas, gel, liquid, or solid.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Conservative sequence modifications: As used herein, the term “conservative sequence modifications” refers to amino acid modifications that do not significantly affect or alter the binding characteristics of an antibody or antigen-binding fragment thereof containing the amino acid sequence. Such conservative modifications include amino acid substitutions, additions, and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Combination therapy: The term “combination therapy”, as used herein, refers to those situations in which two or more different therapeutic agents are administered in overlapping regimens so that the subject is simultaneously exposed to both agents. When used in combination therapy, two or more different therapeutic agents may be administered simultaneously or separately. This administration in combination can include simultaneous administration of the two or more therapeutic agents in the same dosage form, simultaneous administration in separate dosage forms, and separate administration. That is, two or more therapeutic agents can be formulated together in the same dosage form and administered simultaneously. Alternatively, two or more therapeutic agents can be simultaneously administered, wherein the agents are present in separate formulations. In another alternative, a first therapeutic agent can be administered followed by one or more additional therapeutic agents. In the separate administration protocol, two or more therapeutic agents may be administered a few minutes apart, or a few hours apart, a few days apart, or a few weeks apart. In some embodiments, two or more therapeutic agents may be administered within hours (e.g., less than about 1 hour, about 2 hours, about 3 hours, about 4 hours, or about 5 hours) apart.

Effective amount: As used herein, an “effective amount” refers to a dose that is adequate to prevent or treat at least one sign and/or symptom of a disease, disorder or condition (e.g., cancer) in an individual. Amounts effective for a therapeutic or prophylactic use will depend on, for example, the stage and severity of the disease, disorder or condition being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations. For purposes of the disclosure, the amount or dose of a therapeutic agent (e.g., at least one HHLA2 binding agent described herein) administered should be sufficient to effect a therapeutic or prophylactic response in a subject over a reasonable time frame (e.g., reduction or other lessening of severity or duration of at least one sign or symptom). For example, the dose should be sufficient to detect, treat, or prevent cancer in a period of from about 2 hours or longer, e.g., about 12 to about 24 or more hours, from the time of administration. In some embodiments, the time period is even longer. The dose will be determined by the efficacy of one or more particular therapeutic agents and condition of a subject (e.g., a human) as well as body weight of a subject (e.g., a human) to be treated.

Encoding: As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA or mRNA) or a defined sequence of amino acids and biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments, an engineered polynucleotide comprises or is a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, engineered antibodies or antigen-binding fragments thereof (e.g., engineered monoclonal antibodies or antigen-binding fragments thereof) include VH and/or VL region sequences from a reference antibody raised in a non-human species (e.g., a mouse) and modifications in those sequences relative to a reference antibody intended to render them more “human-like” or more similar to human germline variable sequences.

As is common practice and is understood by those in the art, progeny of an engineered polynucleotide or cell are typically still referred to as “engineered” even though the actual manipulation was performed on a prior entity.

Epitope: As used herein, the term “epitope” refers to any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Expression: As used herein, the term “expression” of a nucleic acid sequence refers to generation of any gene product from a nucleic acid sequence (e.g., a nucleic acid sequence encoding an anti-HHLA2 antibody or antigen-binding fragment thereof described herein). In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fragment: As used herein, the term “fragment” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, an antigen-binding fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., amino acids) as found in a whole antibody. In some embodiments, an antigen-binding fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in a whole antibody. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide.

Framework region: as used herein, the term “framework region” refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression and/or inducible expression).

Homology: As used herein, the term “homology” refers to the overall relatedness between polymeric molecules, e.g., between nucleic acids (e.g., DNA and/or RNA) and/or between polypeptides. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences.

Host cell: as used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term host cell as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., or Streptomyces spp.), mycobacteria cells, fungal cells, yeast cells (e.g., S. cerevisiae, S. pombe, P. pastoris, or P. methanolica), plant cells, insect cells (e.g., SF-9, SF-21, baculovirus-infected insect cells, or Trichoplusia ni), non-human animal cells, human cells, or cell fusions (e.g., hybridomas or quadromas). In some embodiments, the cell comprises or is a human, monkey, ape, hamster, rat, or mouse cell. In some embodiments, the cell is a eukaryotic cell chosen from: CHO (e.g., CHO K1, DXB-11 CHO, Veggie-CHO), COS (e.g., COS-7), retinal cell, Vero, CVI, kidney (e.g., HEK293, 293 EBNA, MSR 293, MDCK, HaK, BHK), HeLa, HepG2, W138, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, or a cell line derived from an aforementioned cell. In some embodiments, a cell comprises one or more viral genes.

Human antibody: as used herein, the term “human antibody” refers to antibodies having variable and constant regions generated (or assembled) from human immunoglobulin sequences. Antibodies or antigen-binding fragments thereof may be considered “human” even though their amino acid sequences include residues or elements not encoded by human germline immunoglobulin sequences (e.g., sequence variations that may have been introduced by random or site-specific mutagenesis in vitro or by somatic mutation in vivo), such as in one or more CDRs and in particular CDR3.

Humanized: as used herein, the term “humanized” refers to antibodies or antigen-binding fragments thereof whose amino acid sequence includes VH and/or VL region sequences from a reference antibody raised in a non-human species (e.g., a mouse), but also includes modifications in those sequences relative to the reference antibody intended to render them more “human-like” or more similar to human germline variable sequences. In some embodiments, a humanized antibody or antigen-binding fragment thereof is one that immunospecifically binds to an antigen of interest and has a FR region with substantially the amino acid sequence of a human antibody and a CDR with substantially the amino acid sequence of a non-human antibody. A humanized antibody comprises substantially all of at least one, and typically two, variable domains (Fab, Fab′, F(ab′)2, FabC, Fv) in which all or substantially all of the CDR regions correspond to a non-human immunoglobulin (e.g., a donor immunoglobulin) and all or substantially all of the framework regions correspond to a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a CH1, hinge, CH2, CH3, and, optionally, a CH4 region of a heavy chain constant region. In some embodiments, a humanized antibody only contains a humanized VL region. In some embodiments, a humanized antibody only contains a humanized VH region. In some certain embodiments, a humanized antibody contains humanized VH and VL regions.

Identity: As used herein, the term “identity” refers to the subunit sequence identity between two polymeric molecules, particularly between two amino acid molecules, such as between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half of the positions (e.g., five positions in a polymer of 10 amino acids in length) in two sequences are identical, the two sequences are 50% identical; if 90% of the positions (e.g., nine positions in a polymer of 10 amino acids in length) are identical, the two amino acids sequences are 90% identical.

Immune cell: As used herein, the term “immune cell,” refers to a cell that is involved in an immune response, e.g., promotion of an immune response. Examples of immune cells include, but are not limited to, T cells, natural killer (NK) cells, macrophages, monocytes, dendritic cells, neutrophils, eosinophils, mast cells, platelets, large granular lymphocytes, Langerhans' cells, or B-lymphocytes.

Immune checkpoint: As used herein, the term “immune checkpoint” refers to a group of molecules on the cell surface of CD4+ and/or CD8+ T cells as well as NK cells that fine-tune immune responses by down-modulating or inhibiting an anti-tumor immune response. Immune checkpoint proteins are well-known in the art and include, without limitation, HHLA2, KIR family receptors, CTLA-4, PD-1, VISTA, B7-H2, B7-H3, PD-L1, B7-H4, B7-H6, ICOS, HVEM, PD-L2, CD160, gp49B, PIR-B, TIM-1, TIM-3, TIM-4, LAG-3, GITR, 4-IBB, OX-40, BTLA, SIRPα, CD47, CD48, 2B4 (CD244), B7.1, B7.2, ILT-2, ILT-4, TIGIT, CD226, CD155, CD112. butyrophilins, and A2aR. In some embodiments, NK cells comprise TIGIT, CD226, and/or CD96. The term further encompasses biologically active protein fragment, as well as nucleic acids encoding full-length immune checkpoint proteins and biologically active protein fragments thereof. In some embodiment, the term further encompasses any fragment according to homology descriptions provided herein.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen. In some embodiments, an immune cell response can include proliferation of an immune effector cell (e.g., a T cell), cytokine production by an immune effector cell (e.g., a T cell), and/or release of cytotoxic granules comprising perforin and/or granzymes by an immune effector cell (e.g., a T cell).

Immunoglobulin: As used herein, the term “immunoglobulin” or “Ig,” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function, but may serve as an antigen receptor. IgE is an immunoglobulin that mediates immediate hypersensitivity by causing release of mediators from mast cells and basophils upon exposure to allergen.

“Improve,” “increase”, “inhibit” or “reduce”: As used herein, the terms “improve,” “increase,” “inhibit,” “reduce,” or grammatical equivalents thereof, indicate values that are relative to a baseline or other reference measurement. In some embodiments, an appropriate reference measurement is or comprises a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement is or comprises a measurement in comparable system known or expected to respond in a particular way, in presence of the relevant agent or treatment.

Isolated: As used herein, the term “isolated” refers to something altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

K_{D}: as used herein, the term “K_{D}” refers to the dissociation constant of a binding agent (e.g., an antibody or antigen-binding fragment thereof) from a complex with its partner (e.g., the epitope to which the antibody or antigen-binding fragment thereof binds). The term “K_{D},” as used herein, equals K_{off} divided by Kₒₙ.

K_{off}: as used herein, the term “K_{off}” refers to the off rate constant for dissociation of a binding agent (e.g., an antibody or antigen-binding fragment thereof) from a complex with its partner (e.g., the epitope to which the antibody or antigen-binding fragment thereof binds).

Kₒₙ: as used herein, the term “Kₒₙ” refers to the on rate constant for association of a binding agent (e.g., an antibody or antigen-binding fragment thereof) with its partner (e.g., the epitope to which the antibody or antigen-binding fragment thereof binds).

Modulating: As used herein the term “modulating,” refers to mediating a detectable increase or decrease in a level of a response and/or change in nature of a response in a subject compared with a level and/or nature of a response in a subject without a treatment or an untreated subject. The term encompasses perturbing and/or affecting a native signal or response thereby mediating a beneficial therapeutic response in a subject, preferably, a human.

Monoclonal Antibody: A “monoclonal antibody” or “mAb” refers to an antibody obtained from a population of substantially homogeneous antibodies, such that individual antibodies of the population are identical and/or bind the same epitope, except for possible variant antibodies (e.g., containing naturally occurring mutations or arising during production of a monoclonal), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, a nucleic acid may comprise a backbone that comprises one or more phosphorothioate or 5′-N-phosphoramidite linkages and/or one or more peptide bonds, e.g., as in a peptide nucleic acid. In some embodiments, a nucleic acid comprises one or more, or all, natural residues (e.g., adenine, cytosine, deoxyadenosine, deoxycytidine, deoxyguanosine, deoxythymidine, guanine, thymine, and/or uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, or combinations thereof). In some embodiments, a non-natural residue comprises one or more modified sugars (e.g., 2′-fluororibose, ribose, 2′-deoxyribose, arabinose, and/or hexose) as compared to those in natural residues. In some embodiments, a nucleic acid has a nucleotide sequence that encodes a functional gene product, such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: As used herein, the term “operably linked” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Pharmaceutically acceptable: As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Polypeptide: As used herein, the terms “polypeptide” or “protein,” as used interchangeably herein, refer to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. A polypeptide may comprise or consist of natural amino acids, non-natural amino acids, or both. A polypeptide may comprise or consist of only natural amino acids or only non-natural amino acids. A polypeptide may comprise D-amino acids, L-amino acids, or both. A polypeptide may include one or more pendant groups or other modifications, e.g., modifying or attached to one or more amino acid side chains at the N-terminus, at the C-terminus, or both. In some embodiments, such pendant groups or modifications are chosen from acetylation, amidation, lipidation, methylation, or pegylation, including combinations thereof. A polypeptide may be cyclic and/or may comprise a cyclic portion. In some embodiments, a polypeptide is not cyclic and/or does not comprise any cyclic portion. In some embodiments, a polypeptide is linear. A polypeptide may be or comprise a stapled polypeptide. The term “polypeptide” may be appended to a name of a reference polypeptide, activity, or structure; in such instances, it is used herein to refer to polypeptides that share the relevant activity or structure and thus can be considered members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class. For example, a member polypeptide may have an overall degree of sequence homology or identity with a reference polypeptide that is at least about 30-40% and is often about 50%, 60%, 70%, 80%, 90%, 91%, 92%, 93%, 94%, 95%, 96%, 97%, 98%, 99% or more and/or includes at least one region (e.g., a conserved region that may be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. A useful polypeptide may comprise or consist of a fragment of a parent polypeptide. A useful polypeptide may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent) so that the polypeptide of interest is a derivative of its parent polypeptide.

Single chain antibodies: As used herein, the term “single chain antibodies” refers to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; and Skerra et al. (1988) Science 242:1038-1041.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell, polypeptides isolated from a recombinant, combinatorial human polypeptide library (see, e.g., Hoogenboom, TIB Tech 15:62, 1997; Azzazy Clin. Biochem. 35:425, 2002; Gavilondo BioTechniques 29:128, 2002; Hoogenboom Immunology Today 21:371, 2000), antibodies isolated from an animal (e.g., a mouse) that is transgenic for human immunoglobulin genes (see, e.g., Taylor Nuc. Acids Res. 20:6287, 1992; Little Immunology Today 12:364, 2000; Kellermann Curr. Opin. Biotechnol 13:593, 2002; Murphy Proc. Natl Acad Sci USA 111:5153, 2104) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant antibody polypeptide is comprised of sequences found in the germline of a source organism of interest (e.g., human, mouse, etc.). In some embodiments, a recombinant antibody has an amino acid sequence that resulted from mutagenesis (e.g., in vitro or in vivo, for example in a transgenic animal), so that the amino acid sequences of the VH and VL regions of the recombinant antibodies are sequences that, while originating from and related to germline VH and VL sequences, do not naturally exist within the germline antibody repertoire in vivo.

Small molecule: As used herein, the term “small molecule” refers to a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, or glycolipid. In some embodiments, a small molecule is not a lipid. In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent. Those of ordinary skill in the art, reading the present disclosure, will appreciate that certain small molecule compounds may be provided and/or utilized in any of a variety of forms such as, for example, crystal forms, salt forms, protected forms, pro-drug forms, ester forms, isomeric forms (e.g., optical and/or structural isomers), or isotopic forms. Those of skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form. Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms. Those of skill in the art will appreciate that certain small molecule compounds have structures that permit isotopic substitution (e.g., ²H or ³H for H; ¹¹C, ¹³C or ¹⁴C for 12C; ¹³N or ¹⁵N for 14N; ¹⁷O or ¹⁸O for 16O; ³⁶Cl for XXC; ¹⁸F for XXF; 131I for XXXI; etc). In some embodiments, such a small molecule may be utilized in accordance with the present disclosure in one or more isotopically modified forms, or mixtures thereof. In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form. In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound is considered a different form of the compound than a racemic mixture of the compound; a particular salt of a small molecule compound is considered a different form from another salt form of the compound; a preparation that contains only a form of the compound that contains one conformational isomer ((Z) or (E)) of a double bond is considered to a different form of the compound from one that contains the other conformational isomer ((E) or (Z)) of the double bond; or a preparation in which one or more atoms is a different isotope than is present in a reference preparation is considered to be a different form.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments, a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition. In some embodiments, a susceptible subject is predisposed to and/or shows an increased risk (as compared to the average risk observed in a reference subject or population) of developing a disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantial identity: As used herein, the term “substantial identity” refers to a comparison between amino acid or nucleic acid sequences. As will be appreciated by those of ordinary skill in the art, two sequences are generally considered to be substantially identical if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with and/or displays one or more symptoms of a disease, disorder, and/or condition.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by, for example, a HHLA2 binding agent described herein.

Therapeutic: As used herein, the term “therapeutic” refers to a treatment and/or prophylaxis. A therapeutic effect is obtained, for example, by suppression, remission, or eradication of a disease state.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent can be an agent that, when administered to a subject, can prevent an undesired side effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. A therapeutic agent includes, but is not limited to, at least one HHLA2 binding agent as described herein.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, and/or the target cell or tissue. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose. In some embodiments, multiple unit doses are required to deliver a therapeutically effective amount.

Treat: As used herein, the terms “treat,” “treatment,” or “treating” refer to partial or complete alleviation, amelioration, delay of onset of, inhibition, prevention, relief, and/or reduction in incidence and/or severity of one or more symptoms or features of a disease, disorder, and/or condition. In some embodiments, treatment is administered to a subject who does not exhibit signs or features of a disease, disorder, and/or condition (e.g., may be prophylactic). In some embodiments, treatment is administered to a subject who exhibits only early or mild signs or features of the disease, disorder, and/or condition, for example for the purpose of decreasing the risk of developing pathology associated with the disease, disorder, and/or condition. In some embodiments, treatment is administered to a subject who exhibits established, severe, and/or late-stage signs of the disease, disorder, or condition. In some embodiments, treating comprises administering at least one HHLA2 binding agent described herein to a subject.

Tumor: As used herein, the term “tumor” refers to an abnormal growth of cells or tissue. A tumor may comprise cells that are precancerous (e.g., benign), malignant, pre-metastatic, metastatic, and/or non-metastatic. In some embodiments, a tumor is associated with or is a manifestation of a cancer. In some embodiments, a tumor is a disperse tumor or a liquid tumor. In some embodiments, a tumor is a solid tumor.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

The present disclosure, among other things, provides HHLA2 binding agents that result in: (i) inhibition of HHLA2 binding to KIR3DL3; and/or (ii) enhancement of HHLA2 binding to TMIGD2. In some embodiments, HHLA2 binding agents are capable of: (i) inhibiting HHLA2 binding to KIR3DL3; and (ii) enhancing HHLA2 binding to TMIGD2. HHLA2 is a B7 family member that modulates NK cell and T cell functions. HHLA2 is broadly expressed in a variety of tumors and antigen presenting cells and has been implicated as both an activating and inhibitory ligand for NK cells and T cells. HHLA2 is a specific ligand for TMIGD2 and the interaction of HHLA2 and TMIGD2 selectively stimulates T cell proliferation and cytokine production. HHLA2 also binds KIR3DL3, a receptor on T cells and NK cells, resulting in inhibition of T cell and NK cell activation. The present disclosure provides HHLA2 binding agents for treating a variety of cancers, including solid tumors and hematological tumors, and/or modulating an immune response in a subject.

### HHLA2 Binding Agents

The present disclosure, among other things, provides HHLA2 binding agents. In some embodiments, an HHLA2 binding agent described herein exhibits the ability to: (i) inhibit HHLA2 binding to one or more receptors that inhibit an immune response (e.g. TMIGD2), and/or (ii) enhance HHLA2 binding to one or more receptors that promote an immune response (e.g. TMIGD2). Accordingly, HHLA2 binding agents described herein are particularly useful for treating a variety of cancers, including solid and hematological tumors, as well as modulating an immune response in a subject.

The terms “HHLA2” or “human endogenous retrovirus-H long terminal repeat-associating protein 2” refers to a member of the B7 family. HHLA2 is also known as HERV-H LTR-associating 2, B7y, B7H7, or B7-H7. HHLA2 protein has limited expression in normal human tissues, but is widely expressed in human cancers. HHLA2 is a membrane protein with three Ig-like domains (IgV-IgC-IgV), whereas other members of the B7 family generally have only two Ig domains (IgV-IgC). HHLA2 in normal human tissues is expressed in the epithelium of kidney, gut, gallbladder, and breast as well as placental trophoblast cells. In the immune system, HHLA2 is constitutively expressed on human monocytes and macrophages. HHLA2 regulates human T cell functions including, for example, T cell proliferation and cytokine production. HHLA2 is expressed in higher levels in a wide range of human cancers from the colorectal, renal, lung, pancreas, ovary, and prostate. HHLA2 is also expressed in human cancers of thyroid, melanoma, liver, bladder, colon, kidney, breast, and esophagus.

The term “HHLA2” includes fragments, variants (e.g., allelic variants), and derivatives thereof. Representative human HHLA2 cDNA and human HHLA2 protein sequences are publicly available from the National Center for Biotechnology Information (NCBI). Human HHLA2 variants include variant 1 (NM_007072.3 and NP_009003.1, which represents the longest transcript and encodes the longest isoform a), variant 2 (NM_001282556.1 and NP_001269485.1, which represents the use of an alternate promoter and differs in the 5′ UTR, compared to variant 1), variant 3 (NM_001282557.1 and NP_001269486.1, which represents the use of an alternate promoter and differs in the 5′ UTR, compared to variant 1), variant 4 (NM_001282558.1 and NP_001269487.1, which encodes isoform b, represents the use of an alternate promoter, differs in the 5′ UTR and lacks an alternate in-frame exon in the 3′ coding region, compared to variant 1, resulting a shorter isoform than isoform a), and variant 5 (NM_001282559.1 and NP_001269488.1, which encodes isoform c, represents the use of an alternate promoter, and has multiple differences compared to variant 2, resulting in a distinct 5′ UTR and causing translation initiation at an alternate start codon, compared to variant 1, resulting in a distinct N-terminus and a shorter isoform than isoform a).

In some embodiments, binding of an HHLA2 binding agent described herein to HHLA2 is assessed using an assay, such as bio-layer interferometry (BLI), immunohistochemical (IHC), Western blot, intercellular flow, ELISA, surface plasmon resonance (SPR), isothermal titration calorimetry (ITC), or any other known method in the art). In some embodiments, an HHLA2 binding agent described herein binds to HHLA2 with a K_{D} of about 20 nM to about 0.1 nM, e.g., about 10 nM to about 0.1 nM, e.g., about 5 nM to about 0.5 nM. In some embodiments, an HHLA2 binding agent described herein binds to HHLA2 with a K_{D} of about 20 nM or less, about 15 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, or about 0.1 nM or less.

In some embodiments, HHLA2 binding agents described herein inhibit HHLA2 binding to KIR3DL3. The terms “KIR3DL3” or “Killer cell immunoglobulin-like receptor 3DL3,” as used herein, refer to a member of the Killer cell immunoglobulin-like receptor transmembrane glycoprotein family expressed by NK cells and T cells. KIR3DL3 is also known as KIRC1, CD158Z, KIR3DL7, and KIR44. The killer cell immunoglobulin-like receptor (KIR) genes are polymorphic and highly homologous genes found in a cluster on chromosome 19q13.4 within the 1 Mb leukocyte receptor complex (LRC). The gene content of the KIR gene cluster varies among haplotypes, although several “framework” genes are found in all haplotypes (KIR3DL3, KIR3DL1, KIR3DL4, and KIR3DL2). The KIR proteins are classified by the number of extracellular immunoglobulin domains (2D or 3D) and by whether they have a long (L) or short (S) cytoplasmic domain. KIR proteins with the long cytoplasmic domain transduce inhibitory signals upon ligand binding via an immune tyrosine-based inhibitory motif (ITIM), while KIR proteins with the short cytoplasmic domain lack the ITIM motif and instead associate with the TYRO protein tyrosine kinase binding protein to transduce activating signals. The ligands for several KIR proteins are subsets of HLA class I molecules; thus, KIR proteins are thought to play an important role in regulation of the immune response. The KIR3DL3 protein has an N-terminal signal sequence, 3 Ig domains, a transmembrane region lacking a positively charged residue, and a long cytoplasmic tail containing an ITIM. KIR3DL3 lacks the stalk region found in other KIRs.

The term “KIR3DL3” includes fragments, variants (e.g., allelic variants), and derivatives thereof. Representative human KIR3DL3 cDNA and human KIR3DL3 polypeptide sequences are publicly available from NCBL For example, at least one human KIR3DL3 isoform is known: human KIR3DL3 (NM_153443.4) encoded by the transcript (NP_703144.3). Nucleic acid and polypeptide sequences of KIR3DL3 orthologs in organisms other than humans are also known including, but not limited to, chimpanzee KIR3DL3 (XM_003316679.3 and XP_003316727.3), Rhesus monkey KIR3DL3 (NM_001104552.2 and NP_001098022.1), mouse KIR3DL3 (NM_001310690.1 and NP_001297619.1, NM_177749.4 and NP_808417.2, NM_177748.2 and NP_808416.1), and rat KIR3DL3 (NM_181479.2 and NP_852144.1).

In some embodiments, inhibition of HHLA2 binding to KIR3DL3 by an HHLA2 binding agent described herein is assessed using an assay, such as a cell binding competition assay (e.g., an assay of soluble HHLA2 binding to KIR3DL3-expressing cells (e.g., KIR3DL3-expressing 300.19 mouse pre-B leukemic cells)), surface plasmon resonance (SPR), or any other known method in the art. In some embodiments, an HHLA2 binding agent described herein inhibits binding of HHLA2 to KIR3DL3 at a ratio of about 0.8 to about 0.0, e.g., relative to an isotope control. In some embodiments, an HHLA2 binding agent described herein inhibits binding of HHLA2 to KIR3DL3 at a ratio of about 0.8, about 0.7, about 0.6, about 0.5, about 0.4, about 0.3, about 0.2, about 0.1, or about 0.0, e.g., relative to an isotope control. In some embodiments, an HHLA2 binding agent described herein abolishes binding of HHLA2 to KIR3DL3.

In some embodiments, HHLA2 binding agents described herein enhance HHLA2 binding to TMIGD2. The terms “TMIGD2” or “transmembrane and immunoglobulin domain containing 2 (TMIGD2),” as used herein, refer to a membrane protein having an extracellular IgV-like domain, a transmembrane region, and a proline-rich cytoplasmic domain with two tyrosine signaling motifs. TMIGD2 is constitutively expressed on naïve T cells and natural killer (NK) cells, but not on T regulatory cells or B cells. TMIGD2 expression is slowly lost with repetitive stimulation of T cells. Consistent with this, TMIGD2 is expressed on only about half of memory T cells, and TMIGD2-negative T cells have a terminally-differentiated, senescent phenotype. TMIGD2 is also expressed in endothelial and epithelial cells and functions to reduce cell migration and promote capillary tube formation during angiogenesis.

The term “TMIGD2” is intended to include fragments, variants (e.g., allelic variants), and derivatives thereof. Representative human TMIGD2 cDNA and human TMIGD2 protein sequences are publicly available from NCBI. Human TMIGD2 isoforms include isoform 1 (NM_144615.2 and NP 653216.2), isoform 2 (NM_001169126.1 and NP 001162597.1; which uses an alternate in-frame splice site in the 3′ coding region, compared to variant 1, resulting a shorter isoform, compared to isoform 1), and isoform 3 (NM_001308232.1 and NP 001295161.1, which lacks an alternate in-frame exon in the 5′ coding region compared to variant 1, resulting a shorter isoform, compared to isoform 1). Nucleic acid and polypeptide sequences of TMIGD2 orthologs in organisms other than humans are also known including, for example, chimpanzee TMIGD2 (XM_009434393.2 and XP_009432668.2, and XM_OO1 138228.4 and XP_001138228.3), and cattle TMIGD2 (XM_005208980.3 and XP 005209037.1, XM_005208979.3 and XP_005209036.1, and XM_002688933.5 and XP_002688979.1).

In some embodiments, enhancement of HHLA2 binding to TMIGD2 by an HHLA2 binding agent described herein is assessed using an assay, such as a cell binding competition assay (e.g., an assay of soluble TMIGD2 binding to HHLA2-expressing cells (e.g., HHLA2-expressing 300.19 mouse pre-B leukemic cells)), or any other known method in the art. In some embodiments, an HHLA2 binding agent described herein enhances binding of HHLA2 to TMIGD2 at a ratio of about 2.0 to about 8.0, e.g., relative to an isotope control. In some embodiments, an HHLA2 binding agent described herein enhances binding of HHLA2 to TMIGD2 at a ratio of about 2.0, about 2.5, about 3.0, about 3.5, about 4.0, about 4.5, about 5.0, about 5.5, about 6.0, about 6.5, about 7.0, about 7.5, about 8.0, or greater, e.g., relative to an isotope control.

In some embodiments, an HHLA2 binding agent is or comprises an antibody or antigen-binding fragment thereof. In some embodiments, an HHLA2 binding agent is or comprises an organic molecule (e.g., a small molecule). In some embodiments, an HHLA2 binding agent is or comprises a polypeptide (e.g., a fusion polypeptide). In some embodiments, an HHLA2 binding agent is or comprises an aptamer. In some embodiments, an HHLA2 binding agent is or comprises a nucleic acid. In some embodiments, an HHLA2 binding agent is or comprises a chimeric antigen receptor (e.g., a CAR comprising an anti-HHLA2 antigen-binding fragment described herein, such as an scFv).

### Anti-HHLA2 Antibodies and Fragments Thereof

The present disclosure, among other things, provides anti-HHLA2 antibodies or antigen-binding fragments thereof. In some embodiments, an anti-HHLA2 antibody or antigen-binding fragment thereof described herein binds specifically to an epitope on HHLA2. In some embodiments, an anti-HHLA2 antibody or antigen-binding fragment thereof described herein can be or comprise an immunoglobulin, heavy chain antibody, light chain antibody, or other protein scaffold with antibody-like properties, as well as other immunological binding moiety known in the art, including a Fab fragment, a Fab′ fragment, a F(ab′)2 fragment, a Fv fragment, a disulfide-bonded Fv fragment, a scFv fragment, a diabody, a triabody, a tetrabody, a minibody, a maxibody, a tandab, BiTe, or any combination thereof. In some embodiments, an anti-HHLA2 antibody or antigen-binding fragment thereof described herein inhibits binding of HHLA2 to KIR3DL3. In some embodiments, an anti-HHLA2 antibody or antigen-binding fragment thereof described herein enhances binding of HHLA2 to TIMGD2.

In some embodiments, an anti-HHLA2 antibody or antigen-binding fragment thereof described herein comprises or is a monoclonal antibody. In some embodiments, an anti-HHLA2 antibody or antigen-binding fragment thereof described herein comprises or is a full length antibody, e.g., comprising an immunoglobulin Fc region. In some embodiments, an anti-HHLA2 antibody or antigen-binding fragment thereof described herein comprises or is a multispecific antibody, e.g., comprising a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, an anti-HHLA2 antibody or antigen-binding fragment thereof described herein comprises or is a bispecific antibody molecule. In some embodiments, an anti-HHLA2 antibody or antigen-binding fragment thereof described herein is or has been affinity matured.

An anti-HHLA2 antibody or antigen-binding fragment thereof can include a heavy chain variable domain sequence (VH), and a light chain variable domain sequence (VL). In some embodiments, an anti-HHLA2 antibody or antigen-binding fragment thereof comprises an immunoglobulin molecule of four polypeptide chains, e.g., two heavy chains and two light chains. A heavy chain can include a VH and a heavy chain constant domain. A heavy chain constant domain can include CH1, hinge, CH2, CH3, and optionally, a CH4 region. A light chain can include a VL and a light chain constant domain. A light chain constant domain can include a CL domain.

A VH and/or a VL can be further subdivided into regions of variability, termed complementarity determining regions (CDRs), interspersed with regions that are more conserved, termed framework regions (FR). Such VH and/or VL domains can each include three CDRs and four framework regions, arranged from amino-terminus to carboxyl-terminus in the following order: FR1, CDR1, FR2, CDR2, FR3, CDR3, FR4, one or more of which can be engineered as described herein. In general, there are three CDRs in each VH (HCDR1, HCDR2, and HCDR3) and three CDRs in each VL (LCDR1, LCDR2, and LCDR3). The extent of the framework region and CDRs can be defined using a number of well-known schemes (see, e.g., Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software, each of which is hereby incorporated by reference in its entirety).

An anti-HHLA2 antibody or antigen-binding fragment thereof described herein can be from any class of antibodies including, but not limited to, IgG, IgA, IgM, IgD, and IgE, and from any subclass (e.g., IgG1, IgG2, IgG3, and IgG4) of antibodies. An anti-HHLA2 antibody or antigen-binding fragment thereof described herein can be or comprise a human, humanized, CDR-grafted, or in vitro generated antibody. An anti-HHLA2 antibody or antigen-binding fragment thereof described herein can have or comprise a heavy chain constant region chosen from, e.g., IgG1, IgG2, IgG3, or IgG4. An anti-HHLA2 antibody or fragment can have or comprise a light chain chosen from, e.g., kappa or lambda.

In some embodiments, an anti-HHLA2 antibody or antigen-binding fragment thereof described herein is or comprises a monoclonal antibody. Typically, monoclonal antibodies are obtained from a population of substantially homogeneous antibodies, such that the individual antibodies comprising the population are substantially identical, except for possible naturally occurring mutations that may be present in minor amounts. Thus, the modifier “monoclonal” as used herein, indicates the character of the antibody as not being a mixture of discrete antibodies. In some embodiments, monoclonal antibodies directed to a particular epitope are derived from a single cell line (e.g., a B cell line).

In some embodiments, an anti-HHLA2 antibody or antigen-binding fragment thereof described herein is or comprises a polyclonal antibody. In contrast to monoclonal antibodies, polyclonal antibodies are typically obtained from a population of heterogeneous antibodies, such that the antibodies in a particular population include structural variation, for example, affinity for different epitopes on a particular target (e.g., HHLA2). Several methods of producing polyclonal antibodies are known in the art, including use of multiple subcutaneous and/or intraperitoneal injections of the relevant antigen into an animal, optionally including co-administration of one or more adjuvants.

In some embodiments, an anti-HHLA2 antibody or an antigen-binding fragment thereof described herein comprises: (a) a VH comprising one, two, or three VH CDR sequences each with at least about 80%, 85%, 90%, 91%, 92%, 93%, 94%, 95%, 96%, 97%, 98%, 99%, 99.5% or more identity to a VH CDR in Table 1; and/or (b) a VL comprising one, two, or three VL CDR sequences each with at least about 80%, 85%, 90%, 91%, 92%, 93%, 94%, 95%, 96%, 97%, 98%, 99%, 99.5% or more identity to a VL CDR in Table 1. In some embodiments, an anti-HHLA2 antibody or an antigen-binding fragment thereof described herein comprises: (a) a VH with at least about 80%, 85%, 90%, 91%, 92%, 93%, 94%, 95%, 96%, 97%, 98%, 99%, 99.5% or more identity to a VH in Table 1; and/or (a) a VL with at least about 80%, 85%, 90%, 91%, 92%, 93%, 94%, 95%, 96%, 97%, 98%, 99%, 99.5% or more identity to a VL in Table 1. In some embodiments, an anti-HHLA2 antibody or an antigen-binding fragment thereof described herein comprises: (a) a heavy chain with at least about 80%, 85%, 90%, 91%, 92%, 93%, 94%, 95%, 96%, 97%, 98%, 99%, 99.5% or more identity to a heavy chain in Table 1; and/or (a) a light chain with at least about 80%, 85%, 90%, 91%, 92%, 93%, 94%, 95%, 96%, 97%, 98%, 99%, 99.5% or more identity to a light chain in Table 1.

In some embodiments, an anti-HHLA2 antibody or an antigen-binding fragment thereof described herein comprises: (a) a VH comprising a VH CDR1 amino acid sequence of SEQ ID NO: 1, a VH CDR2 amino acid sequence of SEQ ID NO: 2, and a VH CDR3 amino acid sequence of SEQ ID NO: 3; and (b) a VL comprising a VL CDR1 amino acid sequence of SEQ ID NO: 14, a VL CDR2 amino acid sequence of SEQ ID NO: 15, and a VL CDR3 amino acid sequence of SEQ ID NO: 16. In some embodiments, an anti-HHLA2 antibody or an antigen-binding fragment thereof described herein comprises: (a) a VH comprising a VH CDR1 amino acid sequence of SEQ ID NO: 4, a VH CDR2 amino acid sequence of SEQ ID NO: 5, and a VH CDR3 amino acid sequence of SEQ ID NO: 6; and (b) a VL comprising a VL CDR1 amino acid sequence of SEQ ID NO: 17, a VL CDR2 amino acid sequence of SEQ ID NO: 18, and a VL CDR3 amino acid sequence of SEQ ID NO: 19, each disclosed in Table 1. In some embodiments, an anti-HHLA2 antibody or an antigen-binding fragment thereof described herein comprises: (a) a VH comprising a VH CDR1 amino acid sequence of SEQ ID NO: 7, a VH CDR2 amino acid sequence of SEQ ID NO: 8, and a VH CDR3 amino acid sequence of SEQ ID NO: 9; and (b) a VL comprising a VL CDR1 amino acid sequence of SEQ ID NO: 20, a VL CDR2 amino acid sequence of SEQ ID NO: 21, and a VL CDR3 amino acid sequence of SEQ ID NO: 22, each disclosed in Table 1.

In some embodiments, an anti-HHLA2 antibody or an antigen-binding fragment thereof described herein comprises a VH comprising an amino acid sequence of SEQ ID NO: 10, or an amino acid sequence at least 85%, 90%, 95%, or 99% identical or higher to SEQ ID NO: 10. In some embodiments, an anti-HHLA2 antibody or an antigen-binding fragment thereof described herein comprises a VL comprising an amino acid sequence of SEQ ID NO: 23, or an amino acid sequence at least 85%, 90%, 95%, or 99% identical or higher to SEQ ID NO: 23. In some embodiments, an anti-HHLA2 antibody or an antigen-binding fragment thereof described herein comprises a VH comprising an amino acid sequence of SEQ ID NO: 10 and a VL comprising an amino acid sequence of SEQ ID NO: 23.

In some embodiments, an anti-HHLA2 antibody or an antigen-binding fragment thereof described herein comprises a heavy chain comprising an amino acid sequence of SEQ ID NO: 12, or an amino acid sequence at least 85%, 90%, 95%, or 99% identical or higher to SEQ ID NO: 12. In some embodiments, an anti-HHLA2 antibody or an antigen-binding fragment thereof described herein comprises a light chain comprising an amino acid sequence of SEQ ID NO: 25, or an amino acid sequence at least 85%, 90%, 95%, or 99% identical or higher to SEQ ID NO: 25. In some embodiments, an anti-HHLA2 antibody or an antigen-binding fragment thereof described herein comprises a heavy chain comprising an amino acid sequence of SEQ ID NO: 12 and a light chain comprising an amino acid sequence of SEQ ID NO: 25.

In some embodiments, an anti-HHLA2 antibody or an antigen-binding fragment thereof described herein comprises: (a) a VH comprising a VH CDR1 amino acid sequence of SEQ ID NO: 27, a VH CDR2 amino acid sequence of SEQ ID NO: 28, and a VH CDR3 amino acid sequence of SEQ ID NO: 29; and (b) a VL comprising a VL CDR1 amino acid sequence of SEQ ID NO: 40, a VL CDR2 amino acid sequence of SEQ ID NO: 41, and a VL CDR3 amino acid sequence of SEQ ID NO: 42. In some embodiments, an anti-HHLA2 antibody or an antigen-binding fragment thereof described herein comprises: (a) a VH comprising a VH CDR1 amino acid sequence of SEQ ID NO: 30, a VH CDR2 amino acid sequence of SEQ ID NO: 31, and a VH CDR3 amino acid sequence of SEQ ID NO: 32; and (b) a VL comprising a VL CDR1 amino acid sequence of SEQ ID NO: 43, a VL CDR2 amino acid sequence of SEQ ID NO: 44, and a VL CDR3 amino acid sequence of SEQ ID NO: 45. In some embodiments, an anti-HHLA2 antibody or an antigen-binding fragment thereof described herein comprises: (a) a VH comprising a VH CDR1 amino acid sequence of SEQ ID NO: 33, a VH CDR2 amino acid sequence of SEQ ID NO: 34, and a VH CDR3 amino acid sequence of SEQ ID NO: 35; and (b) a VL comprising a VL CDR1 amino acid sequence of SEQ ID NO: 46, a VL CDR2 amino acid sequence of SEQ ID NO: 47, and a VL CDR3 amino acid sequence of SEQ ID NO: 48.

In some embodiments, an anti-HHLA2 antibody or an antigen-binding fragment thereof described herein comprises a VH comprising an amino acid sequence of SEQ ID NO: 36, or an amino acid sequence at least 85%, 90%, 95%, or 99% identical or higher to SEQ ID NO: 36. In some embodiments, an anti-HHLA2 antibody or an antigen-binding fragment thereof described herein comprises a VL comprising an amino acid sequence of SEQ ID NO: 49, or an amino acid sequence at least 85%, 90%, 95%, or 99% identical or higher to SEQ ID NO: 49. In some embodiments, an anti-HHLA2 antibody or an antigen-binding fragment thereof described herein comprises a VH comprising an amino acid sequence of SEQ ID NO: 36 and a VL comprising an amino acid sequence of SEQ ID NO: 49.

In some embodiments, an anti-HHLA2 antibody or an antigen-binding fragment thereof described herein comprises a heavy chain comprising an amino acid sequence of SEQ ID NO: 38, or an amino acid sequence at least 85%, 90%, 95%, or 99% identical or higher to SEQ ID NO: 38. In some embodiments, an anti-HHLA2 antibody or an antigen-binding fragment thereof described herein comprises a light chain comprising an amino acid sequence of SEQ ID NO: 51, or an amino acid sequence at least 85%, 90%, 95%, or 99% identical or higher to SEQ ID NO: 51. In some embodiments, an anti-HHLA2 antibody or an antigen-binding fragment thereof described herein comprises a heavy chain comprising an amino acid sequence of SEQ ID NO: 38, and a light chain comprising an amino acid sequence of SEQ ID NO: 51.

In some embodiments, an anti-HHLA2 antibody or an antigen-binding fragment thereof described herein comprises: (a) a VH comprising a VH CDR1 amino acid sequence of SEQ ID NO: 53, a VH CDR2 amino acid sequence of SEQ ID NO: 54, and a VH CDR3 amino acid sequence of SEQ ID NO: 55; and (b) a VL comprising a VL CDR1 amino acid sequence of SEQ ID NO: 66, a VL CDR2 amino acid sequence of SEQ ID NO: 67, and a VL CDR3 amino acid sequence of SEQ ID NO: 68. In some embodiments, an anti-HHLA2 antibody or an antigen-binding fragment thereof described herein comprises: (a) a VH comprising a VH CDR1 amino acid sequence of SEQ ID NO: 56, a VH CDR2 amino acid sequence of SEQ ID NO: 57, and a VH CDR3 amino acid sequence of SEQ ID NO: 58; and (b) a VL comprising a VL CDR1 amino acid sequence of SEQ ID NO: 69, a VL CDR2 amino acid sequence of SEQ ID NO: 70, and a VL CDR3 amino acid sequence of SEQ ID NO: 71. In some embodiments, an anti-HHLA2 antibody or an antigen-binding fragment thereof described herein comprises: (a) a VH comprising a VH CDR1 amino acid sequence of SEQ ID NO: 59, a VH CDR2 amino acid sequence of SEQ ID NO: 60, and a VH CDR3 amino acid sequence of SEQ ID NO: 61; and (b) a VL comprising a VL CDR1 amino acid sequence of SEQ ID NO: 72, a VL CDR2 amino acid sequence of SEQ ID NO: 73, and a VL CDR3 amino acid sequence of SEQ ID NO: 74.

In some embodiments, an anti-HHLA2 antibody or an antigen-binding fragment thereof described herein comprises a VH comprising an amino acid sequence of SEQ ID NO: 62, or an amino acid sequence at least 85%, 90%, 95%, or 99% identical or higher to SEQ ID NO: 62. In some embodiments, an anti-HHLA2 antibody or an antigen-binding fragment thereof described herein comprises a VL comprising an amino acid sequence of SEQ ID NO: 75, or an amino acid sequence at least 85%, 90%, 95%, or 99% identical or higher to SEQ ID NO: 75. In some embodiments, an anti-HHLA2 antibody or an antigen-binding fragment thereof described herein comprises a VH comprising an amino acid sequence of SEQ ID NO: 62 and a VL comprising an amino acid sequence of SEQ ID NO: 75.

In some embodiments, an anti-HHLA2 antibody or an antigen-binding fragment thereof described herein comprises a heavy chain comprising an amino acid sequence of SEQ ID NO: 64, or an amino acid sequence at least 85%, 90%, 95%, or 99% identical or higher to SEQ ID NO: 64. In some embodiments, an anti-HHLA2 antibody or an antigen-binding fragment thereof described herein comprises a light chain comprising an amino acid sequence of SEQ ID NO: 77, or an amino acid sequence at least 85%, 90%, 95%, or 99% identical or higher to SEQ ID NO: 77. In some embodiments, an anti-HHLA2 antibody or an antigen-binding fragment thereof described herein comprises a heavy chain comprising an amino acid sequence of SEQ ID NO: 64, and a light chain comprising an amino acid sequence of SEQ ID NO: 77.

In some embodiments, an anti-HHLA2 antibody or an antigen-binding fragment thereof described herein comprises: (a) a VH comprising a VH CDR1 amino acid sequence of SEQ ID NO: 79, a VH CDR2 amino acid sequence of SEQ ID NO: 80, and a VH CDR3 amino acid sequence of SEQ ID NO: 81; and (b) a VL comprising a VL CDR1 amino acid sequence of SEQ ID NO: 92, a VL CDR2 amino acid sequence of SEQ ID NO: 93, and a VL CDR3 amino acid sequence of SEQ ID NO: 94. In some embodiments, an anti-HHLA2 antibody or an antigen-binding fragment thereof described herein comprises: (a) a VH comprising a VH CDR1 amino acid sequence of SEQ ID NO: 82, a VH CDR2 amino acid sequence of SEQ ID NO: 83, and a VH CDR3 amino acid sequence of SEQ ID NO: 84; and (b) a VL comprising a VL CDR1 amino acid sequence of SEQ ID NO: 95, a VL CDR2 amino acid sequence of SEQ ID NO: 96, and a VL CDR3 amino acid sequence of SEQ ID NO: 97. In some embodiments, an anti-HHLA2 antibody or an antigen-binding fragment thereof described herein comprises: (a) a VH comprising a VH CDR1 amino acid sequence of SEQ ID NO: 85, a VH CDR2 amino acid sequence of SEQ ID NO: 86, and a VH CDR3 amino acid sequence of SEQ ID NO: 87; and (b) a VL comprising a VL CDR1 amino acid sequence of SEQ ID NO: 98, a VL CDR2 amino acid sequence of SEQ ID NO: 99, and a VL CDR3 amino acid sequence of SEQ ID NO: 100.

In some embodiments, an anti-HHLA2 antibody or an antigen-binding fragment thereof described herein comprises a VH comprising an amino acid sequence of SEQ ID NO: 88, or an amino acid sequence at least 85%, 90%, 95%, or 99% identical or higher to SEQ ID NO: 88. In some embodiments, an anti-HHLA2 antibody or an antigen-binding fragment thereof described herein comprises a VL comprising an amino acid sequence of SEQ ID NO: 101, or an amino acid sequence at least 85%, 90%, 95%, or 99% identical or higher to SEQ ID NO: 101. In some embodiments, an anti-HHLA2 antibody or an antigen-binding fragment thereof described herein comprises a VH comprising an amino acid sequence of SEQ ID NO: 88 and a VL comprising an amino acid sequence of SEQ ID NO: 101.

In some embodiments, an anti-HHLA2 antibody or an antigen-binding fragment thereof described herein comprises a heavy chain comprising an amino acid sequence of SEQ ID NO: 90, or an amino acid sequence at least 85%, 90%, 95%, or 99% identical or higher to SEQ ID NO: 90. In some embodiments, an anti-HHLA2 antibody or an antigen-binding fragment thereof described herein comprises a light chain comprising an amino acid sequence of SEQ ID NO: 103, or an amino acid sequence at least 85%, 90%, 95%, or 99% identical or higher to SEQ ID NO: 103. In some embodiments, an anti-HHLA2 antibody or an antigen-binding fragment thereof described herein comprises a heavy chain comprising an amino acid sequence of SEQ ID NO: 90, and a light chain comprising an amino acid sequence of SEQ ID NO: 103.

In some embodiments, an anti-HHLA2 antibody or an antigen-binding fragment thereof described herein comprises: (a) a VH comprising a VH CDR1 amino acid sequence of SEQ ID NO: 105, a VH CDR2 amino acid sequence of SEQ ID NO: 106, and a VH CDR3 amino acid sequence of SEQ ID NO: 107; and (b) a VL comprising a VL CDR1 amino acid sequence of SEQ ID NO: 118, a VL CDR2 amino acid sequence of SEQ ID NO: 119, and a VL CDR3 amino acid sequence of SEQ ID NO: 120. In some embodiments, an anti-HHLA2 antibody or an antigen-binding fragment thereof described herein comprises: (a) a VH comprising a VH CDR1 amino acid sequence of SEQ ID NO: 108, a VH CDR2 amino acid sequence of SEQ ID NO: 109, and a VH CDR3 amino acid sequence of SEQ ID NO: 110; and (b) a VL comprising a VL CDR1 amino acid sequence of SEQ ID NO: 121, a VL CDR2 amino acid sequence of SEQ ID NO: 122, and a VL CDR3 amino acid sequence of SEQ ID NO: 123. In some embodiments, an anti-HHLA2 antibody or an antigen-binding fragment thereof described herein comprises: (a) a VH comprising a VH CDR1 amino acid sequence of SEQ ID NO: 111, a VH CDR2 amino acid sequence of SEQ ID NO: 112, and a VH CDR3 amino acid sequence of SEQ ID NO: 113; and (b) a VL comprising a VL CDR1 amino acid sequence of SEQ ID NO: 124, a VL CDR2 amino acid sequence of SEQ ID NO: 125, and a VL CDR3 amino acid sequence of SEQ ID NO: 126.

In some embodiments, an anti-HHLA2 antibody or an antigen-binding fragment thereof described herein comprises a VH comprising an amino acid sequence of SEQ ID NO: 114, or an amino acid sequence at least 85%, 90%, 95%, or 99% identical or higher to SEQ ID NO: 114. In some embodiments, an anti-HHLA2 antibody or an antigen-binding fragment thereof described herein comprises a VL comprising an amino acid sequence of SEQ ID NO: 127, or an amino acid sequence at least 85%, 90%, 95%, or 99% identical or higher to SEQ ID NO: 127. In some embodiments, an anti-HHLA2 antibody or an antigen-binding fragment thereof described herein comprises a VH comprising an amino acid sequence of SEQ ID NO: 114 and a VL comprising an amino acid sequence of SEQ ID NO: 127.

In some embodiments, an anti-HHLA2 antibody or an antigen-binding fragment thereof described herein comprises a heavy chain comprising an amino acid sequence of SEQ ID NO: 116, or an amino acid sequence at least 85%, 90%, 95%, or 99% identical or higher to SEQ ID NO: 116. In some embodiments, an anti-HHLA2 antibody or an antigen-binding fragment thereof described herein comprises a light chain comprising an amino acid sequence of SEQ ID NO: 129, or an amino acid sequence at least 85%, 90%, 95%, or 99% identical or higher to SEQ ID NO: 129. In some embodiments, an anti-HHLA2 antibody or an antigen-binding fragment thereof described herein comprises a heavy chain comprising an amino acid sequence of SEQ ID NO: 116, and a light chain comprising an amino acid sequence of SEQ ID NO: 129.

In some embodiments, an anti-HHLA2 antibody or an antigen-binding fragment thereof described herein comprises: (a) a VH comprising a VH CDR1 amino acid sequence of SEQ ID NO: 131, a VH CDR2 amino acid sequence of SEQ ID NO: 132, and a VH CDR3 amino acid sequence of SEQ ID NO: 133; and (b) a VL comprising a VL CDR1 amino acid sequence of SEQ ID NO: 144, a VL CDR2 amino acid sequence of SEQ ID NO: 145, and a VL CDR3 amino acid sequence of SEQ ID NO: 146. In some embodiments, an anti-HHLA2 antibody or an antigen-binding fragment thereof described herein comprises: (a) a VH comprising a VH CDR1 amino acid sequence of SEQ ID NO: 134, a VH CDR2 amino acid sequence of SEQ ID NO: 135, and a VH CDR3 amino acid sequence of SEQ ID NO: 136; and (b) a VL comprising a VL CDR1 amino acid sequence of SEQ ID NO: 147, a VL CDR2 amino acid sequence of SEQ ID NO: 148, and a VL CDR3 amino acid sequence of SEQ ID NO: 149. In some embodiments, an anti-HHLA2 antibody or an antigen-binding fragment thereof described herein comprises: (a) a VH comprising a VH CDR1 amino acid sequence of SEQ ID NO: 137, a VH CDR2 amino acid sequence of SEQ ID NO: 138, and a VH CDR3 amino acid sequence of SEQ ID NO: 139; and (b) a VL comprising a VL CDR1 amino acid sequence of SEQ ID NO: 150, a VL CDR2 amino acid sequence of SEQ ID NO: 151, and a VL CDR3 amino acid sequence of SEQ ID NO: 152.

In some embodiments, an anti-HHLA2 antibody or an antigen-binding fragment thereof described herein comprises a VH comprising an amino acid sequence of SEQ ID NO: 140, or an amino acid sequence at least 85%, 90%, 95%, or 99% identical or higher to SEQ ID NO: 140. In some embodiments, an anti-HHLA2 antibody or an antigen-binding fragment thereof described herein comprises a VL comprising an amino acid sequence of SEQ ID NO: 153, or an amino acid sequence at least 85%, 90%, 95%, or 99% identical or higher to SEQ ID NO: 153. In some embodiments, an anti-HHLA2 antibody or an antigen-binding fragment thereof described herein comprises a VH comprising an amino acid sequence of SEQ ID NO: 140 and a VL comprising an amino acid sequence of SEQ ID NO: 153.

In some embodiments, an anti-HHLA2 antibody or an antigen-binding fragment thereof described herein comprises a heavy chain comprising an amino acid sequence of SEQ ID NO: 142, or an amino acid sequence at least 85%, 90%, 95%, or 99% identical or higher to SEQ ID NO: 142. In some embodiments, an anti-HHLA2 antibody or an antigen-binding fragment thereof described herein comprises a light chain comprising an amino acid sequence of SEQ ID NO: 155, or an amino acid sequence at least 85%, 90%, 95%, or 99% identical or higher to SEQ ID NO: 155. In some embodiments, an anti-HHLA2 antibody or an antigen-binding fragment thereof described herein comprises a heavy chain comprising an amino acid sequence of SEQ ID NO: 142, and a light chain comprising an amino acid sequence of SEQ ID NO: 155.

In some embodiments, an anti-HHLA2 antibody or an antigen-binding fragment thereof described herein comprises: (a) a VH comprising a VH CDR1 amino acid sequence of SEQ ID NO: 157, a VH CDR2 amino acid sequence of SEQ ID NO: 158, and a VH CDR3 amino acid sequence of SEQ ID NO: 159; and (b) a VL comprising a VL CDR1 amino acid sequence of SEQ ID NO: 170, a VL CDR2 amino acid sequence of SEQ ID NO: 171, and a VL CDR3 amino acid sequence of SEQ ID NO: 172. In some embodiments, an anti-HHLA2 antibody or an antigen-binding fragment thereof described herein comprises: (a) a VH comprising a VH CDR1 amino acid sequence of SEQ ID NO: 160, a VH CDR2 amino acid sequence of SEQ ID NO: 161, and a VH CDR3 amino acid sequence of SEQ ID NO: 162; and (b) a VL comprising a VL CDR1 amino acid sequence of SEQ ID NO: 173, a VL CDR2 amino acid sequence of SEQ ID NO: 174, and a VL CDR3 amino acid sequence of SEQ ID NO: 175. In some embodiments, an anti-HHLA2 antibody or an antigen-binding fragment thereof described herein comprises: (a) a VH comprising a VH CDR1 amino acid sequence of SEQ ID NO: 163, a VH CDR2 amino acid sequence of SEQ ID NO: 164, and a VH CDR3 amino acid sequence of SEQ ID NO: 165; and (b) a VL comprising a VL CDR1 amino acid sequence of SEQ ID NO: 176, a VL CDR2 amino acid sequence of SEQ ID NO: 177, and a VL CDR3 amino acid sequence of SEQ ID NO: 178.

In some embodiments, an anti-HHLA2 antibody or an antigen-binding fragment thereof described herein comprises a VH comprising an amino acid sequence of SEQ ID NO: 166, or an amino acid sequence at least 85%, 90%, 95%, or 99% identical or higher to SEQ ID NO: 166. In some embodiments, an anti-HHLA2 antibody or an antigen-binding fragment thereof described herein comprises a VL comprising an amino acid sequence of SEQ ID NO: 179, or an amino acid sequence at least 85%, 90%, 95%, or 99% identical or higher to SEQ ID NO: 179. In some embodiments, an anti-HHLA2 antibody or an antigen-binding fragment thereof described herein comprises a VH comprising an amino acid sequence of SEQ ID NO: 166 and a VL comprising an amino acid sequence of SEQ ID NO: 179.

In some embodiments, an anti-HHLA2 antibody or an antigen-binding fragment thereof described herein comprises a heavy chain comprising an amino acid sequence of SEQ ID NO: 168, or an amino acid sequence at least 85%, 90%, 95%, or 99% identical or higher to SEQ ID NO: 168. In some embodiments, an anti-HHLA2 antibody or an antigen-binding fragment thereof described herein comprises a light chain comprising an amino acid sequence of SEQ ID NO: 181, or an amino acid sequence at least 85%, 90%, 95%, or 99% identical or higher to SEQ ID NO: 181. In some embodiments, an anti-HHLA2 antibody or an antigen-binding fragment thereof described herein comprises a heavy chain comprising an amino acid sequence of SEQ ID NO: 168, and a light chain comprising an amino acid sequence of SEQ ID NO: 181.

TABLE 1
Amino acid and nucleotide sequences of exemplary anti-HHLA2 antibodies.

| | | SEQ ID |
|---|---|---|
| Clone | Sequences | NO |
| Ab- | Heavy Chain | |
| 60638 | HCDR1 (IMGT) | SEQ ID |
| | GYTFTSYA | NO: 1 |
| | HCDR2 (IMGT) | SEQ ID |
| | INAGTGNT | NO: 2 |
| | HCDR3 (IMGT) | SEQ ID |
| | ARDLPHRGAFDP | NO: 3 |
| | HCDR1 (Kabat) | SEQ ID |
| | SYAMH | NO: 4 |
| | HCDR2 (Kabat) | SEQ ID |
| | WINAGTGNTKYSQKFQG | NO: 5 |
| | HCDR3 (Kabat) | SEQ ID |
| | DLPHRGAFDP | NO: 6 |
| | HCDR1 (Combined Kabat/IMGT) | SEQ ID |
| | GYTFTSYAMH | NO: 7 |
| | HCDR2 (Combined Kabat/IMGT) | SEQ ID |
| | WINAGTGNTKYSQKFQG | NO: 8 |
| | HCDR3 (Combined Kabat/IMGT) | SEQ ID |
| | DLPHRGAFDP | NO: 9 |
| | VH-Amino acid sequence | SEQ ID |
| | QVQLVQSGAEVKKPGASVKVSCKASGYTFTSYAMHWVRQAP | NO: 10 |
| | GQRLEWMGWINAGTGNTKYSQKFQGRVTITRDTSASTAYMEL | |
| | SSLRSEDTAVYYCARDLPHRGAFDPWGQGTLVTVSS | |
| | VH-DNA sequence | SEQ ID |
| | CAGGTGCAGCTGGTGCAGTCTGGGGCTGAGGTGAAGAAGCC | NO: 11 |
| | TGGGGCCTCAGTGAAGGTTTCCTGCAAGGCTTCTGGATACAC | |
| | CTTCACTAGCTATGCTATGCATTGGGTGCGCCAGGCCCCCGG | |
| | ACAAAGGCTTGAGTGGATGGGATGGATCAACGCTGGCACTG | |
| | GTAACACAAAATATTCACAAAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGATCTTCCTCACAGAGGAGCATTCGACCCATGGGGA | |
| | CAGGGTACATTGGTCACCGTCTCCTCA | |
| | Heavy chain-Amino acid sequence | SEQ ID |
| | QVQLVQSGAEVKKPGASVKVSCKASGYTFTSYAMHWVRQAP | NO: 12 |
| | GQRLEWMGWINAGTGNTKYSQKFQGRVTITRDTSASTAYMEL | |
| | SSLRSEDTAVYYCARDLPHRGAFDPWGQGTLVTVSSASTKGPS | |
| | VFPLAPCSRSTSESTAALGCLVKDYFPEPVTVSWNSGALTSGVH | |
| | TFPAVLQSSGLYSLSSVVTVPSSSLGTKTYTCNVDHKPSNTKVD | |
| | KRVESKYGPPCPPCPAPEFLGGPSVFLFPPKPKDTLMISRTPEVT | |
| | CVVVDVSQEDPEVQFNWYVDGVEVHNAKTKPREEQFNSTYRV | |
| | VSVLTVLHQDWLNGKEYKCKVSNKGLPSSIEKTISKAKGQPRE | |
| | PQVYTLPPSQEEMTKNQVSLTCLVKGFYPSDIAVEWESNGQPE | |
| | NNYKTTPPVLDSDGSFFLYSRLTVDKSRWQEGNVFSCSVMHEA | |
| | LHNHYTQKSLSLSLGK | |
| | Heavy chain-DNA sequence | SEQ ID |
| | CAGGTGCAGCTGGTGCAGTCTGGGGCTGAGGTGAAGAAGCC | NO: 13 |
| | TGGGGCCTCAGTGAAGGTTTCCTGCAAGGCTTCTGGATACAC | |
| | CTTCACTAGCTATGCTATGCATTGGGTGCGCCAGGCCCCCGG | |
| | ACAAAGGCTTGAGTGGATGGGATGGATCAACGCTGGCACTG | |
| | GTAACACAAAATATTCACAAAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGATCTTCCTCACAGAGGAGCATTCGACCCATGGGGA | |
| | CAGGGTACATTGGTCACCGTCTCCTCAGCTAGCACCAAGGGC | |
| | CCATCCGTCTTCCCCCTGGCGCCCTGCTCCAGGAGCACCTCC | |
| | GAGAGCACAGCCGCCCTGGGCTGCCTGGTCAAGGACTACTT | |
| | CCCCGAACCGGTGACGGTGTCGTGGAACTCAGGCGCCCTGA | |
| | CCAGCGGCGTGCACACCTTCCCGGCTGTCCTACAGTCCTCAG | |
| | GACTCTACTCCCTCAGCAGCGTGGTGACCGTGCCCTCCAGCA | |
| | GCTTGGGCACGAAGACCTACACCTGCAACGTAGATCACAAG | |
| | CCCAGCAACACCAAGGTGGACAAGAGAGTTGAGTCCAAATA | |
| | TGGTCCCCCATGCCCACCATGCCCAGCACCTGAGTTCCTGGG | |
| | GGGACCATCAGTCTTCCTGTTCCCCCCAAAACCCAAGGACAC | |
| | TCTCATGATCTCCCGGACCCCTGAGGTCACGTGCGTGGTGGT | |
| | GGACGTGAGCCAGGAAGACCCCGAGGTCCAGTTCAACTGGT | |
| | ACGTGGATGGCGTGGAGGTGCATAATGCCAAGACAAAGCCG | |
| | CGGGAGGAGCAGTTCAACAGCACGTACCGTGTGGTCAGCGT | |
| | CCTCACCGTCCTGCACCAGGACTGGCTGAACGGCAAGGAGT | |
| | ACAAGTGCAAGGTCTCCAACAAAGGCCTCCCGTCCTCCATCG | |
| | AGAAAACCATCTCCAAAGCCAAAGGGCAGCCCCGAGAGCCA | |
| | CAGGTGTACACCCTGCCCCCATCCCAGGAGGAGATGACCAA | |
| | GAACCAGGTCAGCCTGACCTGCCTGGTCAAAGGCTTCTACCC | |
| | CAGCGACATCGCCGTGGAGTGGGAGAGCAATGGGCAGCCGG | |
| | AGAACAACTACAAGACCACGCCTCCCGTGCTGGACTCCGAC | |
| | GGCTCCTTCTTCCTCTACAGCAGGCTAACCGTGGACAAGAGC | |
| | AGGTGGCAGGAGGGGAATGTCTTCTCATGCTCCGTGATGCAT | |
| | GAGGCTCTGCACAACCACTACACACAGAAGAGCCTCTCCCT | |
| | GTCTCTGGGCAAA | |
| | Light Chain | |
| | LCDR1 (IMGT) | SEQ ID |
| | QSVSSDY | NO: 14 |
| | LCDR2 (IMGT) | SEQ ID |
| | GAS | NO: 15 |
| | LCDR3 (IMGT) | SEQ ID |
| | QLYGSAPRT | NO: 16 |
| | LCDR1 (Kabat) | SEQ ID |
| | RASQSVSSDYLA | NO: 17 |
| | LCDR2 (Kabat) | SEQ ID |
| | GASSRAT | NO: 18 |
| | LCDR3 (Kabat) | SEQ ID |
| | QLYGSAPRT | NO: 19 |
| | LCDR1 (Combined Kabat/IMGT) | SEQ ID |
| | RASQSVSSDYLA | NO: 20 |
| | LCDR2 (Combined Kabat/IMGT) | SEQ ID |
| | GASSRAT | NO: 21 |
| | LCDR3 (Combined Kabat/IMGT) | SEQ ID |
| | QLYGSAPRT | NO: 22 |
| | VL-Amino acid sequence | SEQ ID |
| | EIVLTQSPGTLSLSPGERATLSCRASQSVSSDYLAWYQQKPGQA | NO: 23 |
| | PRLLIYGASSRATGIPDRFSGSGSGTDFTLTISRLEPEDFAVYYCQ | |
| | LYGSAPRTFGGGTKVEIK | |
| | VL-DNA sequence | SEQ ID |
| | GAAATTGTGTTGACGCAGTCTCCAGGCACCCTGTCTTTGTCT | NO: 24 |
| | CCAGGGGAAAGAGCCACCCTCTCCTGCAGGGCCAGTCAGAG | |
| | TGTTAGCAGCGACTACTTAGCCTGGTACCAGCAGAAACCTGG | |
| | CCAGGCTCCCAGGCTCCTCATCTATGGTGCATCCAGCAGGGC | |
| | CACTGGCATCCCAGACAGGTTCAGTGGCAGTGGGTCTGGGA | |
| | CAGACTTCACTCTCACCATCAGCAGACTGGAGCCTGAAGATT | |
| | TTGCAGTGTATTACTGTCAGCTATACGGAAGTGCCCCTAGGA | |
| | CTTTTGGCGGAGGGACCAAGGTTGAGATCAAA | |
| | Light chain-Amino acid sequence | SEQ ID |
| | EIVLTQSPGTLSLSPGERATLSCRASQSVSSDYLAWYQQKPGQA | NO: 25 |
| | PRLLIYGASSRATGIPDRFSGSGSGTDFTLTISRLEPEDFAVYYCQ | |
| | LYGSAPRTFGGGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | Light chain-DNA sequence | SEQ ID |
| | GAAATTGTGTTGACGCAGTCTCCAGGCACCCTGTCTTTGTCT | NO: 26 |
| | CCAGGGGAAAGAGCCACCCTCTCCTGCAGGGCCAGTCAGAG | |
| | TGTTAGCAGCGACTACTTAGCCTGGTACCAGCAGAAACCTGG | |
| | CCAGGCTCCCAGGCTCCTCATCTATGGTGCATCCAGCAGGGC | |
| | CACTGGCATCCCAGACAGGTTCAGTGGCAGTGGGTCTGGGA | |
| | CAGACTTCACTCTCACCATCAGCAGACTGGAGCCTGAAGATT | |
| | TTGCAGTGTATTACTGTCAGCTATACGGAAGTGCCCCTAGGA | |
| | CTTTTGGCGGAGGGACCAAGGTTGAGATCAAACGTACGGTG | |
| | GCTGCACCATCTGTCTTCATCTTCCCGCCATCTGATGAGCAG | |
| | TTGAAATCTGGAACTGCCTCTGTTGTGTGCCTGCTGAATAAC | |
| | TTCTATCCCAGAGAGGCCAAAGTACAGTGGAAGGTGGATAA | |
| | CGCCCTCCAATCGGGTAACTCCCAGGAGAGTGTCACAGAGC | |
| | AGGACAGCAAGGACAGCACCTACAGCCTCAGCAGCACCCTG | |
| | ACGCTGAGCAAAGCAGACTACGAGAAACACAAAGTCTACGC | |
| | CTGCGAAGTCACCCATCAGGGCCTGAGCTCGCCCGTCACAA | |
| | AGAGCTTCAACAGGGGAGAGTGT | |
| Ab- | Heavy Chain | |
| 60665 | HCDR1 (IMGT) | SEQ ID |
| | GGTSSNYA | NO: 27 |
| | HCDR2 (IMGT) | SEQ ID |
| | IIPIFGTA | NO: 28 |
| | HCDR3 (IMGT) | SEQ ID |
| | ARSGTIWHDI | NO: 29 |
| | HCDR1 (Kabat) | SEQ ID |
| | NYAIS | NO: 30 |
| | HCDR2 (Kabat) | SEQ ID |
| | GIIPIFGTANYAQKFQG | NO: 31 |
| | HCDR3 (Kabat) | SEQ ID |
| | SGTIWHDI | NO: 32 |
| | HCDR1 (Combined Kabat/IMGT) | SEQ ID |
| | GGTSSNYAIS | NO: 33 |
| | HCDR2 (Combined Kabat/IMGT) | SEQ ID |
| | GIIPIFGTANYAQKFQG | NO: 34 |
| | HCDR3 (Combined Kabat/IMGT) | SEQ ID |
| | SGTIWHDI | NO: 35 |
| | VH-Amino acid sequence | SEQ ID |
| | QVQLVQSGAEVKKPGSSVKVSCKASGGTSSNYAISWVRQAPG | NO: 36 |
| | QGLEWMGGIIPIFGTANYAQKFQGRVTITADESTSTAYMELSSL | |
| | RSEDTAVYYCARSGTIWHDIWGQGTMVTVSS | |
| | VH-DNA sequence | SEQ ID |
| | CAGGTGCAGCTGGTGCAGTCTGGGGCTGAGGTGAAGAAGCC | NO: 37 |
| | TGGGTCCTCGGTGAAGGTCTCCTGCAAGGCTTCTGGAGGCAC | |
| | CTCCAGCAACTATGCTATCAGCTGGGTGCGACAGGCCCCTGG | |
| | ACAAGGGCTTGAGTGGATGGGAGGGATCATCCCTATCTTTGG | |
| | TACAGCAAACTACGCACAGAAGTTCCAGGGCAGAGTCACGA | |
| | TTACCGCGGACGAATCCACGAGCACAGCCTACATGGAGCTG | |
| | AGCAGCCTGAGATCTGAGGACACGGCGGTGTACTACTGCGC | |
| | CAGATCAGGAACAATATGGCACGACATATGGGGTCAGGGTA | |
| | CAATGGTCACCGTCTCCTCA | |
| | Heavy chain-Amino acid sequence | SEQ ID |
| | QVQLVQSGAEVKKPGSSVKVSCKASGGTSSNYAISWVRQAPG | NO: 38 |
| | QGLEWMGGIIPIFGTANYAQKFQGRVTITADESTSTAYMELSSL | |
| | RSEDTAVYYCARSGTIWHDIWGQGTMVTVSSASTKGPSVFPLA | |
| | PCSRSTSESTAALGCLVKDYFPEPVTVSWNSGALTSGVHTFPAV | |
| | LQSSGLYSLSSVVTVPSSSLGTKTYTCNVDHKPSNTKVDKRVES | |
| | KYGPPCPPCPAPEFLGGPSVFLFPPKPKDTLMISRTPEVTCVVVD | |
| | VSQEDPEVQFNWYVDGVEVHNAKTKPREEQFNSTYRVVSVLT | |
| | VLHQDWLNGKEYKCKVSNKGLPSSIEKTISKAKGQPREPQVYT | |
| | LPPSQEEMTKNQVSLTCLVKGFYPSDIAVEWESNGQPENNYKT | |
| | TPPVLDSDGSFFLYSRLTVDKSRWQEGNVFSCSVMHEALHNHY | |
| | TQKSLSLSLGK | |
| | Heavy chain-DNA sequence | SEQ ID |
| | CAGGTGCAGCTGGTGCAGTCTGGGGCTGAGGTGAAGAAGCC | NO: 39 |
| | TGGGTCCTCGGTGAAGGTCTCCTGCAAGGCTTCTGGAGGCAC | |
| | CTCCAGCAACTATGCTATCAGCTGGGTGCGACAGGCCCCTGG | |
| | ACAAGGGCTTGAGTGGATGGGAGGGATCATCCCTATCTTTGG | |
| | TACAGCAAACTACGCACAGAAGTTCCAGGGCAGAGTCACGA | |
| | TTACCGCGGACGAATCCACGAGCACAGCCTACATGGAGCTG | |
| | AGCAGCCTGAGATCTGAGGACACGGCGGTGTACTACTGCGC | |
| | CAGATCAGGAACAATATGGCACGACATATGGGGTCAGGGTA | |
| | CAATGGTCACCGTCTCCTCAGCTAGCACCAAGGGCCCATCCG | |
| | TCTTCCCCCTGGCGCCCTGCTCCAGGAGCACCTCCGAGAGCA | |
| | CAGCCGCCCTGGGCTGCCTGGTCAAGGACTACTTCCCCGAAC | |
| | CGGTGACGGTGTCGTGGAACTCAGGCGCCCTGACCAGCGGC | |
| | GTGCACACCTTCCCGGCTGTCCTACAGTCCTCAGGACTCTAC | |
| | TCCCTCAGCAGCGTGGTGACCGTGCCCTCCAGCAGCTTGGGC | |
| | ACGAAGACCTACACCTGCAACGTAGATCACAAGCCCAGCAA | |
| | CACCAAGGTGGACAAGAGAGTTGAGTCCAAATATGGTCCCC | |
| | CATGCCCACCATGCCCAGCACCTGAGTTCCTGGGGGGACCAT | |
| | CAGTCTTCCTGTTCCCCCCAAAACCCAAGGACACTCTCATGA | |
| | TCTCCCGGACCCCTGAGGTCACGTGCGTGGTGGTGGACGTGA | |
| | GCCAGGAAGACCCCGAGGTCCAGTTCAACTGGTACGTGGAT | |
| | GGCGTGGAGGTGCATAATGCCAAGACAAAGCCGCGGGAGGA | |
| | GCAGTTCAACAGCACGTACCGTGTGGTCAGCGTCCTCACCGT | |
| | CCTGCACCAGGACTGGCTGAACGGCAAGGAGTACAAGTGCA | |
| | AGGTCTCCAACAAAGGCCTCCCGTCCTCCATCGAGAAAACC | |
| | ATCTCCAAAGCCAAAGGGCAGCCCCGAGAGCCACAGGTGTA | |
| | CACCCTGCCCCCATCCCAGGAGGAGATGACCAAGAACCAGG | |
| | TCAGCCTGACCTGCCTGGTCAAAGGCTTCTACCCCAGCGACA | |
| | TCGCCGTGGAGTGGGAGAGCAATGGGCAGCCGGAGAACAAC | |
| | TACAAGACCACGCCTCCCGTGCTGGACTCCGACGGCTCCTTC | |
| | TTCCTCTACAGCAGGCTAACCGTGGACAAGAGCAGGTGGCA | |
| | GGAGGGGAATGTCTTCTCATGCTCCGTGATGCATGAGGCTCT | |
| | GCACAACCACTACACACAGAAGAGCCTCTCCCTGTCTCTGGG | |
| | CAAA | |
| | Light Chain | |
| | LCDR1 (IMGT) | SEQ ID |
| | QSVSSY | NO: 40 |
| | LCDR2 (IMGT) | SEQ ID |
| | DSS | NO: 41 |
| | LCDR3 (IMGT) | SEQ ID |
| | QQVVHWPPT | NO: 42 |
| | LCDR1 (Kabat) | SEQ ID |
| | RASQSVSSYLA | NO: 43 |
| | LCDR2 (Kabat) | SEQ ID |
| | DSSNRAT | NO: 44 |
| | LCDR3 (Kabat) | SEQ ID |
| | QQVVHWPPT | NO: 45 |
| | LCDR1 (Combined Kabat/IMGT) | SEQ ID |
| | RASQSVSSYLA | NO: 46 |
| | LCDR2 (Combined Kabat/IMGT) | SEQ ID |
| | DSSNRAT | NO: 47 |
| | LCDR3 (Combined Kabat/IMGT) | SEQ ID |
| | QQVVHWPPT | NO: 48 |
| | VL-Amino acid sequence | SEQ ID |
| | EIVLTQSPATLSLSPGERATLSCRASQSVSSYLAWYQQKPGQAP | NO: 49 |
| | RLLIYDSSNRATGIPARFSGSGSGTDFTLTISSLEPEDFAVYYCQ | |
| | QVVHWPPTFGGGTKVEIK | |
| | VL-DNA sequence | SEQ ID |
| | GAAATTGTGTTGACACAGTCTCCAGCCACCCTGTCTTTGTCT | NO: 50 |
| | CCAGGGGAAAGAGCCACCCTCTCCTGCAGGGCCAGTCAGAG | |
| | TGTTAGCAGCTACTTAGCCTGGTACCAACAGAAACCTGGCCA | |
| | GGCTCCCAGGCTCCTCATCTATGATTCATCCAACAGGGCCAC | |
| | TGGCATCCCAGCCAGGTTCAGTGGCAGTGGGTCTGGGACAG | |
| | ACTTCACTCTCACCATCAGCAGCCTAGAGCCTGAAGATTTTG | |
| | CAGTTTATTACTGTCAGCAGGTCGTCCACTGGCCTCCCACTTT | |
| | TGGCGGAGGGACCAAGGTTGAGATCAAA | |
| | Light chain-Amino acid sequence | SEQ ID |
| | EIVLTQSPATLSLSPGERATLSCRASQSVSSYLAWYQQKPGQAP | NO: 51 |
| | RLLIYDSSNRATGIPARFSGSGSGTDFTLTISSLEPEDFAVYYCQ | |
| | QVVHWPPTFGGGTKVEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | Light chain-DNA sequence | SEQ ID |
| | GAAATTGTGTTGACACAGTCTCCAGCCACCCTGTCTTTGTCT | NO: 52 |
| | CCAGGGGAAAGAGCCACCCTCTCCTGCAGGGCCAGTCAGAG | |
| | TGTTAGCAGCTACTTAGCCTGGTACCAACAGAAACCTGGCCA | |
| | GGCTCCCAGGCTCCTCATCTATGATTCATCCAACAGGGCCAC | |
| | TGGCATCCCAGCCAGGTTCAGTGGCAGTGGGTCTGGGACAG | |
| | ACTTCACTCTCACCATCAGCAGCCTAGAGCCTGAAGATTTTG | |
| | CAGTTTATTACTGTCAGCAGGTCGTCCACTGGCCTCCCACTTT | |
| | TGGCGGAGGGACCAAGGTTGAGATCAAACGTACGGTGGCTG | |
| | CACCATCTGTCTTCATCTTCCCGCCATCTGATGAGCAGTTGA | |
| | AATCTGGAACTGCCTCTGTTGTGTGCCTGCTGAATAACTTCT | |
| | ATCCCAGAGAGGCCAAAGTACAGTGGAAGGTGGATAACGCC | |
| | CTCCAATCGGGTAACTCCCAGGAGAGTGTCACAGAGCAGGA | |
| | CAGCAAGGACAGCACCTACAGCCTCAGCAGCACCCTGACGC | |
| | TGAGCAAAGCAGACTACGAGAAACACAAAGTCTACGCCTGC | |
| | GAAGTCACCCATCAGGGCCTGAGCTCGCCCGTCACAAAGAG | |
| | CTTCAACAGGGGAGAGTGT | |
| Ab- | Heavy Chain | |
| 65885 | HCDR1 (IMGT) | SEQ ID |
| | GYTFDSYK | NO: 53 |
| | HCDR2 (IMGT) | SEQ ID |
| | IPAGTGNT | NO: 54 |
| | HCDR3 (IMGT) | SEQ ID |
| | ARDRGGYYYDSWDD | NO: 55 |
| | HCDR1 (Kabat) | SEQ ID |
| | SYKMH | NO: 56 |
| | HCDR2 (Kabat) | SEQ ID |
| | YIPAGTGNTKYSQKFQG | NO: 57 |
| | HCDR3 (Kabat) | SEQ ID |
| | DRGGYYYDSWDD | NO: 58 |
| | HCDR1 (Combined Kabat/IMGT) | SEQ ID |
| | SGYTFDSYKMH | NO: 59 |
| | HCDR2 (Combined Kabat/IMGT) | SEQ ID |
| | YIPAGTGNTKYSQKFQG | NO: 60 |
| | HCDR3 (Combined Kabat/IMGT) | SEQ ID |
| | DRGGYYYDSWDD | NO: 61 |
| | VH-Amino acid sequence | SEQ ID |
| | QVQLVQSGAEVKKPGASVKVSCKASGYTFDSYKMHWVRQAP | NO: 62 |
| | GQRLEWMGYIPAGTGNTKYSQKFQGRVTITRDTSASTAYMELS | |
| | SLRSEDTAVYYCARDRGGYYYDSWDDWGQGTLVTVSS | |
| | VH-DNA sequence | SEQ ID |
| | CAGGTCCAGCTTGTGCAGTCTGGGGCTGAGGTGAAGAAGCC | NO: 63 |
| | TGGGGCCTCAGTGAAGGTTTCCTGCAAGGCTTCTGGATACAC | |
| | CTTCGACAGCTATAAAATGCATTGGGTGCGCCAGGCCCCCGG | |
| | ACAAAGGCTTGAGTGGATGGGATACATCCCCGCTGGCACTG | |
| | GTAACACAAAATATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACCGTGGCGGATACTACTACGACAGCTGGGATGAT | |
| | TGGGGACAGGGTACATTGGTCACCGTCTCCTCA | |
| | Heavy chain-Amino acid sequence | SEQ ID |
| | QVQLVQSGAEVKKPGASVKVSCKASGYTFDSYKMHWVRQAP | NO: 64 |
| | GQRLEWMGYIPAGTGNTKYSQKFQGRVTITRDTSASTAYMELS | |
| | SLRSEDTAVYYCARDRGGYYYDSWDDWGQGTLVTVSSASTK | |
| | GPSVFPLAPSSKSTSGGTAALGCLVKDYFPEPVTVSWNSGALTS | |
| | GVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSC | |
| | SVMHEALHNHYTQKSLSLSPGK | |
| | Heavy chain-DNA sequence | SEQ ID |
| | CAGGTCCAGCTTGTGCAGTCTGGGGCTGAGGTGAAGAAGCC | NO: 65 |
| | TGGGGCCTCAGTGAAGGTTTCCTGCAAGGCTTCTGGATACAC | |
| | CTTCGACAGCTATAAAATGCATTGGGTGCGCCAGGCCCCCGG | |
| | ACAAAGGCTTGAGTGGATGGGATACATCCCCGCTGGCACTG | |
| | GTAACACAAAATATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACCGTGGCGGATACTACTACGACAGCTGGGATGAT | |
| | TGGGGACAGGGTACATTGGTCACCGTCTCCTCAGCCTCCACC | |
| | AAGGGCCCTTCTGTGTTCCCCCTGGCGCCTTCCAGTAAGTCC | |
| | ACCTCCGGAGGCACAGCCGCTTTGGGCTGTCTGGTCAAGGAC | |
| | TACTTTCCTGAGCCCGTGACCGTCTCCTGGAACTCAGGAGCC | |
| | CTGACCAGTGGGGTACATACGTTTCCAGCCGTCTTGCAGTCC | |
| | TCTGGACTGTACTCTCTCAGCTCTGTAGTGACCGTGCCCAGC | |
| | AGTTCCCTGGGCACCCAGACATACATTTGCAACGTCAATCAC | |
| | AAGCCGTCTAACACCAAGGTAGATAAAAAGGTGGAGCCTAA | |
| | GTCTTGCGATAAGACCCATACCTGCCCGCCCTGCCCCGCCCC | |
| | AGAGCTGCTCGGGGGACCGAGCGTCTTCCTCTTCCCACCCAA | |
| | GCCCAAGGATACCCTGATGATCTCCCGCACTCCCGAAGTGAC | |
| | CTGTGTGGTTGTGGACGTCTCCCATGAGGACCCTGAGGTTAA | |
| | GTTTAACTGGTACGTAGATGGCGTTGAGGTTCACAATGCCAA | |
| | GACCAAGCCGCGTGAAGAGCAGTATAACTCCACCTACAGGG | |
| | TCGTGAGCGTCCTGACCGTGCTTCATCAGGATTGGCTGAACG | |
| | GGAAGGAGTATAAGTGCAAAGTCAGCAACAAGGCCCTGCCC | |
| | GCACCTATCGAGAAGACCATTAGCAAGGCCAAGGGTCAGCC | |
| | TCGCGAGCCCCAGGTGTACACCCTGCCGCCCTCCCGTGACGA | |
| | GCTCACAAAGAACCAGGTGTCCTTGACCTGCCTGGTTAAAGG | |
| | ATTCTACCCGTCAGATATTGCCGTGGAATGGGAGAGCAATG | |
| | GCCAGCCTGAGAATAACTACAAGACAACTCCCCCGGTGCTC | |
| | GACTCCGACGGCAGTTTTTTCCTGTATTCAAAGCTCACAGTG | |
| | GATAAGTCTAGGTGGCAACAGGGTAATGTTTTCAGCTGTTCC | |
| | GTTATGCACGAGGCCCTCCACAACCACTACACCCAGAAATC | |
| | CCTGAGCCTGTCCCCAGGGAAG | |
| | Light Chain | |
| | LCDR1 (IMGT) | SEQ ID |
| | QSISSY | NO: 66 |
| | LCDR2 (IMGT) | SEQ ID |
| | GA | NO: 67 |
| | LCDR3 (IMGT) | SEQ ID |
| | QQVAFTPPT | NO: 68 |
| | LCDR1 (Kabat) | SEQ ID |
| | RASQSISSYLN | NO: 69 |
| | LCDR2 (Kabat) | SEQ ID |
| | GASSLQS | NO: 70 |
| | LCDR3 (Kabat) | SEQ ID |
| | QQVAFTPPT | NO: 71 |
| | LCDR1 (Combined Kabat/IMGT) | SEQ ID |
| | RASQSISSYLN | NO: 72 |
| | LCDR2 (Combined Kabat/IMGT) | SEQ ID |
| | GASSLQS | NO: 73 |
| | LCDR3 (Combined Kabat/IMGT) | SEQ ID |
| | QQVAFTPPT | NO: 74 |
| | VL-Amino acid sequence | SEQ ID |
| | DIQMTQSPSSLSASVGDRVTITCRASQSISSYLNWYQQKPGKAP | NO: 75 |
| | KLLIYGASSLQSGVPSRFSGSGSGTDFTLTISSLQPEDFATYYCQ | |
| | QVAFTPPTFGGGTKVEIK | |
| | VL-DNA sequence | SEQ ID |
| | GACATCCAGATGACCCAGTCTCCATCCTCCCTGTCTGCATCT | NO: 76 |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTAGCCTTCACTCCTCCTACT | |
| | TTTGGCGGAGGGACCAAGGTTGAGATCAAA | |
| | Light chain-Amino acid sequence | SEQ ID |
| | DIQMTQSPSSLSASVGDRVTITCRASQSISSYLNWYQQKPGKAP | NO: 77 |
| | KLLIYGASSLQSGVPSRFSGSGSGTDFTLTISSLQPEDFATYYCQ | |
| | QVAFTPPTFGGGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | Light chain-DNA sequence | SEQ ID |
| | GACATCCAGATGACCCAGTCTCCATCCTCCCTGTCTGCATCT | NO: 78 |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTAGCCTTCACTCCTCCTACT | |
| | TTTGGCGGAGGGACCAAGGTTGAGATCAAACGTACGGTGGC | |
| | TGCACCATCTGTCTTCATCTTCCCGCCATCTGATGAGCAGTTG | |
| | AAATCTGGAACTGCCTCTGTTGTGTGCCTGCTGAATAACTTC | |
| | TATCCCAGAGAGGCCAAAGTACAGTGGAAGGTGGATAACGC | |
| | CCTCCAATCGGGTAACTCCCAGGAGAGTGTCACAGAGCAGG | |
| | ACAGCAAGGACAGCACCTACAGCCTCAGCAGCACCCTGACG | |
| | CTGAGCAAAGCAGACTACGAGAAACACAAAGTCTACGCCTG | |
| | CGAAGTCACCCATCAGGGCCTGAGCTCGCCCGTCACAAAGA | |
| | GCTTCAACAGGGGAGAGTGT | |
| Ab- | Heavy Chain | |
| 65886 | HCDR1 (IMGT) | SEQ ID |
| | GYTFEKYA | NO: 79 |
| | HCDR2 (IMGT) | SEQ ID |
| | IPAGTGNT | NO: 80 |
| | HCDR3 (IMGT) | SEQ ID |
| | ARDRGGYYYDSWDD | NO: 81 |
| | HCDR1 (Kabat) | SEQ ID |
| | KYAMH | NO: 82 |
| | HCDR2 (Kabat) | SEQ ID |
| | FIPAGTGNTKYSQKFQG | NO: 83 |
| | HCDR3 (Kabat) | SEQ ID |
| | DRGGYYYDSWDD | NO: 84 |
| | HCDR1 (Combined Kabat/IMGT) | SEQ ID |
| | GYTFEKYAMH | NO: 85 |
| | HCDR2 (Combined Kabat/IMGT) | SEQ ID |
| | FIPAGTGNTKYSQKFQG | NO: 86 |
| | HCDR3 (Combined Kabat/IMGT) | SEQ ID |
| | DRGGYYYDSWDD | NO: 87 |
| | VH-Amino acid sequence | SEQ ID |
| | QVQLVQSGAEVKKPGASVKVSCKASGYTFEKYAMHWVRQAP | NO: 88 |
| | GQRLEWMGFIPAGTGNTKYSQKFQGRVTITRDTSASTAYMELS | |
| | SLRSEDTAVYYCARDRGGYYYDSWDDWGQGTLVTVSS | |
| | VH-DNA sequence | SEQ ID |
| | CAGGTCCAGCTTGTGCAGTCTGGGGCTGAGGTGAAGAAGCC | NO: 89 |
| | TGGGGCCTCAGTGAAGGTTTCCTGCAAGGCTTCTGGATACAC | |
| | CTTCGAAAAATATGCTATGCATTGGGTGCGCCAGGCCCCCGG | |
| | ACAAAGGCTTGAGTGGATGGGATTCATCCCCGCTGGCACTG | |
| | GTAACACAAAATATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACCGTGGCGGATACTACTACGACAGCTGGGATGAT | |
| | TGGGGACAGGGTACATTGGTCACCGTCTCCTCA | |
| | Heavy chain-Amino acid sequence | SEQ ID |
| | QVQLVQSGAEVKKPGASVKVSCKASGYTFEKYAMHWVRQAP | NO: 90 |
| | GQRLEWMGFIPAGTGNTKYSQKFQGRVTITRDTSASTAYMELS | SEQ ID |
| | SLRSEDTAVYYCARDRGGYYYDSWDDWGQGTLVTVSSASTK | NO: 91 |
| | GPSVFPLAPSSKSTSGGTAALGCLVKDYFPEPVTVSWNSGALTS | |
| | GVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSC | |
| | SVMHEALHNHYTQKSLSLSPGK | |
| | Heavy chain-DNA sequence | |
| | CAGGTCCAGCTTGTGCAGTCTGGGGCTGAGGTGAAGAAGCC | |
| | TGGGGCCTCAGTGAAGGTTTCCTGCAAGGCTTCTGGATACAC | |
| | CTTCGAAAAATATGCTATGCATTGGGTGCGCCAGGCCCCCGG | |
| | ACAAAGGCTTGAGTGGATGGGATTCATCCCCGCTGGCACTG | |
| | GTAACACAAAATATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACCGTGGCGGATACTACTACGACAGCTGGGATGAT | |
| | TGGGGACAGGGTACATTGGTCACCGTCTCCTCAGCCTCCACC | |
| | AAGGGCCCTTCTGTGTTCCCCCTGGCGCCTTCCAGTAAGTCC | |
| | ACCTCCGGAGGCACAGCCGCTTTGGGCTGTCTGGTCAAGGAC | |
| | TACTTTCCTGAGCCCGTGACCGTCTCCTGGAACTCAGGAGCC | |
| | CTGACCAGTGGGGTACATACGTTTCCAGCCGTCTTGCAGTCC | |
| | TCTGGACTGTACTCTCTCAGCTCTGTAGTGACCGTGCCCAGC | |
| | AGTTCCCTGGGCACCCAGACATACATTTGCAACGTCAATCAC | |
| | AAGCCGTCTAACACCAAGGTAGATAAAAAGGTGGAGCCTAA | |
| | GTCTTGCGATAAGACCCATACCTGCCCGCCCTGCCCCGCCCC | |
| | AGAGCTGCTCGGGGGACCGAGCGTCTTCCTCTTCCCACCCAA | |
| | GCCCAAGGATACCCTGATGATCTCCCGCACTCCCGAAGTGAC | |
| | CTGTGTGGTTGTGGACGTCTCCCATGAGGACCCTGAGGTTAA | |
| | GTTTAACTGGTACGTAGATGGCGTTGAGGTTCACAATGCCAA | |
| | GACCAAGCCGCGTGAAGAGCAGTATAACTCCACCTACAGGG | |
| | TCGTGAGCGTCCTGACCGTGCTTCATCAGGATTGGCTGAACG | |
| | GGAAGGAGTATAAGTGCAAAGTCAGCAACAAGGCCCTGCCC | |
| | GCACCTATCGAGAAGACCATTAGCAAGGCCAAGGGTCAGCC | |
| | TCGCGAGCCCCAGGTGTACACCCTGCCGCCCTCCCGTGACGA | |
| | GCTCACAAAGAACCAGGTGTCCTTGACCTGCCTGGTTAAAGG | |
| | ATTCTACCCGTCAGATATTGCCGTGGAATGGGAGAGCAATG | |
| | GCCAGCCTGAGAATAACTACAAGACAACTCCCCCGGTGCTC | |
| | GACTCCGACGGCAGTTTTTTCCTGTATTCAAAGCTCACAGTG | |
| | GATAAGTCTAGGTGGCAACAGGGTAATGTTTTCAGCTGTTCC | |
| | GTTATGCACGAGGCCCTCCACAACCACTACACCCAGAAATC | |
| | CCTGAGCCTGTCCCCAGGGAAG | |
| | Light Chain | |
| | LCDR1 (IMGT) | SEQ ID |
| | QSISSY | NO: 92 |
| | LCDR2 (IMGT) | SEQ ID |
| | GA | NO: 93 |
| | LCDR3 (IMGT) | SEQ ID |
| | QQVAFTPPT | NO: 94 |
| | LCDR1 (Kabat) | SEQ ID |
| | RASQSISSYLN | NO: 95 |
| | LCDR2 (Kabat) | SEQ ID |
| | GASSLQS | NO: 96 |
| | LCDR3 (Kabat) | SEQ ID |
| | QQVAFTPPT | NO: 97 |
| | LCDR1 (Combined Kabat/IMGT) | SEQ ID |
| | RASQSISSYLN | NO: 98 |
| | LCDR2 (Combined Kabat/IMGT) | SEQ ID |
| | GASSLQS | NO: 99 |
| | LCDR3 (Combined Kabat/IMGT) | SEQ ID |
| | QQVAFTPPT | NO: 100 |
| | VL-Amino acid sequence | SEQ ID |
| | DIQMTQSPSSLSASVGDRVTITCRASQSISSYLNWYQQKPGKAP | NO: 101 |
| | KLLIYGASSLQSGVPSRFSGSGSGTDFTLTISSLQPEDFATYYCQ | |
| | QVAFTPPTFGGGTKVEIK | |
| | VL-DNA sequence | SEQ ID |
| | GACATCCAGATGACCCAGTCTCCATCCTCCCTGTCTGCATCT | NO: 102 |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTAGCCTTCACTCCTCCTACT | |
| | TTTGGCGGAGGGACCAAGGTTGAGATCAAA | |
| | Light chain-Amino acid sequence | SEQ ID |
| | DIQMTQSPSSLSASVGDRVTITCRASQSISSYLNWYQQKPGKAP | NO: 103 |
| | KLLIYGASSLQSGVPSRFSGSGSGTDFTLTISSLQPEDFATYYCQ | |
| | QVAFTPPTFGGGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | Light chain-DNA sequence | SEQ ID |
| | GACATCCAGATGACCCAGTCTCCATCCTCCCTGTCTGCATCT | NO: 104 |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTAGCCTTCACTCCTCCTACT | |
| | TTTGGCGGAGGGACCAAGGTTGAGATCAAACGTACGGTGGC | |
| | TGCACCATCTGTCTTCATCTTCCCGCCATCTGATGAGCAGTTG | |
| | AAATCTGGAACTGCCTCTGTTGTGTGCCTGCTGAATAACTTC | |
| | TATCCCAGAGAGGCCAAAGTACAGTGGAAGGTGGATAACGC | |
| | CCTCCAATCGGGTAACTCCCAGGAGAGTGTCACAGAGCAGG | |
| | ACAGCAAGGACAGCACCTACAGCCTCAGCAGCACCCTGACG | |
| | CTGAGCAAAGCAGACTACGAGAAACACAAAGTCTACGCCTG | |
| | CGAAGTCACCCATCAGGGCCTGAGCTCGCCCGTCACAAAGA | |
| | GCTTCAACAGGGGAGAGTGT | |
| Ab- | Heavy Chain | |
| 65887 | HCDR1 (IMGT) | SEQ ID |
| | GYTFEQYA | NO: 105 |
| | HCDR2 (IMGT) | SEQ ID |
| | IPAGTGNT | NO: 106 |
| | HCDR3 (IMGT) | SEQ ID |
| | ARDRGGYWYDSWDD | NO: 107 |
| | HCDR1 (Kabat) | SEQ ID |
| | QYAMH | NO: 108 |
| | HCDR2 (Kabat) | SEQ ID |
| | FIPAGTGNTKYSQKFQG | NO: 109 |
| | HCDR3 (Kabat) | SEQ ID |
| | DRGGYWYDSWDD | NO: 110 |
| | HCDR1 (Combined Kabat/IMGT) | SEQ ID |
| | GYTFEQYAMH | NO: 111 |
| | HCDR2 (Combined Kabat/IMGT) | SEQ ID |
| | FIPAGTGNTKYSQKFQG | NO: 112 |
| | HCDR3 (Combined Kabat/IMGT) | SEQ ID |
| | DRGGYWYDSWDD | NO: 113 |
| | VH-Amino acid sequence | SEQ ID |
| | QVQLVQSGAEVKKPGASVKVSCKASGYTFEQYAMHWVRQAP | NO: 114 |
| | GQRLEWMGFIPAGTGNTKYSQKFQGRVTITRDTSASTAYMELS | |
| | SLRSEDTAVYYCARDRGGYWYDSWDDWGQGTLVTVSS | |
| | VH-DNA sequence | SEQ ID |
| | CAGGTCCAGCTTGTGCAGTCTGGGGCTGAGGTGAAGAAGCC | NO: 115 |
| | TGGGGCCTCAGTGAAGGTTTCCTGCAAGGCTTCTGGATACAC | |
| | CTTCGAACAATATGCTATGCATTGGGTGCGCCAGGCCCCCGG | |
| | ACAAAGGCTTGAGTGGATGGGATTCATCCCCGCTGGCACTG | |
| | GTAACACAAAATATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACCGTGGCGGATACTGGTACGACAGTTGGGATGAT | |
| | TGGGGACAGGGTACATTGGTCACCGTCTCCTCA | |
| | Heavy chain-Amino acid sequence | SEQ ID |
| | QVQLVQSGAEVKKPGASVKVSCKASGYTFEQYAMHWVRQAP | NO: 116 |
| | GQRLEWMGFIPAGTGNTKYSQKFQGRVTITRDTSASTAYMELS | |
| | SLRSEDTAVYYCARDRGGYWYDSWDDWGQGTLVTVSSASTK | |
| | GPSVFPLAPSSKSTSGGTAALGCLVKDYFPEPVTVSWNSGALTS | |
| | GVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSC | |
| | SVMHEALHNHYTQKSLSLSPGK | |
| | Heavy chain-DNA sequence | SEQ ID |
| | CAGGTCCAGCTTGTGCAGTCTGGGGCTGAGGTGAAGAAGCC | NO: 117 |
| | TGGGGCCTCAGTGAAGGTTTCCTGCAAGGCTTCTGGATACAC | |
| | CTTCGAACAATATGCTATGCATTGGGTGCGCCAGGCCCCCGG | |
| | ACAAAGGCTTGAGTGGATGGGATTCATCCCCGCTGGCACTG | |
| | GTAACACAAAATATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACCGTGGCGGATACTGGTACGACAGTTGGGATGAT | |
| | TGGGGACAGGGTACATTGGTCACCGTCTCCTCAGCCTCCACC | |
| | AAGGGCCCTTCTGTGTTCCCCCTGGCGCCTTCCAGTAAGTCC | |
| | ACCTCCGGAGGCACAGCCGCTTTGGGCTGTCTGGTCAAGGAC | |
| | TACTTTCCTGAGCCCGTGACCGTCTCCTGGAACTCAGGAGCC | |
| | CTGACCAGTGGGGTACATACGTTTCCAGCCGTCTTGCAGTCC | |
| | TCTGGACTGTACTCTCTCAGCTCTGTAGTGACCGTGCCCAGC | |
| | AGTTCCCTGGGCACCCAGACATACATTTGCAACGTCAATCAC | |
| | AAGCCGTCTAACACCAAGGTAGATAAAAAGGTGGAGCCTAA | |
| | GTCTTGCGATAAGACCCATACCTGCCCGCCCTGCCCCGCCCC | |
| | AGAGCTGCTCGGGGGACCGAGCGTCTTCCTCTTCCCACCCAA | |
| | GCCCAAGGATACCCTGATGATCTCCCGCACTCCCGAAGTGAC | |
| | CTGTGTGGTTGTGGACGTCTCCCATGAGGACCCTGAGGTTAA | |
| | GTTTAACTGGTACGTAGATGGCGTTGAGGTTCACAATGCCAA | |
| | GACCAAGCCGCGTGAAGAGCAGTATAACTCCACCTACAGGG | |
| | TCGTGAGCGTCCTGACCGTGCTTCATCAGGATTGGCTGAACG | |
| | GGAAGGAGTATAAGTGCAAAGTCAGCAACAAGGCCCTGCCC | |
| | GCACCTATCGAGAAGACCATTAGCAAGGCCAAGGGTCAGCC | |
| | TCGCGAGCCCCAGGTGTACACCCTGCCGCCCTCCCGTGACGA | |
| | GCTCACAAAGAACCAGGTGTCCTTGACCTGCCTGGTTAAAGG | |
| | ATTCTACCCGTCAGATATTGCCGTGGAATGGGAGAGCAATG | |
| | GCCAGCCTGAGAATAACTACAAGACAACTCCCCCGGTGCTC | |
| | GACTCCGACGGCAGTTTTTTCCTGTATTCAAAGCTCACAGTG | |
| | GATAAGTCTAGGTGGCAACAGGGTAATGTTTTCAGCTGTTCC | |
| | GTTATGCACGAGGCCCTCCACAACCACTACACCCAGAAATC | |
| | CCTGAGCCTGTCCCCAGGGAAG | |
| | Light Chain | |
| | LCDR1 (IMGT) | SEQ ID |
| | QSISSY | NO: 118 |
| | LCDR2 (IMGT) | SEQ ID |
| | GA | NO: 119 |
| | LCDR3 (IMGT) | SEQ ID |
| | QQVVFTPPT | NO: 120 |
| | LCDR1 (Kabat) | SEQ ID |
| | RASQSISSYLN | NO: 121 |
| | LCDR2 (Kabat) | SEQ ID |
| | GASSLQS | NO: 122 |
| | LCDR3 (Kabat) | SEQ ID |
| | QQVVFTPPT | NO: 123 |
| | LCDR1 (Combined Kabat/IMGT) | SEQ ID |
| | RASQSISSYLN | NO: 124 |
| | LCDR2 (Combined Kabat/IMGT) | SEQ ID |
| | GASSLQS | NO: 125 |
| | LCDR3 (Combined Kabat/IMGT) | SEQ ID |
| | QQVVFTPPT | NO: 126 |
| | VL-Amino acid sequence | SEQ ID |
| | DIQMTQSPSSLSASVGDRVTITCRASQSISSYLNWYQQKPGKAP | NO: 127 |
| | KLLIYGASSLQSGVPSRFSGSGSGTDFTLTISSLQPEDFATYYCQ | |
| | QVVFTPPTFGGGTKVEIK | |
| | VL-DNA sequence | SEQ ID |
| | GACATCCAGATGACCCAGTCTCCATCCTCCCTGTCTGCATCT | NO: 128 |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTAGTGTTCACGCCTCCTACT | |
| | TTTGGCGGAGGGACCAAGGTTGAGATCAAA | |
| | Light chain-Amino acid sequence | SEQ ID |
| | DIQMTQSPSSLSASVGDRVTITCRASQSISSYLNWYQQKPGKAP | NO: 129 |
| | KLLIYGASSLQSGVPSRFSGSGSGTDFTLTISSLQPEDFATYYCQ | |
| | QVVFTPPTFGGGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | Light chain-DNA sequence | SEQ ID |
| | GACATCCAGATGACCCAGTCTCCATCCTCCCTGTCTGCATCT | NO: 130 |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTAGTGTTCACGCCTCCTACT | |
| | TTTGGCGGAGGGACCAAGGTTGAGATCAAACGTACGGTGGC | |
| | TGCACCATCTGTCTTCATCTTCCCGCCATCTGATGAGCAGTTG | |
| | AAATCTGGAACTGCCTCTGTTGTGTGCCTGCTGAATAACTTC | |
| | TATCCCAGAGAGGCCAAAGTACAGTGGAAGGTGGATAACGC | |
| | CCTCCAATCGGGTAACTCCCAGGAGAGTGTCACAGAGCAGG | |
| | ACAGCAAGGACAGCACCTACAGCCTCAGCAGCACCCTGACG | |
| | CTGAGCAAAGCAGACTACGAGAAACACAAAGTCTACGCCTG | |
| | CGAAGTCACCCATCAGGGCCTGAGCTCGCCCGTCACAAAGA | |
| | GCTTCAACAGGGGAGAGTGT | |
| Ab- | Heavy Chain | |
| 65889 | HCDR1 (IMGT) | SEQ ID |
| | GYTFASYK | NO: 131 |
| | HCDR2 (IMGT) | SEQ ID |
| | IPAGTGNT | NO: 132 |
| | HCDR3 (IMGT) | SEQ ID |
| | ARDRGGYYYDSWDD | NO: 133 |
| | HCDR1 (Kabat) | SEQ ID |
| | SYKMH | NO: 134 |
| | HCDR2 (Kabat) | SEQ ID |
| | DIPAGTGNTHYSQKFQG | NO: 135 |
| | HCDR3 (Kabat) | SEQ ID |
| | DRGGYYYDSWDD | NO: 136 |
| | HCDR1 (Combined Kabat/IMGT) | SEQ ID |
| | GYTFASYKMH | NO: 137 |
| | HCDR2 (Combined Kabat/IMGT) | SEQ ID |
| | DIPAGTGNTHYSQKFQG | NO: 138 |
| | HCDR3 (Combined Kabat/IMGT) | SEQ ID |
| | DRGGYYYDSWDD | NO: 139 |
| | V-Amino acid sequence | SEQ ID |
| | QVQLVQSGAEVKKPGASVKVSCKASGYTFASYKMHWVRQAP | NO: 140 |
| | GQRLEWMGDIPAGTGNTHYSQKFQGRVTITRDTSASTAYMELS | |
| | SLRSEDTAVYYCARDRGGYYYDSWDDWGQGTLVTVSS | |
| | VH-DNA sequence | SEQ ID |
| | CAGGTCCAGCTTGTGCAGTCTGGGGCTGAGGTGAAGAAGCC | NO: 141 |
| | TGGGGCCTCAGTGAAGGTTTCCTGCAAGGCTTCTGGATACAC | |
| | CTTCGCCAGCTATAAAATGCATTGGGTGCGCCAGGCCCCCGG | |
| | ACAAAGGCTTGAGTGGATGGGAGACATCCCCGCTGGCACTG | |
| | GTAACACACACTATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACCGTGGGGGATACTACTACGATAGCTGGGATGAT | |
| | TGGGGACAGGGTACATTGGTCACCGTCTCCTCA | |
| | Heavy chain-Amino acid sequence | SEQ ID |
| | QVQLVQSGAEVKKPGASVKVSCKASGYTFASYKMHWVRQAP | NO: 142 |
| | GQRLEWMGDIPAGTGNTHYSQKFQGRVTITRDTSASTAYMELS | |
| | SLRSEDTAVYYCARDRGGYYYDSWDDWGQGTLVTVSSASTK | |
| | GPSVFPLAPSSKSTSGGTAALGCLVKDYFPEPVTVSWNSGALTS | |
| | GVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSC | |
| | SVMHEALHNHYTQKSLSLSPGK | |
| | Heavy chain-DNA sequence | SEQ ID |
| | CAGGTCCAGCTTGTGCAGTCTGGGGCTGAGGTGAAGAAGCC | NO: 143 |
| | TGGGGCCTCAGTGAAGGTTTCCTGCAAGGCTTCTGGATACAC | |
| | CTTCGCCAGCTATAAAATGCATTGGGTGCGCCAGGCCCCCGG | |
| | ACAAAGGCTTGAGTGGATGGGAGACATCCCCGCTGGCACTG | |
| | GTAACACACACTATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACCGTGGGGGATACTACTACGATAGCTGGGATGAT | |
| | TGGGGACAGGGTACATTGGTCACCGTCTCCTCAGCCTCCACC | |
| | AAGGGCCCTTCTGTGTTCCCCCTGGCGCCTTCCAGTAAGTCC | |
| | ACCTCCGGAGGCACAGCCGCTTTGGGCTGTCTGGTCAAGGAC | |
| | TACTTTCCTGAGCCCGTGACCGTCTCCTGGAACTCAGGAGCC | |
| | CTGACCAGTGGGGTACATACGTTTCCAGCCGTCTTGCAGTCC | |
| | TCTGGACTGTACTCTCTCAGCTCTGTAGTGACCGTGCCCAGC | |
| | AGTTCCCTGGGCACCCAGACATACATTTGCAACGTCAATCAC | |
| | AAGCCGTCTAACACCAAGGTAGATAAAAAGGTGGAGCCTAA | |
| | GTCTTGCGATAAGACCCATACCTGCCCGCCCTGCCCCGCCCC | |
| | AGAGCTGCTCGGGGGACCGAGCGTCTTCCTCTTCCCACCCAA | |
| | GCCCAAGGATACCCTGATGATCTCCCGCACTCCCGAAGTGAC | |
| | CTGTGTGGTTGTGGACGTCTCCCATGAGGACCCTGAGGTTAA | |
| | GTTTAACTGGTACGTAGATGGCGTTGAGGTTCACAATGCCAA | |
| | GACCAAGCCGCGTGAAGAGCAGTATAACTCCACCTACAGGG | |
| | TCGTGAGCGTCCTGACCGTGCTTCATCAGGATTGGCTGAACG | |
| | GGAAGGAGTATAAGTGCAAAGTCAGCAACAAGGCCCTGCCC | |
| | GCACCTATCGAGAAGACCATTAGCAAGGCCAAGGGTCAGCC | |
| | TCGCGAGCCCCAGGTGTACACCCTGCCGCCCTCCCGTGACGA | |
| | GCTCACAAAGAACCAGGTGTCCTTGACCTGCCTGGTTAAAGG | |
| | ATTCTACCCGTCAGATATTGCCGTGGAATGGGAGAGCAATG | |
| | GCCAGCCTGAGAATAACTACAAGACAACTCCCCCGGTGCTC | |
| | GACTCCGACGGCAGTTTTTTCCTGTATTCAAAGCTCACAGTG | |
| | GATAAGTCTAGGTGGCAACAGGGTAATGTTTTCAGCTGTTCC | |
| | GTTATGCACGAGGCCCTCCACAACCACTACACCCAGAAATC | |
| | CCTGAGCCTGTCCCCAGGGAAG | |
| | Light Chain | |
| | LCDR1 (IMGT) | SEQ ID |
| | QSISSY | NO: 144 |
| | LCDR2 (IMGT) | SEQ ID |
| | GA | NO: 145 |
| | LCDR3 (IMGT) | SEQ ID |
| | QQVPFEPPT | NO: 146 |
| | LCDR1 (Kabat) | SEQ ID |
| | RASQSISSYLN | NO: 147 |
| | LCDR2 (Kabat) | SEQ ID |
| | GASSLQS | NO: 148 |
| | LCDR3 (Kabat) | SEQ ID |
| | QQVPFEPPT | NO: 149 |
| | LCDR1 (Combined Kabat/IMGT) | SEQ ID |
| | RASQSISSYLN | NO: 150 |
| | LCDR2 (Combined Kabat/IMGT) | SEQ ID |
| | GASSLQS | NO: 151 |
| | LCDR3 (Combined Kabat/IMGT) | SEQ ID |
| | QQVPFEPPT | NO: 152 |
| | VL-Amino acid sequence | SEQ ID |
| | DIQMTQSPSSLSASVGDRVTITCRASQSISSYLNWYQQKPGKAP | NO: 153 |
| | KLLIYGASSLQSGVPSRFSGSGSGTDFTLTISSLQPEDFATYYCQ | |
| | QVPFEPPTFGGGTKVEIK | |
| | VL-DNA sequence | SEQ ID |
| | GACATCCAGATGACCCAGTCTCCATCCTCCCTGTCTGCATCT | NO: 154 |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTACCGTTCGAGCCTCCTACT | |
| | TTTGGCGGAGGGACCAAGGTTGAGATCAAA | |
| | Light chain-Amino acid sequence | SEQ ID |
| | DIQMTQSPSSLSASVGDRVTITCRASQSISSYLNWYQQKPGKAP | NO: 155 |
| | KLLIYGASSLQSGVPSRFSGSGSGTDFTLTISSLQPEDFATYYCQ | |
| | QVPFEPPTFGGGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | Light chain-DNA sequence | SEQ ID |
| | GACATCCAGATGACCCAGTCTCCATCCTCCCTGTCTGCATCT | NO: 156 |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTACCGTTCGAGCCTCCTACT | |
| | TTTGGCGGAGGGACCAAGGTTGAGATCAAACGTACGGTGGC | |
| | TGCACCATCTGTCTTCATCTTCCCGCCATCTGATGAGCAGTTG | |
| | AAATCTGGAACTGCCTCTGTTGTGTGCCTGCTGAATAACTTC | |
| | TATCCCAGAGAGGCCAAAGTACAGTGGAAGGTGGATAACGC | |
| | CCTCCAATCGGGTAACTCCCAGGAGAGTGTCACAGAGCAGG | |
| | ACAGCAAGGACAGCACCTACAGCCTCAGCAGCACCCTGACG | |
| | CTGAGCAAAGCAGACTACGAGAAACACAAAGTCTACGCCTG | |
| | CGAAGTCACCCATCAGGGCCTGAGCTCGCCCGTCACAAAGA | |
| | GCTTCAACAGGGGAGAGTGT | |
| Ab- | Heavy Chain | |
| 65890 | HCDR1 (IMGT) | SEQ ID |
| | GYTFNSYR | NO: 157 |
| | HCDR2 (IMGT) | SEQ ID |
| | IPAGTGNT | NO: 158 |
| | HCDR3 (IMGT) | SEQ ID |
| | ARDRGGYYYDSWDD | NO: 159 |
| | HCDR1 (Kabat) | SEQ ID |
| | SYRMH | NO: 160 |
| | HCDR2 (Kabat) | SEQ ID |
| | SIPAGTGNTVYSQKFQG | NO: 161 |
| | HCDR3 (Kabat) | SEQ ID |
| | DRGGYYYDSWDD | NO: 162 |
| | HCDR1 (Combined Kabat/IMGT) | SEQ ID |
| | GYTFNSYRMH | NO: 163 |
| | HCDR2 (Combined Kabat/IMGT) | SEQ ID |
| | SIPAGTGNTVYSQKFQG | NO: 164 |
| | HCDR3 (Combined Kabat/IMGT) | SEQ ID |
| | DRGGYYYDSWDD | NO: 165 |
| | VH-Amino acid sequence | SEQ ID |
| | QVQLVQSGAEVKKPGASVKVSCKASGYTFNSYRMHWVRQAP | NO: 166 |
| | GQRLEWMGSIPAGTGNTVYSQKFQGRVTITRDTSASTAYMELS | |
| | SLRSEDTAVYYCARDRGGYYYDSWDDWGQGTLVTVSS | |
| | VH-DNA sequence | SEQ ID |
| | CAGGTCCAGCTTGTGCAGTCTGGGGCTGAGGTGAAGAAGCC | NO: 167 |
| | TGGGGCCTCAGTGAAGGTTTCCTGCAAGGCTTCTGGATACAC | |
| | CTTCAACAGCTATCGAATGCATTGGGTGCGCCAGGCCCCCGG | |
| | ACAAAGGCTTGAGTGGATGGGATCCATCCCCGCTGGCACTG | |
| | GTAACACAGTCTATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACAGGGGGGGGTACTACTACGACAGCTGGGATGAT | |
| | TGGGGACAGGGTACATTGGTCACCGTCTCCTCA | |
| | Heavy chain-Amino acid sequence | SEQ ID |
| | QVQLVQSGAEVKKPGASVKVSCKASGYTFNSYRMHWVRQAP | NO: 168 |
| | GQRLEWMGSIPAGTGNTVYSQKFQGRVTITRDTSASTAYMELS | |
| | SLRSEDTAVYYCARDRGGYYYDSWDDWGQGTLVTVSSASTK | |
| | GPSVFPLAPSSKSTSGGTAALGCLVKDYFPEPVTVSWNSGALTS | |
| | GVHTFPAVLQSSGLYSLSSVVTVPSSSLGTQTYICNVNHKPSNT | |
| | KVDKKVEPKSCDKTHTCPPCPAPELLGGPSVFLFPPKPKDTLMI | |
| | SRTPEVTCVVVDVSHEDPEVKFNWYVDGVEVHNAKTKPREEQ | |
| | YNSTYRVVSVLTVLHQDWLNGKEYKCKVSNKALPAPIEKTISK | |
| | AKGQPREPQVYTLPPSRDELTKNQVSLTCLVKGFYPSDIAVEWE | |
| | SNGQPENNYKTTPPVLDSDGSFFLYSKLTVDKSRWQQGNVFSC | |
| | SVMHEALHNHYTQKSLSLSPGK | |
| | Heavy chain-DNA sequence | SEQ ID |
| | CAGGTCCAGCTTGTGCAGTCTGGGGCTGAGGTGAAGAAGCC | NO: 169 |
| | TGGGGCCTCAGTGAAGGTTTCCTGCAAGGCTTCTGGATACAC | |
| | CTTCAACAGCTATCGAATGCATTGGGTGCGCCAGGCCCCCGG | |
| | ACAAAGGCTTGAGTGGATGGGATCCATCCCCGCTGGCACTG | |
| | GTAACACAGTCTATTCACAGAAGTTCCAGGGCAGAGTCACC | |
| | ATTACCAGGGACACATCCGCGAGCACAGCCTACATGGAGCT | |
| | GAGCAGCCTGAGATCTGAAGACACGGCGGTGTACTACTGCG | |
| | CCAGAGACAGGGGGGGGTACTACTACGACAGCTGGGATGAT | |
| | TGGGGACAGGGTACATTGGTCACCGTCTCCTCAGCCTCCACC | |
| | AAGGGCCCTTCTGTGTTCCCCCTGGCGCCTTCCAGTAAGTCC | |
| | ACCTCCGGAGGCACAGCCGCTTTGGGCTGTCTGGTCAAGGAC | |
| | TACTTTCCTGAGCCCGTGACCGTCTCCTGGAACTCAGGAGCC | |
| | CTGACCAGTGGGGTACATACGTTTCCAGCCGTCTTGCAGTCC | |
| | TCTGGACTGTACTCTCTCAGCTCTGTAGTGACCGTGCCCAGC | |
| | AGTTCCCTGGGCACCCAGACATACATTTGCAACGTCAATCAC | |
| | AAGCCGTCTAACACCAAGGTAGATAAAAAGGTGGAGCCTAA | |
| | GTCTTGCGATAAGACCCATACCTGCCCGCCCTGCCCCGCCCC | |
| | AGAGCTGCTCGGGGGACCGAGCGTCTTCCTCTTCCCACCCAA | |
| | GCCCAAGGATACCCTGATGATCTCCCGCACTCCCGAAGTGAC | |
| | CTGTGTGGTTGTGGACGTCTCCCATGAGGACCCTGAGGTTAA | |
| | GTTTAACTGGTACGTAGATGGCGTTGAGGTTCACAATGCCAA | |
| | GACCAAGCCGCGTGAAGAGCAGTATAACTCCACCTACAGGG | |
| | TCGTGAGCGTCCTGACCGTGCTTCATCAGGATTGGCTGAACG | |
| | GGAAGGAGTATAAGTGCAAAGTCAGCAACAAGGCCCTGCCC | |
| | GCACCTATCGAGAAGACCATTAGCAAGGCCAAGGGTCAGCC | |
| | TCGCGAGCCCCAGGTGTACACCCTGCCGCCCTCCCGTGACGA | |
| | GCTCACAAAGAACCAGGTGTCCTTGACCTGCCTGGTTAAAGG | |
| | ATTCTACCCGTCAGATATTGCCGTGGAATGGGAGAGCAATG | |
| | GCCAGCCTGAGAATAACTACAAGACAACTCCCCCGGTGCTC | |
| | GACTCCGACGGCAGTTTTTTCCTGTATTCAAAGCTCACAGTG | |
| | GATAAGTCTAGGTGGCAACAGGGTAATGTTTTCAGCTGTTCC | |
| | GTTATGCACGAGGCCCTCCACAACCACTACACCCAGAAATC | |
| | CCTGAGCCTGTCCCCAGGGAAG | |
| | Light Chain | |
| | LCDR1 (IMGT) | SEQ ID |
| | QSISSY | NO: 170 |
| | LCDR2 (IMGT) | SEQ ID |
| | GA | NO: 171 |
| | LCDR3 (IMGT) | SEQ ID |
| | QQVPFEPPT | NO: 172 |
| | LCDR1 (Kabat) | SEQ ID |
| | RASQSISSYLN | NO: 173 |
| | LCDR2 (Kabat) | SEQ ID |
| | GASSLQS | NO: 174 |
| | LCDR3 (Kabat) | SEQ ID |
| | QQVPFEPPT | NO: 175 |
| | LCDR1 (Combined Kabat/IMGT) | SEQ ID |
| | RASQSISSYLN | NO: 176 |
| | LCDR2 (Combined Kabat/IMGT) | SEQ ID |
| | GASSLQS | NO: 177 |
| | LCDR3 (Combined Kabat/IMGT) | SEQ ID |
| | QQVPFEPPT | NO: 178 |
| | VL-Amino acid sequence | SEQ ID |
| | DIQMTQSPSSLSASVGDRVTITCRASQSISSYLNWYQQKPGKAP | NO: 179 |
| | KLLIYGASSLQSGVPSRFSGSGSGTDFTLTISSLQPEDFATYYCQ | |
| | QVPFEPPTFGGGTKVEIK | |
| | VL-DNA sequence | SEQ ID |
| | GACATCCAGATGACCCAGTCTCCATCCTCCCTGTCTGCATCT | NO: 180 |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTACCGTTCGAGCCTCCTACT | |
| | TTTGGCGGAGGGACCAAGGTTGAGATCAAA | |
| | Light chain-Amino acid sequence | SEQ ID |
| | DIQMTQSPSSLSASVGDRVTITCRASQSISSYLNWYQQKPGKAP | NO: 181 |
| | KLLIYGASSLQSGVPSRFSGSGSGTDFTLTISSLQPEDFATYYCQ | |
| | QVPFEPPTFGGGTKVEIK | |
| | Light chain-DNA sequence | SEQ ID |
| | DIQMTQSPSSLSASVGDRVTITCRASQSISSYLNWYQQKPGKAP | NO: 182 |
| | KLLIYGASSLQSGVPSRFSGSGSGTDFTLTISSLQPEDFATYYCQ | |
| | QVPFEPPTFGGGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEC | |
| | GACATCCAGATGACCCAGTCTCCATCCTCCCTGTCTGCATCT | |
| | GTAGGAGACAGAGTCACCATCACTTGCCGGGCAAGTCAGAG | |
| | CATTAGCAGCTATTTAAATTGGTATCAGCAGAAACCAGGGA | |
| | AAGCCCCTAAGCTCCTGATCTATGGTGCATCCAGTTTGCAAA | |
| | GTGGGGTCCCATCAAGGTTCAGTGGCAGTGGATCTGGGACA | |
| | GATTTCACTCTCACCATCAGCAGTCTGCAACCTGAAGATTTT | |
| | GCAACTTACTACTGTCAGCAAGTACCGTTCGAGCCTCCTACT | |
| | TTTGGCGGAGGGACCAAGGTTGAGATCAAACGTACGGTGGC | |
| | TGCACCATCTGTCTTCATCTTCCCGCCATCTGATGAGCAGTTG | |
| | AAATCTGGAACTGCCTCTGTTGTGTGCCTGCTGAATAACTTC | |
| | TATCCCAGAGAGGCCAAAGTACAGTGGAAGGTGGATAACGC | |
| | CCTCCAATCGGGTAACTCCCAGGAGAGTGTCACAGAGCAGG | |
| | ACAGCAAGGACAGCACCTACAGCCTCAGCAGCACCCTGACG | |
| | CTGAGCAAAGCAGACTACGAGAAACACAAAGTCTACGCCTG | |
| | CGAAGTCACCCATCAGGGCCTGAGCTCGCCCGTCACAAAGA | |
| | GCTTCAACAGGGGAGAGTGT | |

### Antigen-Binding Fragments

The present disclosure, among other things, provides anti-HHLA2 antigen-binding fragments. As used herein, an “anti-HHLA2 antigen-binding fragment” comprises or is any protein or peptide-containing molecule comprising at least a portion of an immunoglobulin molecule containing at least one complementarity determining region (CDR) of a VH or a VL or an HHLA2 binding portion derived from any of the antibodies described herein. Antibody fragments can be obtained using conventional techniques known to those of skill in the art, and the fragments are screened for utility in the same manner as intact antibodies. Such functional antibody fragments can retain the ability to selectively bind with HHLA2.

Examples of anti-HHLA2 antigen-binding fragments described herein can include: (i) a Fab fragment, a monovalent fragment comprising VL, VH, CL, and CH1 domains; (ii) a F(ab′)2 fragment, a bivalent fragment comprising two Fab fragments linked by a disulfide bridge at a hinge region; (iii) a Fd fragment comprising VH and CH1 domains; (iv) a Fv fragment comprising VL and VH domains of a single arm of an antibody, (v) a diabody (dAb) fragment comprising a VH domain; (vi) a camelid or camelized variable domain; (vii) a scFv, a fusion protein of VH and VL regions; or (viii) a single domain antibody. In some embodiments, an anti-HHLA2 antigen-binding fragment thereof described herein comprises or is a heavy chain and a light chain (e.g., a half antibody).

### Methods of Making

The present disclosure, among other things, provides methods of making anti-HHLA2 antibodies or antigen-binding fragments thereof described herein. In some embodiments, an anti-HHLA2 antibody or an antigen-binding fragment thereof described herein is identified using a display technology, such as yeast display, phage display, or ribosome display. In some embodiments, an anti-HHLA2 antibody or an antigen-binding fragment thereof described herein is identified using a hybridoma library (e.g., a mammalian hybridoma library, e.g., a mouse hybridoma library), followed by supernatant screening.

Combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, each of which his hereby incorporated by reference in its entirety).

In some embodiments, an anti-HHLA2 antibody or antigen-binding fragment thereof described herein may be derived from other species. A humanized antibody is an antibody produced by recombinant DNA technology, in which some or all amino acids of a human immunoglobulin light chain or heavy chain that are not required for antigen binding (e.g., constant regions and/or framework regions of variable domains) are used to substitute for the corresponding amino acids from light chain or heavy chain of the cognate, nonhuman antibody. By way of example, a humanized version of a murine antibody to a given antigen has on both heavy and light chains: (1) constant regions of a human antibody; (2) FRs from the variable domains of a human antibody; and (3) CDRs from the murine antibody. Human FRs may be selected based on their highest sequence homology to mouse FR sequence. When necessary, one or more residues in human FRs can be changed to residues at corresponding positions in a murine antibody so as to preserve binding affinity of the humanized antibody to a target. This change is sometimes called “back mutation.” Similarly, forward mutations may be made to revert back to murine sequence for a desired reason, e.g. stability or affinity to a target. Humanized antibodies generally are less likely to elicit an immune response in humans as compared to chimeric human antibodies because the former contain considerably fewer non-human components.

Methods for humanizing non-human antibodies are well known in the art. Suitable methods for making humanized antibodies in accordance with the present disclosure are described in, e.g., Winter EP 0 239 400; Jones et al., Nature 321:522-525 (1986); Riechmann et al., Nature 332:323-327 (1988); Verhoeyen et al., Science 239: 1534-1536 (1988); Queen et al., Proc. Nat. Acad. ScL USA 86:10029 (1989); U.S. Pat. No. 6,180,370; and Orlandi et al., Proc. Natl. Acad. Sd. USA 86:3833 (1989); the disclosures of each of which are incorporated herein by reference in their entireties. Generally, transplantation of non-human (e.g., murine) CDRs onto a human antibody is achieved as follows. cDNAs encoding VH and VL are isolated from a hybridoma, and nucleic acid sequences encoding VH and VL including CDRs are determined by sequencing. Nucleic acid sequences encoding CDRs are inserted into corresponding regions of a human antibody VH or VL coding sequences and attached to human constant region gene segments of a desired isotype (e.g., γ1 for CH and κ for CL). Humanized heavy and light chain genes are co-expressed in mammalian host cells (e.g., CHO or NSO cells) to produce soluble humanized antibody. To facilitate large-scale production of antibodies, it is often desirable to select for a high expressor using, for example, a DHFR gene or GS gene in the producer line.

In some embodiments, an anti-HHLA2 antibody or antigen-binding fragment thereof described herein comprises or is a human antibody. Completely human antibodies may be particularly desirable for therapeutic treatment of human subjects. Human antibodies can be made by a variety of methods known in the art including phage display methods described above using antibody libraries derived from human immunoglobulin sequences (see, e.g., U.S. Pat. Nos. 4,444,887 and 4,716,111; and PCT publications WO 98/46645, WO 98/60433, WO 98/24893, WO 98/16664, WO 96/34096, WO 96/33735, and WO 91/10741; each of which is incorporated herein by reference in its entirety). Techniques are also available for the preparation of human monoclonal antibodies in, e.g., Cole et al., Monoclonal Antibodies and Cancer Therapy, Alan R. Riss, (1985); and Boerner et al., J. Immunol., 147(1):86-95, (1991), each of which is incorporated herein by reference in its entirety.

### Nucleic Acids

The present disclosure, among other things, provides nucleic acids encoding HHLA2 binding agents described herein (e.g., anti-HHLA2 antibodies or antigen-binding fragments thereof). The present disclosure includes nucleic acids encoding one or more heavy chains, VH domains, heavy chain FRs, heavy chain CDRs, heavy chain constant domains, light chains, VL domains, light chain FRs, light chain CDRs, light chain constant domains, or other immunoglobulin-like sequences, antibodies, or antigen-binding fragments thereof disclosed herein. Such nucleic acids may be present in a vector. Such nucleic acids may be present in the genome of a cell, e.g., a cell of a subject in need of treatment or a cell for production of an antibody, e.g. a mammalian cell for production of an anti-HHLA2 antibodies or antigen-binding fragments thereof described herein.

Nucleic acids encoding HHLA2 binding agents described herein (e.g., an anti-HHLA2 antibody or antigen-binding fragment thereof) may be modified to include codons that are optimized for expression in a particular cell type or organism. Codon optimized sequences are synthetic sequences, and preferably encode an identical polypeptide (or biologically active fragment of a full length polypeptide which has substantially the same activity as the full length polypeptide) encoded by a non-codon optimized parent polynucleotide. In some embodiments, a coding region of a nucleic acids encoding HHLA2 binding agents described herein, in whole or in part, may include an altered sequence to optimize codon usage for a particular cell type (e.g., a eukaryotic or prokaryotic cell). For example, a coding sequence for a humanized heavy (or light) chain variable region as described herein may be optimized for expression in a bacterial cells. Alternatively, the coding sequence may be optimized for expression in a mammalian cell (e.g., a CHO cell). Such a sequence may be described as a codon-optimized sequence.

Nucleic acid constructs of the present disclosure may be inserted into an expression vector or viral vector by methods known to the art, and nucleic acids may be operably linked to an expression control sequence. A vector comprising any nucleic acids or fragments thereof described herein is further provided by the present disclosure. Any nucleic acids or fragments thereof described herein can be cloned into any suitable vector and can be used to transform or transfect any suitable host. Selection of vectors and methods to construct them are commonly known to persons of ordinary skill in the art (see, e.g., “Recombinant DNA Part D,” Methods in Enzymology, Vol. 153, Wu and Grossman, eds., Academic Press (1987)).

Conventionally used techniques including, for example, electrophoresis, calcium phosphate precipitation, DEAE-dextran transfection, or lipofection, may be used to introduce a foreign nucleic acid (e.g., DNA or RNA) into a prokaryotic or eukaryotic host cell. Desirably, a vector may include regulatory sequences, such as transcription and/or translation initiation and/or termination codons, which are specific to the type of host (e.g., bacterium, fungus, plant, or animal) into which a vector is to be introduced, as appropriate and taking into consideration whether a vector is DNA or RNA. In some embodiments, a vector comprises regulatory sequences that are specific to a genus of a host cell. In some embodiments, a vector comprises regulatory sequences that are specific to a species of a host.

In addition to a replication system and an inserted nucleic acid, a nucleic acid construct can include one or more marker genes, which allow for selection of transformed or transfected hosts. Exemplary marker genes include, e.g., biocide resistance (e.g., resistance to antibiotics or heavy metals) or complementation in an auxotrophic host to provide prototrophy.

An expression vector can comprise a native or nonnative promoter operably linked to an isolated or purified nucleic acid as described above. Selection of promoters, e.g., strong, weak, inducible, tissue-specific, and/or developmental-specific, is within the skill of one in the art. Similarly, combining a nucleic acid as described above with a promoter is also within the skill of one in the art.

Suitable vectors include those designed for propagation and expansion and/or for expression. For example, a cloning vector may be selected from the pUC series, the pBluescript series (Stratagene, LaJolla, Calif), the pET series (Novagen, Madison, Wis.), the pGEX series (Pharmacia Biotech, Uppsala, Sweden), or the pEX series (Clontech, Palo Alto, Calif.). Bacteriophage vectors, such as λGT10, λGT11, λZapII (Stratagene), λEMBL4, and λNM1149, may be used. Examples of plant expression vectors that can be used include pBI110, pBI101.2, pBI101.3, pBIl21, or pBIN19 (Clontech). Examples of animal expression vectors that can be used include pEUK-C1, pMAM, or pMAMneo (Clontech). The TOPO cloning system (Invitrogen, Carlsbad, Calif.) also can be used in accordance with the manufacturer's recommendations.

Additional sequences can be added to such cloning and/or expression sequences to optimize their function in cloning and/or expression, to aid in isolation of a nucleic acid encoding an HHLA2 binding agent described herein, or to improve introduction of a nucleic acid into a cell. Use of cloning vectors, expression vectors, adapters, and linkers is well known in the art (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

In some embodiments, nucleic acids and vectors of the present disclosure are isolated and/or purified. The present disclosure also provides a composition comprising an isolated or purified nucleic acid, optionally in the form of a vector. Isolated nucleic acids and vectors may be prepared using standard techniques known in the art including, for example, alkali/SDS treatment, CsCl binding, column chromatography, agarose gel electrophoresis, and/or other techniques well known in the art. The composition can comprise other components as described further herein.

Any method known to one skilled in the art for the insertion of nucleic acids into a vector may be used to construct expression vectors encoding an anti-human HHLA2 antibody or antigen-binding fragment thereof described herein under control of transcriptional and/or translational control signals. These methods may include in vitro recombinant DNA and synthetic techniques and in vivo recombination (see, e.g., Ausubel, supra; or Sambrook, supra).

Antibodies that Bind to the Same Epitope

In some embodiments, anti-HHLA2 antibodies or antigen-binding fragments thereof described herein include antibodies and antibody fragments that bind to the same epitope as the HHLA2-binding antibodies shown in Table 1. Additional antibodies and antibody fragments can therefore be identified based on their ability to cross-compete (e.g., to competitively inhibit the binding of, in a statistically significant manner) with other antibodies described herein in HHLA2 binding assays. The ability of a test antibody to inhibit the binding of antibodies and antibody fragments described herein to a HHLA2 protein (e.g., human HHLA2) demonstrates that the test antibody can compete with that antibody or antibody fragment for binding to HHLA2; such an antibody may, according to non-limiting theory, bind to the same or a related (e.g., a structurally similar or spatially proximal) epitope on the HHLA2 protein as the antibody or antibody fragment with which it competes. In some embodiment, an antibody that binds to the same epitope on HHLA2 as an anti-HHLA2 antibody or antigen-binding fragment thereof described herein is a human or humanized monoclonal antibody. Such human or humanized monoclonal antibodies can be prepared and isolated as described herein.

### Methods of Treatment

The present disclosure, among other things, provides methods of treating a disease, disorder or condition (e.g., a disease, disorder or condition described herein) in a subject comprising administering a pharmaceutical composition comprising at least one HHLA2 binding agent described herein. In some embodiments, a therapeutically effective amount of at least one pharmaceutical composition described herein is administered to a subject having a disease, disorder, or condition.

Pharmaceutical compositions comprising at least one HHLA2 binding agent described herein can be for use in the manufacture of a medicament for treating a disease, disorder, or condition in a subject or stimulating an immune response in a subject. Pharmaceutical compositions at least one HHLA2 binding agent described herein can be administered to a subject in accordance with a dosage regimen described herein, alone or in combination with one or more therapeutic agents, procedures, or modalities.

A subject to be treated with methods described herein can be a mammal, e.g., a primate, e.g., a human (e.g., a patient having, or at risk of having, a disease, disorder or condition described herein). A method of treating (e.g., one or more of reducing, inhibiting, or delaying progression of) a cancer or a tumor in a subject with a pharmaceutical composition comprising at least one HHLA2 binding agent described herein is provided. A subject can have an adult or pediatric form of cancer. A cancer may be at an early, intermediate, or late stage, or a metastatic cancer.

A method of treating (e.g., one or more of reducing, inhibiting, or delaying progression of) a sign or symptom of cancer in a subject with a pharmaceutical composition comprising at least one HHLA2 binding agent described herein is provided. In some embodiments, pharmaceutical compositions described herein are useful to delay the onset of, slow the progression of, or ameliorate one or more signs or symptoms of cancer. In some embodiments, a physiological sign or symptom of cancer comprises or is an increase in tumor volume, an increase in number of cancer cells, an increase in number of metastases, a decrease in life expectancy, an increase in cancer cell proliferation, and/or an increase in cancer cell survival. In some embodiments, a physical sign or symptom of cancer comprises or is a skin lesion (e.g., a lump or mole), weight loss, digestive problems, discomfort, fatigue, pain, trouble swallowing, cough, unusual bleeding and/or discharge, changes in bowel and/or bladder habits, and/or mental confusion.

A cancer can include, but is not limited to, a solid tumor, a hematological cancer (e.g., leukemia, lymphoma, or myeloma, e.g., multiple myeloma), or a metastatic lesion. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma, e.g., a cutaneous melanoma), pancreas, and bones (e.g., a chordoma).

In some embodiments, a cancer is chosen from a lung cancer (e.g., a non-small cell lung cancer (NSCLC) (e.g., a non-small cell lung cancer (NSCLC) with squamous and/or non-squamous histology, or a NSCLC adenocarcinoma), or a small cell lung cancer (SCLC)), a skin cancer (e.g., a Merkel cell carcinoma or a melanoma (e.g., an advanced melanoma)), an ovarian cancer, a mesothelioma, a bladder cancer, a soft tissue sarcoma (e.g., a hemangiopericytoma (HPC)), a bone cancer (a bone sarcoma), a kidney cancer (e.g., a renal cancer (e.g., a renal cell carcinoma)), a liver cancer (e.g., a hepatocellular carcinoma), a cholangiocarcinoma, a sarcoma, a myelodysplastic syndrome (MDS), a prostate cancer, a breast cancer (e.g., a breast cancer that does not express one, two or all of estrogen receptor, progesterone receptor, or Her2/neu, e.g., a triple negative breast cancer), a colorectal cancer (e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer), a nasopharyngeal cancer, a duodenal cancer, an endometrial cancer, a pancreatic cancer, a head and neck cancer (e.g., head and neck squamous cell carcinoma (HNSCC)), an anal cancer, a gastro-esophageal cancer, a thyroid cancer (e.g., anaplastic thyroid carcinoma), a cervical cancer (e.g., a squamous cell carcinoma of the cervix), a neuroendocrine tumor (NET) (e.g., an atypical pulmonary carcinoid tumor), a lymphoproliferative disease (e.g., a post-transplant lymphoproliferative disease), a lymphoma (e.g., T-cell lymphoma, B-cell lymphoma, or a non-Hogdkin lymphoma), a myeloma (e.g., a multiple myeloma), or a leukemia (e.g., a myeloid leukemia or a lymphoid leukemia). In some embodiments, a subject has renal cell carcinoma.

In some embodiments, a cancer is a brain tumor, e.g., a glioblastoma, a gliosarcoma, or a recurrent brain tumor. In some embodiments, a cancer is a pancreatic cancer, e.g., an advanced pancreatic cancer. In some embodiments, a cancer is a skin cancer, e.g., a melanoma (e.g., a stage II-IV melanoma, an HLA-A2 positive melanoma, an unresectable melanoma, or a metastatic melanoma), or a Merkel cell carcinoma. In some embodiments, a cancer is a renal cancer, e.g., a renal cell carcinoma (RCC) (e.g., a metastatic renal cell carcinoma). In some embodiments, a cancer is a breast cancer, e.g., a metastatic breast carcinoma or a stage IV breast carcinoma, e.g., a triple negative breast cancer (TNBC). In some embodiments, a cancer is a virus-associated cancer. In some embodiments, a cancer is an anal canal cancer (e.g., a squamous cell carcinoma of the anal canal). In some embodiments, a cancer is a cervical cancer (e.g., a squamous cell carcinoma of the cervix). In some embodiments, a cancer is a gastric cancer (e.g., an Epstein Barr Virus (EBV) positive gastric cancer, or a gastric or gastro-esophageal junction carcinoma). In some embodiments, a cancer is a head and neck cancer (e.g., an HPV positive and negative squamous cell cancer of the head and neck (SCCHN)). In some embodiments, a cancer is a nasopharyngeal cancer (NPC). In some embodiments, a cancer is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer.

In some embodiments, a cancer is a hematological cancer. In some embodiments, a cancer is a leukemia, e.g., acute myeloid leukemia, chronic myeloid leukemia, acute lymphoblastic leukemia, chronic lymphocytic leukemia, chronic leukemia, or acute leukemia. In some embodiments, a cancer is a lymphoma, e.g., Hodgkin lymphoma (HL), non-Hodgkin's lymphoma, lymphocytic lymphoma, or diffuse large B cell lymphoma (DLBCL) (e.g., a relapsed or refractory HL or DLBCL). In some embodiments, a cancer is a myeloma, e.g., multiple myeloma.

Administration of pharmaceutical compositions at least one HHLA2 binding agent described herein may be carried out in any convenient manner (e.g., injection, ingestion, transfusion, inhalation, implantation, or transplantation). In some embodiments, a pharmaceutical compositions described herein is administered by injection or infusion. Pharmaceutical compositions described herein may be administered to a patient transarterially, subcutaneously, intravenously, intradermally, intratumorally, intranodally, intramedullary, intramuscularly, or intraperitoneally. In some embodiments, a pharmaceutical composition described herein is administered parenterally (e.g., intravenously, subcutaneously, intraperitoneally, or intramuscularly). In some embodiments, a pharmaceutical composition described herein is administered by subcutaneous, intravenous, intramuscular, or intrasternal infusion or injection. In some embodiments, a pharmaceutical composition described herein is administered by intramuscular or subcutaneous injection. Pharmaceutical compositions described herein may be injected directly into a site of inflammation, a local disease site, a lymph node, an organ, a tumor, or site of infection in a subject.

In some embodiments, at least one HHLA2 binding agent described herein is utilized in combination with one or more other therapeutic agents or modalities. In some embodiments, the one or more other therapeutic agents or modalities is also an anti-cancer agent or modality. In some embodiments the combination shows a synergistic effect in treating cancer. Known compounds or treatments that show therapeutic efficacy in treating cancer may include, for example, one or more chemotherapeutic agents, alkylating agents, anti-metabolites, anti-microtubule agents, topoisomerase inhibitors, cytotoxic antibiotics, angiogenesis inhibitors, immunomodulators, vaccines, cell-based therapies (e.g. allogeneic or autologous stem cell transplantation), organ transplantation, radiation therapy, and/or surgery.

### Pharmaceutical Compositions

The present disclosure, among other things, provides pharmaceutical compositions comprising at least one HHLA2 binding agent in combination with one or more pharmaceutically or physiologically acceptable carriers, diluents, or excipients.

When “a therapeutically effective amount, “an immunologically effective amount,” “an anti-immune response effective amount,” or “an immune response-inhibiting effective amount” is indicated, a precise amount of a pharmaceutical composition comprising at least one HHLA2 binding agent described herein can be determined by a physician with consideration of individual differences in age, weight, immune response, and condition of the patient (subject).

Pharmaceutical compositions described herein may comprise buffers including neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a pharmaceutical composition is substantially free of contaminants, e.g., there are no detectable levels of a contaminant (e.g., an endotoxin).

Pharmaceutical compositions described herein may be administered in a manner appropriate to the disease, disorder, or condition to be treated or prevented. Quantity and frequency of administration will be determined by such factors as condition of a patient, and type and severity of a patient's disease, disorder, or condition, although appropriate dosages may be determined by clinical trials.

Pharmaceutical compositions described herein may be in a variety of forms. These include, for example, liquid, semi-solid and solid dosage forms, such as liquid solutions (e.g., injectable and infusible solutions), dispersions or suspensions, liposomes, and suppositories. Preferred compositions may be injectable or infusible solutions. Pharmaceutical compositions described herein can be formulated for administration intravenously, subcutaneously, intradermally, intratumorally, intranodally, intramedullary, intramuscularly, transarterially, or intraperitoneally.

In some embodiments, a pharmaceutical composition described herein is formulated for parenteral (e.g., intravenous, subcutaneous, intraperitoneal, or intramuscular) administration. In some embodiments, a pharmaceutical composition described herein is formulated for subcutaneous, intravenous, intramuscular, or intrasternal injection or infusion. In preferred embodiments, a pharmaceutical composition described herein is formulated for subcutaneous or intravenous injection of infusion. Pharmaceutical compositions described herein can be formulated for administered by using infusion techniques that are commonly known in immunotherapy (See, e.g., Rosenberg et al., New Eng. J. of Med. 319:1676, 1988, which is hereby incorporated by reference in its entirety).

As used herein, the terms “parenteral administration” and “administered parenterally” refer to modes of administration other than enteral and topical administration, usually by injection or infusion, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intratumoral, and intrasternal injection and infusion.

In some embodiments, pharmaceutical compositions described herein are administered in combination with (e.g., before, simultaneously, or following) bone marrow transplantation or lymphocyte ablative therapy using a chemotherapy agent (e.g., fludarabine, external-beam radiation therapy (XRT), cyclophosphamide, or Rituxan). In certain embodiments, subjects undergo standard treatment with high dose chemotherapy followed by peripheral blood stem cell transplantation. In certain embodiments, following transplant, subjects receive one or more pharmaceutical compositions described herein. In some embodiments, pharmaceutical compositions described herein may be administered before or following surgery.

A dosage of any aforementioned therapy to be administered to a subject will vary with a disease, disorder, or condition being treated and based on a specific subject. Scaling of dosages for human administration can be performed according to art-accepted practices.

### Kits

The present disclosure, among other things, provides kits comprising at least one HHLA2 binding agent described herein, and instructions for use and/or administration. In some embodiments, a kit comprises least one HHLA2 binding agent described herein and a pharmaceutically acceptable carrier, and instructions for use and/or administration.

In some embodiments, a kit comprises instructions for use in any method described herein. Instructions can comprise a description of administration of the first and second pharmaceutical compositions to a subject to achieve the intended activity in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease, disorder or condition in a subject.

The kits provided herein are in suitable packaging. Suitable packaging includes, but is not limited to, vials, bottles, jars, flexible packaging, and the like. Also contemplated are packages for use in combination with a specific device, such as an infusion device. A kit may have a sterile access port (for example, the container may be an intravenous solution bag or a vial having a stopper pierce able by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### INCORPORATION BY REFERENCE

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following example. An example is provided for illustrative purposes only. It is not to be construed as limiting the scope or content of the disclosure in any way.

### EXAMPLE

The following example is provided so as to describe to the skilled artisan how to make and use methods and compositions described herein, and are not intended to limit the scope of the present disclosure.

### Example 1: Characterization of Certain HHLA2 Binding Agents

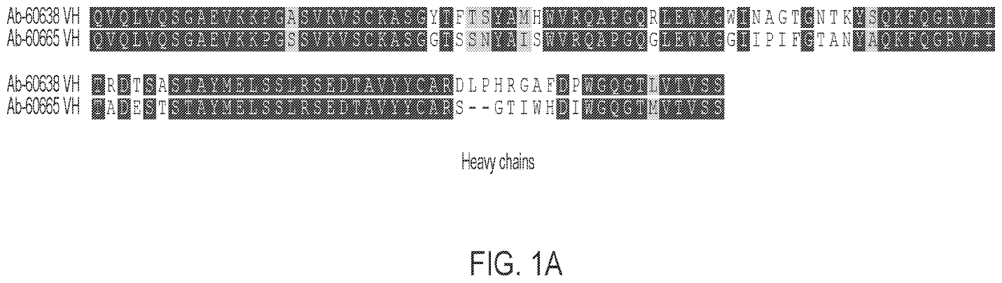

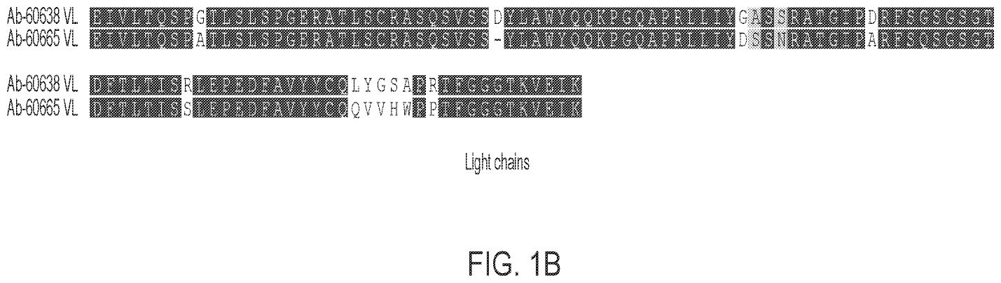

The present Example demonstrates methods for characterizing HHLA2 binding agents, specifically anti-HHLA2 antibodies and/or antigen-binding fragments thereof described herein. The present Example further provides various methods for determining and/or characterizing relevant functional activity of HHLA2 binding agents described herein. Alignments of heavy chain variable domains and light chain variable domain of exemplary anti-HHLA2 antibodies Ab-60638 and Ab-60665 described herein are shown in FIG. 1A-1B.

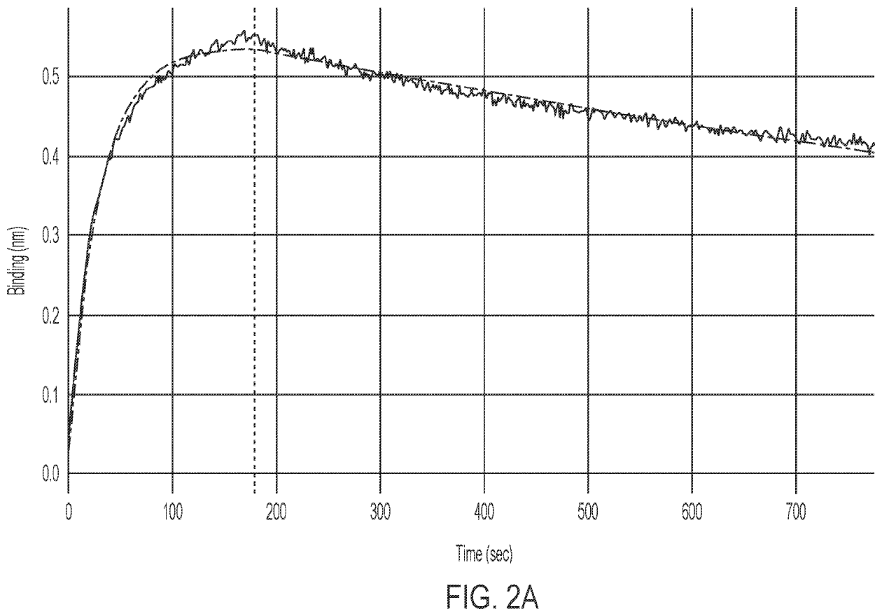

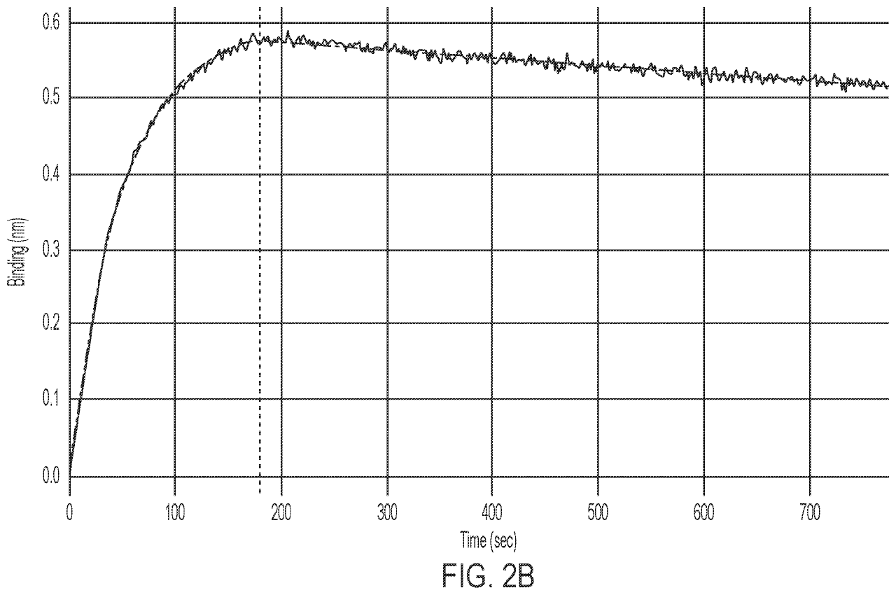

The avid affinity of the anti-HHLA2 antibodies for recombinant HHLA2-Fc was investigated. The Octet system from ForteBio was used to measure avid affinity of the anti-HHLA2 antibodies for recombinant HHLA2-Fc. Avid affinity was determined by capturing each antibody on an AHC sensor, followed by transfer to wells with 100 nM HHLA2-Fc antigen in solution. Avid affinities for exemplary anti-HHLA2 antibodies Ab-60638 and Ab-60665 showed single digit nanomolar avid affinities or better to recombinant HHLA2 (Table 2 and FIGS. 2A-2B).

TABLE 2
Avid affinity measurements for exemplary anti-
HHLA2 antibodies Ab-60638 and Ab-60665.

| Anti-HHLA2 | IgG K_{D} (M) | | |
|---|---|---|---|
| antibody | Avid | Kₒₙ (1/Ms) | K_{off} (1/s) |
| Ab-60638 | 1.42E−09 | 3.30E+05 | 4.69E−04 |
| Ab-60665 | 9.71E−10 | 1.94E+05 | 1.89E−04 |

The Biacore system from Cytiva was used to measure monovalent affinity of exemplary anti-HHLA2 antibodies Ab-65885, Ab-65886, Ab-65887, Ab-65889 and Ab-65890 for recombinant HHLA2-His. Monovalent affinity was determined by capturing each antibody on an CM5 chip, followed by various concentrations of the HHLA2-His analyte. Monovalent affinities for exemplary anti-HHLA2 antibodies Ab-65885, Ab-65886, Ab-65887, Ab-65889 and Ab-65890 to recombinant HHLA2 were about 750 pM to about 15 nM (Table 3).

TABLE 3
Monovalent affinity measurements for exemplary anti-HHLA2 antibodies
Ab-65885, Ab-65886, Ab-65887, Ab-65889 and Ab-65890.

| Anti-HHLA2 | IgG K_{D} (M) | | | |
|---|---|---|---|---|
| antibody | Monovalent | Kₒₙ (1/Ms) | K_{off} (1/s) | Rmax (RU) |
| Ab-65885 | 5.82E−09 | 3.25E+05 | 2.05E−03 | 77.76 |
| Ab-65886 | 1.13E−08 | 6.09E+05 | 6.88E−03 | 95.96 |
| Ab-65887 | 1.65E−08 | 9.00E+05 | 1.48E−02 | 63.73 |
| Ab-65889 | 8.94E−10 | 1.28E+05 | 1.14E−04 | 79.12 |
| Ab-65890 | 7.65E−10 | 1.23E+05 | 9.28E−05 | 71.49 |

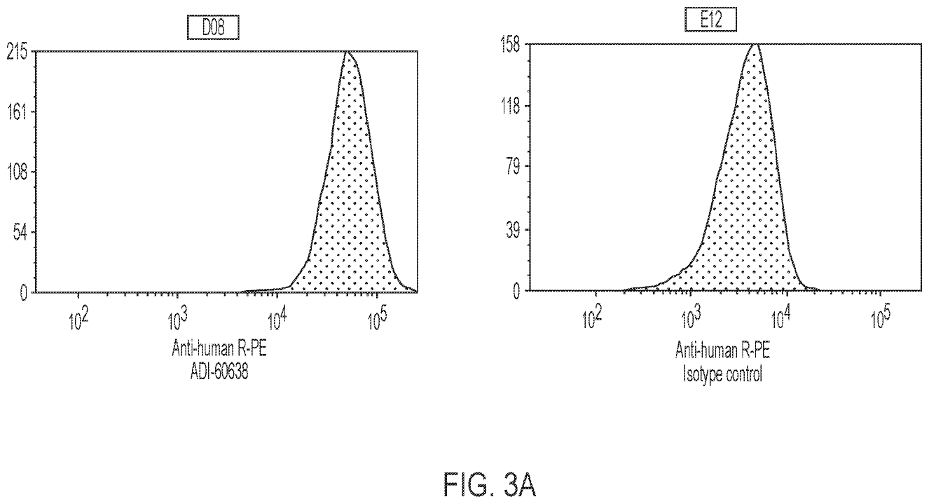

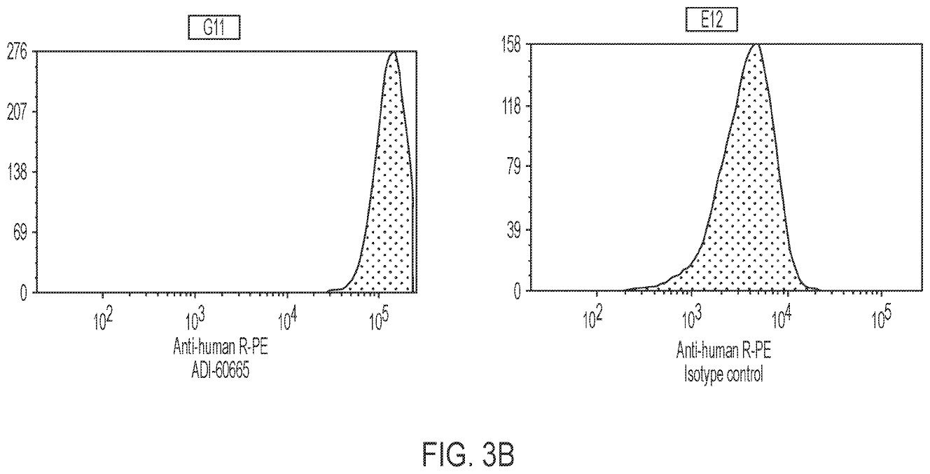

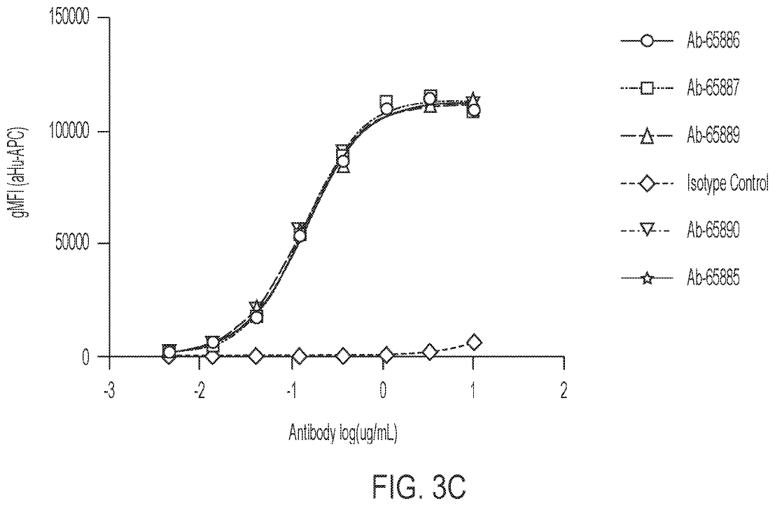

The ability of anti-HHLA2 antibodies to bind 300.19 mouse pre-B leukemic cells over-expressing human HHLA2 was investigated. 300.19-human HHLA2 cells were incubated with 15 μg/ml of each antibody for 30 minutes on ice, followed by a 1:10 dilution of a PE-conjugated anti-human secondary antibody. Flow cytometry histograms demonstrated the ability of exemplary anti-HHLA2 antibodies Ab-60638 and Ab-60665 to bind to 300.19 cells over-expressing human HHLA2 (Table 4 and FIGS. 3A-3B). Data are shown as median fluorescent intensity (MFI) and fold over background (FOB).

TABLE 4
Results from flow cytometry histograms for exemplary
anti-HHLA2 antibodies Ab-60638 and Ab-60665.

| | 300.19-human HHLA2 | 300.19-human HHLA2 |
|---|---|---|
| Anti-HHLA2 antibody | cells MFI | cells FOB |
| Ab-60638 | 46874 | 394 |
| Ab-60665 | 115373 | 820 |
| Isotype control | 3292 | 22 |

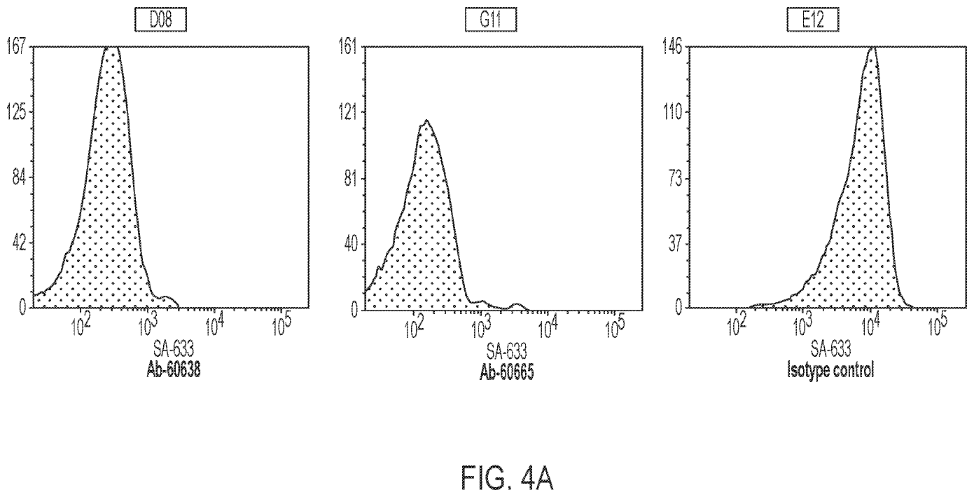

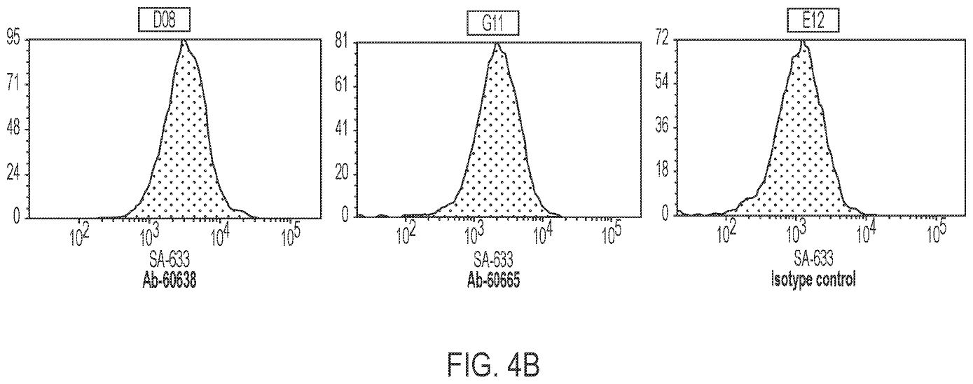

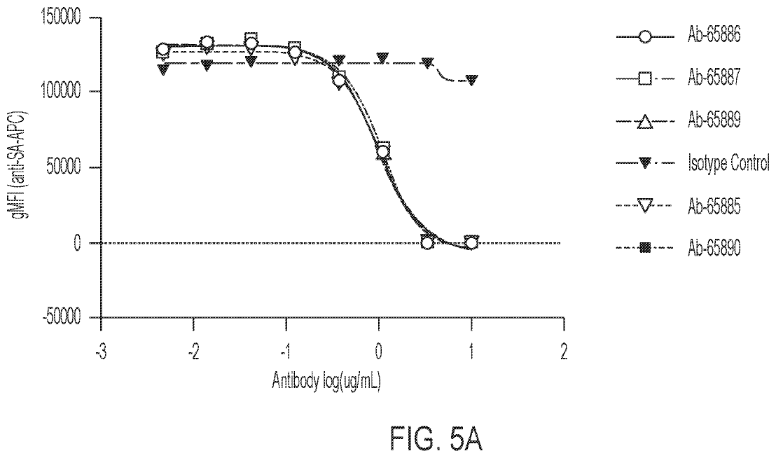

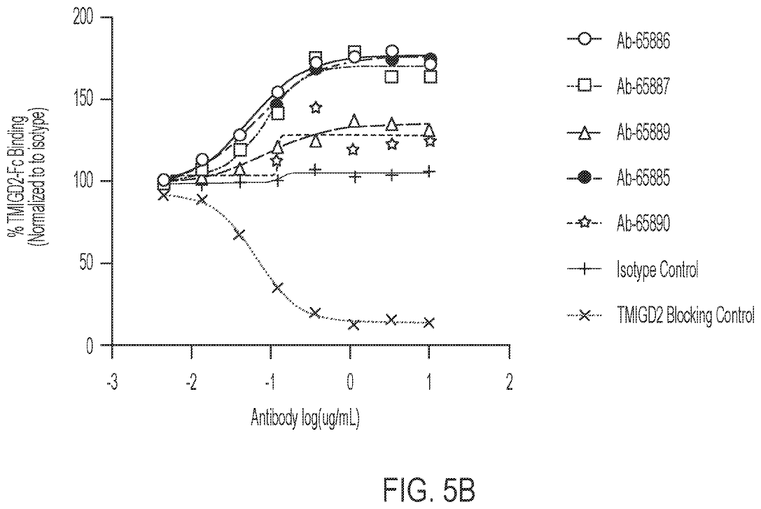

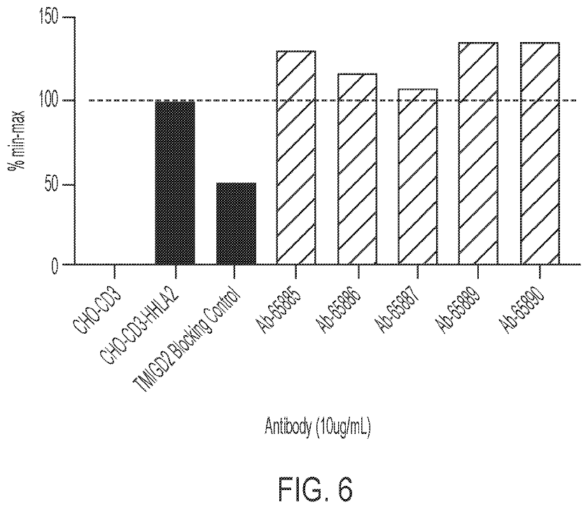

The ability of the anti-HHLA2 antibodies to block binding of HHLA2 to KIR3DL3 and enhance binding of HHLA2 to TMIGD2 was investigated. A total of 10 μg/mL IgG was incubated with 4 μg/mL biotinylated human HHLA2-Fc or TMIGD2-Fc on ice for 30 minutes then added to 300.19 mouse pre-B leukemic cells expressing KIR3DL3 or HHLA2, respectively, and continued incubation on ice for 30 minutes. Alexa Fluor 633 conjugated streptavidin was added as a secondary detection reagent. Flow cytometry data demonstrated the ability of the exemplary anti-HHLA2 antibodies to completely blocking HHLA2 binding to KIR3DL3, while increasing binding of HHLA2 to TMIGD2 (Table 5 and FIGS. 4A-4B and 5A-5B). These data show that the anti-HHLA2 antibodies described herein are capable of: (i) inhibiting HHLA2 binding to KIR3DL3; and (ii) enhancing HHLA2 binding to TMIGD2.

TABLE 5
Results from flow cytometry for exemplary anti-HHLA2 antibodies
Ab-60638 and Ab-60665 that block binding of HHLA2-Fc to
300.19 cells over-expressing KIR3DL3 and enhance binding
of TMIGD2-Fc to 300.19 cells over-expressing HHLA2.

| | Human | Antigen | | Antigen |
|---|---|---|---|---|
| | HHLA2-Fc | Binding | Human | Binding |
| | Binding MFI | MFI Ratio: | TMIGD2-Fc | MFI Ratio: |
| | for 300.19 | (IgG | Binding MFI | (IgG |
| Anti-HHLA2 | KIR3DL3 | blocked/ | for 300.19 | blocked/ |
| antibody | cells | antigen only) | HHLA2 cells | antigen only) |
| Ab-60638 | 201 | 0.0 | 2732 | 4.5 |
| Ab-60665 | 86 | 0.0 | 1859 | 3.1 |
| Isotype control | 6994 | 0.9 | 943 | 1.6 |

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes described herein. The publications, websites and other reference materials referenced herein to describe the background of the invention and to provide additional detail regarding its practice are hereby incorporated by reference in their entireties.

1. An HHLA2 binding agent capable of:
(i) inhibiting HHLA2 binding to KIR3DL3; and
(ii) enhancing HHLA2 binding to TMIGD2.

2. The HHLA2 binding agent of claim 1, wherein the HHLA2 binding agent is or comprises an antibody or antigen-binding fragment thereof, a small molecule, a polypeptide, or an aptamer.

3. The HHLA2 binding agent claim 2, wherein the antibody or antigen-binding fragment thereof is or comprises:
(i) a chimeric antibody, a human antibody, or a humanized antibody, or antigen-binding fragment thereof;
(ii) a monospecific antibody or a bispecific antibody, or antigen-binding fragment thereof;
(iii) a monoclonal antibody, or antigen-binding fragment thereof;
(iv) an scFv, Fab, Fab′, F(ab′)2, Fc, nanobody, or camelid antibody:
(v) a heavy chain constant region chosen from IgG1, IgG2, IgG3, or IgG4; and/or
(vi) a light chain constant region chosen from the light chain constant regions of kappa or lambda.

4.-5. (canceled)

6. The HHLA2 binding agent of claim 3, wherein the antibody or antigen-binding fragment thereof is or comprises:
(a) a heavy chain variable region (VH)
comprising a VH CDR1 amino acid sequence of SEQ ID NO: 105, a VH CDR2 amino acid sequence of SEQ ID NO: 106, and a VH CDR3 amino acid sequence of SEQ ID NO: 107;
(b) a light chain variable region (VL) comprising a VL CDR1 amino acid sequence of SEQ ID NO: 118, a VL CDR2 amino acid sequence of SEQ ID NO: 119, and a VL CDR3 amino acid sequence of SEQ ID NO: 120;
(c) a VH comprising a VH CDR1 amino acid sequence of SEQ ID NO: 108, a VH CDR2 amino acid sequence of SEQ ID NO: 109, and a VH CDR3 amino acid sequence of SEQ ID NO: 110;
(d) a VL comprising a VL CDR1 amino acid sequence of SEQ ID NO: 121, a VL CDR2 amino acid sequence of SEQ ID NO: 122, and a VL CDR3 amino acid sequence of SEQ ID NO: 123;
(e) a VH comprising a VH CDR1 amino acid sequence of SEQ ID NO: 111, a VH CDR2 amino acid sequence of SEQ ID NO: 112, and a VH CDR3 amino acid sequence of SEQ ID NO: 113; and/or
(f) a VL comprising a VL CDR1 amino acid sequence of SEQ ID NO: 124, a VL CDR2 amino acid sequence of SEQ ID NO: 125, and a VL CDR3 amino acid sequence of SEQ ID NO: 126.

7.-8. (canceled)

9. The HHLA2 binding agent of claim 6, wherein the antibody or antigen-binding fragment thereof is or comprises:
a VH with at least about 90% or more identity to SEQ ID NO: 114.

10. The HHLA2 binding agent of claim 9, wherein the antibody or antigen-binding fragment thereof is or comprises:
a VL with at least about 90% or more identity to SEQ ID NO: 127.

11. The HHLA2 binding agent of claim 10, wherein the antibody or antigen-binding fragment thereof is or comprises:
(a) a VH comprising SEQ ID NO: 114; and
(b) a VL comprising SEQ ID NO: 127.

12. The HHLA2 binding agent of claim 11, wherein the antibody or antigen-binding fragment thereof is or comprises:
a heavy chain with at least about 90% or more identity to SEQ ID NO: 116.

13. The HHLA2 binding agent of claim 11, wherein the antibody or antigen-binding fragment thereof is or comprises:
a light chain with at least about 90% or more identity to SEQ ID NO: 129.

14. The HHLA2 binding agent of claim 11, wherein the antibody or antigen-binding fragment thereof is or comprises:
(a) a heavy chain comprising SEQ ID NO: 116; and
(b) a light chain comprising SEQ ID NO: 129.

15. An agent that binds and/or competes for binding with the same epitope on HHLA2 as an HHLA2 binding agent of claim 1.

16. The HHLA2 binding agent of claim 1, wherein the binding agent:
enhances HHLA2 binding to TMIGD2 in naïve immune effector cells; and/or
blocks HHLA2 binding to KIR3DL3 in exhausted immune effector cells,
wherein the immune effector cells comprise or are T cells and/or NK cells,
wherein the T cells comprise or are CD4+ T cells and/or CD8+ T cells.

17.-19. (canceled)

20. The HHLA2 binding agent of claim 1, which:
binds HHLA2 with a K_{D} of about 15 nM or less;
binds human HHLA2 with an affinity of at least about 50-fold to about 800-fold over background; and/or
enhances HHLA2 binding to TMIGD2 at a ratio of greater than about 2.

21.-22. (canceled)

23. A pharmaceutical composition comprising at least one HHLA2 binding agent of claim 1, and a pharmaceutically acceptable carrier.

24. A method of treating a subject having a disease, disorder, or condition or modulating an immune response in a subject comprising:
administering a therapeutically effective amount of a pharmaceutical composition of claim 23 to the subject.

25.-26. (canceled)

27. The method of claim 24,
wherein the subject has a solid tumor or a hematological cancer;
wherein the solid tumor is or comprises one or more of:
a renal cancer, a bone cancer, a skin cancer, a breast cancer, a cervical cancer, a colorectal cancer, an endometrial cancer, a lung cancer, an ovarian cancer, a liver cancer, cholangiocarcinoma, or a thyroid cancer;
wherein the hematological cancer comprises or is a leukemia or lymphoma;
wherein the leukemia comprises or is acute lymphocytic leukemia, acute myeloid leukemia, chronic myeloid leukemia, acute lymphoblastic leukemia, chronic lymphocytic leukemia, chronic leukemia, or acute leukemia; or
wherein the lymphoma comprises or is Hodgkin lymphoma (HL), non-Hodgkin's lymphoma, lymphocytic lymphoma, or diffuse large B cell lymphoma (DLBCL).

28.-31. (canceled)

32. The method of claim 24, wherein the disease, disorder, or condition is associated with aberrant HHLA2 expression.

33. The method of claim 24, wherein the HHLA2 binding agent is administered parenterally.

34. The method of claim 33, wherein the parenteral administration is or comprises subcutaneous, intravenous, intramuscular, or intrasternal injection or infusion.

35. The method of claim 24, wherein the HHLA2 binding agent is administered in combination with a second agent.

36. A nucleic acid encoding at least one HHLA2 binding agent of claim 1.

37. An expression vector comprising at least one nucleic acid of claim 36.

38. A host cell comprising at least one expression vector of claim 37.

39. A method of making an HHLA2 binding agent, comprising:
(i) culturing a host cell of claim 38 under conditions suitable for expression of the HHLA2 binding agent, and
(ii) recovering the HHLA2 binding agent.

40. A method of detecting the presence or level of an HHLA2 polypeptide in a sample comprising:
detecting the HHLA2 polypeptide in the sample using at least one HHLA2 binding agent of claim 1.

41. A kit comprising at least one HHLA2 binding agent of claim 1, and instructions for use and/or administration.

42.-44. (canceled)


SEQUENCE LISTING (182 sequences)

SEQ ID NO: 1 - protein, 8 aa, Artificial Sequence
  Description of Artificial Sequence Synthetic peptide
GYTFTSYA

SEQ ID NO: 2 - protein, 8 aa, Artificial Sequence
  Description of Artificial Sequence Synthetic peptide
INAGTGNT

SEQ ID NO: 3 - protein, 12 aa, Artificial Sequence
  Description of Artificial Sequence Synthetic peptide
ARDLPHRGAFDP

SEQ ID NO: 4 - protein, 5 aa, Artificial Sequence
  Description of Artificial Sequence Synthetic peptide
SYAMH

SEQ ID NO: 5 - protein, 17 aa, Artificial Sequence
  Description of Artificial Sequence Synthetic peptide
WINAGTGNTKYSQKFQG

SEQ ID NO: 6 - protein, 10 aa, Artificial Sequence
  Description of Artificial Sequence Synthetic peptide
DLPHRGAFDP

SEQ ID NO: 7 - protein, 10 aa, Artificial Sequence
  Description of Artificial Sequence Synthetic peptide
GYTFTSYAMH

SEQ ID NO: 8 - protein, 17 aa, Artificial Sequence
  Description of Artificial Sequence Synthetic peptide
WINAGTGNTKYSQKFQG

SEQ ID NO: 9 - protein, 10 aa, Artificial Sequence
  Description of Artificial Sequence Synthetic peptide
DLPHRGAFDP

SEQ ID NO: 10 - protein, 119 aa, Artificial Sequence
  Description of Artificial Sequence Synthetic polypeptide
QVQLVQSGAEVKKPGASVKVSCKASGYTFTSYAMHWVRQAPGQRLEWMGWINAGTGNTKYSQKFQGRVTITRDTSASTAYMELSSLRSEDTAVYYCARDLPHRGAFDPWGQGTLVTVSS

SEQ ID NO: 11 - DNA, 357 bp, Artificial Sequence
  Description of Artificial Sequence Synthetic polynucleotide
caggtgcagctggtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcactggtaacacaaaatattcacaaaagttccagggcagagtcaccattaccagggacacatccgcgagcacagcctacatggagctgagcagcctgagatctgaagacacggcggtgtactactgcgccagagatcttcctcacagaggagcattcgacccatggggacagggtacattggtcaccgtctcctca

SEQ ID NO: 12 - protein, 446 aa, Artificial Sequence
  Description of Artificial Sequence Synthetic polypeptide
QVQLVQSGAEVKKPGASVKVSCKASGYTFTSYAMHWVRQAPGQRLEWMGWINAGTGNTKYSQKFQGRVTITRDTSASTAYMELSSLRSEDTAVYYCARDLPHRGAFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - DNA, 1338 bp, Artificial Sequence
  Description of Artificial Sequence Synthetic polynucleotide
caggtgcagctggtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcactggtaacacaaaatattcacaaaagttccagggcagagtcaccattaccagggacacatccgcgagcacagcctacatggagctgagcagcctgagatctgaagacacggcggtgtactactgcgccagagatcttcctcacagaggagcattcgacccatggggacagggtacattggtcaccgtctcctcagctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggcaaa

SEQ ID NO: 14 - protein, 7 aa, Artificial Sequence
  Description of Artificial Sequence Synthetic peptide
QSVSSDY

SEQ ID NO: 15 - protein, 3 aa, Artificial Sequence
  Description of Artificial Sequence Synthetic peptide
GAS

SEQ ID NO: 16 - protein, 9 aa, Artificial Sequence
  Description of Artificial Sequence Synthetic peptide
QLYGSAPRT

SEQ ID NO: 17 - protein, 12 aa, Artificial Sequence
  Description of Artificial Sequence Synthetic peptide
RASQSVSSDYLA

SEQ ID NO: 18 - protein, 7 aa, Artificial Sequence
  Description of Artificial Sequence Synthetic peptide
GASSRAT

SEQ ID NO: 19 - protein, 9 aa, Artificial Sequence
  Description of Artificial Sequence Synthetic peptide
QLYGSAPRT

SEQ ID NO: 20 - protein, 12 aa, Artificial Sequence
  Description of Artificial Sequence Synthetic peptide
RASQSVSSDYLA

SEQ ID NO: 21 - protein, 7 aa, Artificial Sequence
  Description of Artificial Sequence Synthetic peptide
GASSRAT

SEQ ID NO: 22 - protein, 9 aa, Artificial Sequence
  Description of Artificial Sequence Synthetic peptide
QLYGSAPRT

SEQ ID NO: 23 - protein, 108 aa, Artificial Sequence
  Description of Artificial Sequence Synthetic polypeptide
EIVLTQSPGTLSLSPGERATLSCRASQSVSSDYLAWYQQKPGQAPRLLIYGASSRATGIPDRFSGSGSGTDFTLTISRLEPEDFAVYYCQLYGSAPRTFGGGTKVEIK

SEQ ID NO: 24 - DNA, 324 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcagcgactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagctatacggaagtgcccctaggacttttggcggagggaccaaggttgagatcaaa

SEQ ID NO: 25 - protein, 215 aa, Artificial Sequence
  Description of Artificial Sequence Synthetic polypeptide
EIVLTQSPGTLSLSPGERATLSCRASQSVSSDYLAWYQQKPGQAPRLLIYGASSRATGIPDRFSGSGSGTDFTLTISRLEPEDFAVYYCQLYGSAPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 645 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcagcgactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagctatacggaagtgcccctaggacttttggcggagggaccaaggttgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 27 - protein, 8 aa, Artificial Sequence
  Description of Artificial Sequence Synthetic peptide
GGTSSNYA

SEQ ID NO: 28 - protein, 8 aa, Artificial Sequence
  Description of Artificial Sequence Synthetic peptide
IIPIFGTA

SEQ ID NO: 29 - protein, 10 aa, Artificial Sequence
  Description of Artificial Sequence Synthetic peptide
ARSGTIWHDI

SEQ ID NO: 30 - protein, 5 aa, Artificial Sequence
  Description of Artificial Sequence Synthetic peptide
NYAIS

SEQ ID NO: 31 - protein, 17 aa, Artificial Sequence
  Description of Artificial Sequence Synthetic peptide
GIIPIFGTANYAQKFQG

SEQ ID NO: 32 - protein, 8 aa, Artificial Sequence
  Description of Artificial Sequence Synthetic peptide
SGTIWHDI

SEQ ID NO: 33 - protein, 10 aa, Artificial Sequence
  Description of Artificial Sequence Synthetic peptide
GGTSSNYAIS

SEQ ID NO: 34 - protein, 17 aa, Artificial Sequence
  Description of Artificial Sequence Synthetic peptide
GIIPIFGTANYAQKFQG

SEQ ID NO: 35 - protein, 8 aa, Artificial Sequence
  Description of Artificial Sequence Synthetic peptide
SGTIWHDI

SEQ ID NO: 36 - protein, 117 aa, Artificial Sequence
  Description of Artificial Sequence Synthetic polypeptide
QVQLVQSGAEVKKPGSSVKVSCKASGGTSSNYAISWVRQAPGQGLEWMGGIIPIFGTANYAQKFQGRVTITADESTSTAYMELSSLRSEDTAVYYCARSGTIWHDIWGQGTMVTVSS

SEQ ID NO: 37 - DNA, 351 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcacctccagcaactatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagatcaggaacaatatggcacgacatatggggtcagggtacaatggtcaccgtctcctca

SEQ ID NO: 38 - protein, 444 aa, Artificial Sequence
  Description of Artificial Sequence Synthetic polypeptide
QVQLVQSGAEVKKPGSSVKVSCKASGGTSSNYAISWVRQAPGQGLEWMGGIIPIFGTANYAQKFQGRVTITADESTSTAYMELSSLRSEDTAVYYCARSGTIWHDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 1332 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcacctccagcaactatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggcggtgtactactgcgccagatcaggaacaatatggcacgacatatggggtcagggtacaatggtcaccgtctcctcagctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggcaaa

SEQ ID NO: 40 - protein, 6 aa, Artificial Sequence
  Description of Artificial Sequence Synthetic peptide
QSVSSY

SEQ ID NO: 41 - protein, 3 aa, Artificial Sequence
  Description of Artificial Sequence Synthetic peptide
DSS

SEQ ID NO: 42 - protein, 9 aa, Artificial Sequence
  Description of Artificial Sequence Synthetic peptide
QQVVHWPPT

SEQ ID NO: 43 - protein, 11 aa, Artificial Sequence
  Description of Artificial Sequence Synthetic peptide
RASQSVSSYLA

SEQ ID NO: 44 - protein, 7 aa, Artificial Sequence
  Description of Artificial Sequence Synthetic peptide
DSSNRAT

SEQ ID NO: 45 - protein, 9 aa, Artificial Sequence
  Description of Artificial Sequence Synthetic peptide
QQVVHWPPT

SEQ ID NO: 46 - protein, 11 aa, Artificial Sequence
  Description of Artificial Sequence Synthetic peptide
RASQSVSSYLA

SEQ ID NO: 47 - protein, 7 aa, Artificial Sequence
  Description of Artificial Sequence Synthetic peptide
DSSNRAT

SEQ ID NO: 48 - protein, 9 aa, Artificial Sequence
  Description of Artificial Sequence Synthetic peptide
QQVVHWPPT

SEQ ID NO: 49 - protein, 107 aa, Artificial Sequence
  Description of Artificial Sequence Synthetic polypeptide
EIVLTQSPATLSLSPGERATLSCRASQSVSSYLAWYQQKPGQAPRLLIYDSSNRATGIPARFSGSGSGTDFTLTISSLEPEDFAVYYCQQVVHWPPTFGGGTKVEIK

SEQ ID NO: 50 - DNA, 321 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgattcatccaacagggccactggcatcccagccaggttcagtggcagtgggtctgggacagacttcactctcaccatcagcagcctagagcctgaagattttgcagtttattactgtcagcaggtcgtccactggcctcccacttttggcggagggaccaaggttgagatcaaa

SEQ ID NO: 51 - protein, 214 aa, Artificial Sequence
  Description of Artificial Sequence Synthetic polypeptide
EIVLTQSPATLSLSPGERATLSCRASQSVSSYLAWYQQKPGQAPRLLIYDSSNRATGIPARFSGSGSGTDFTLTISSLEPEDFAVYYCQQVVHWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - DNA, 642 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgattcatccaacagggccactggcatcccagccaggttcagtggcagtgggtctgggacagacttcactctcaccatcagcagcctagagcctgaagattttgcagtttattactgtcagcaggtcgtccactggcctcccacttttggcggagggaccaaggttgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 53 - protein, 8 aa, Artificial Sequence
  Description of Artificial Sequence Synthetic peptide
GYTFDSYK

SEQ ID NO: 54 - protein, 8 aa, Artificial Sequence
  Description of Artificial Sequence Synthetic peptide
IPAGTGNT

SEQ ID NO: 55 - protein, 14 aa, Artificial Sequence
  Description of Artificial Sequence Synthetic peptide
ARDRGGYYYDSWDD

SEQ ID NO: 56 - protein, 5 aa, Artificial Sequence
  Description of Artificial Sequence Synthetic peptide
SYKMH

SEQ ID NO: 57 - protein, 17 aa, Artificial Sequence
  Description of Artificial Sequence Synthetic peptide
YIPAGTGNTKYSQKFQG

SEQ ID NO: 58 - protein, 12 aa, Artificial Sequence
  Description of Artificial Sequence Synthetic peptide
DRGGYYYDSWDD

SEQ ID NO: 59 - protein, 11 aa, Artificial Sequence
  Description of Artificial Sequence Synthetic peptide
SGYTFDSYKMH

SEQ ID NO: 60 - protein, 17 aa, Artificial Sequence
  Description of Artificial Sequence Synthetic peptide
YIPAGTGNTKYSQKFQG

SEQ ID NO: 61 - protein, 12 aa, Artificial Sequence
  Description of Artificial Sequence Synthetic peptide
DRGGYYYDSWDD

SEQ ID NO: 62 - protein, 121 aa, Artificial Sequence
  Description of Artificial Sequence Synthetic polypeptide
QVQLVQSGAEVKKPGASVKVSCKASGYTFDSYKMHWVRQAPGQRLEWMGYIPAGTGNTKYSQKFQGRVTITRDTSASTAYMELSSLRSEDTAVYYCARDRGGYYYDSWDDWGQGTLVTVSS

SEQ ID NO: 63 - DNA, 363 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcgacagctataaaatgcattgggtgcgccaggcccccggacaaaggcttgagtggatgggatacatccccgctggcactggtaacacaaaatattcacagaagttccagggcagagtcaccattaccagggacacatccgcgagcacagcctacatggagctgagcagcctgagatctgaagacacggcggtgtactactgcgccagagaccgtggcggatactactacgacagctgggatgattggggacagggtacattggtcaccgtctcctca

SEQ ID NO: 64 - protein, 451 aa, Artificial Sequence
  Description of Artificial Sequence Synthetic polypeptide
QVQLVQSGAEVKKPGASVKVSCKASGYTFDSYKMHWVRQAPGQRLEWMGYIPAGTGNTKYSQKFQGRVTITRDTSASTAYMELSSLRSEDTAVYYCARDRGGYYYDSWDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1353 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcgacagctataaaatgcattgggtgcgccaggcccccggacaaaggcttgagtggatgggatacatccccgctggcactggtaacacaaaatattcacagaagttccagggcagagtcaccattaccagggacacatccgcgagcacagcctacatggagctgagcagcctgagatctgaagacacggcggtgtactactgcgccagagaccgtggcggatactactacgacagctgggatgattggggacagggtacattggtcaccgtctcctcagcctccaccaagggcccttctgtgttccccctggcgccttccagtaagtccacctccggaggcacagccgctttgggctgtctggtcaaggactactttcctgagcccgtgaccgtctcctggaactcaggagccctgaccagtggggtacatacgtttccagccgtcttgcagtcctctggactgtactctctcagctctgtagtgaccgtgcccagcagttccctgggcacccagacatacatttgcaacgtcaatcacaagccgtctaacaccaaggtagataaaaaggtggagcctaagtcttgcgataagacccatacctgcccgccctgccccgccccagagctgctcgggggaccgagcgtcttcctcttcccacccaagcccaaggataccctgatgatctcccgcactcccgaagtgacctgtgtggttgtggacgtctcccatgaggaccctgaggttaagtttaactggtacgtagatggcgttgaggttcacaatgccaagaccaagccgcgtgaagagcagtataactccacctacagggtcgtgagcgtcctgaccgtgcttcatcaggattggctgaacgggaaggagtataagtgcaaagtcagcaacaaggccctgcccgcacctatcgagaagaccattagcaaggccaagggtcagcctcgcgagccccaggtgtacaccctgccgccctcccgtgacgagctcacaaagaaccaggtgtccttgacctgcctggttaaaggattctacccgtcagatattgccgtggaatgggagagcaatggccagcctgagaataactacaagacaactcccccggtgctcgactccgacggcagttttttcctgtattcaaagctcacagtggataagtctaggtggcaacagggtaatgttttcagctgttccgttatgcacgaggccctccacaaccactacacccagaaatccctgagcctgtccccagggaag

SEQ ID NO: 66 - protein, 6 aa, Artificial Sequence
  Description of Artificial Sequence Synthetic peptide
QSISSY

SEQ ID NO: 67 - protein, 2 aa, Artificial Sequence
  Description of Artificial Sequence Synthetic peptide
GA

SEQ ID NO: 68 - protein, 9 aa, Artificial Sequence
  Description of Artificial Sequence Synthetic peptide
QQVAFTPPT

SEQ ID NO: 69 - protein, 11 aa, Artificial Sequence
  Description of Artificial Sequence Synthetic peptide
RASQSISSYLN

SEQ ID NO: 70 - protein, 7 aa, Artificial Sequence
  Description of Artificial Sequence Synthetic peptide
GASSLQS

SEQ ID NO: 71 - protein, 9 aa, Artificial Sequence
  Description of Artificial Sequence Synthetic peptide
QQVAFTPPT

SEQ ID NO: 72 - protein, 11 aa, Artificial Sequence
  Description of Artificial Sequence Synthetic peptide
RASQSISSYLN

SEQ ID NO: 73 - protein, 7 aa, Artificial Sequence
  Description of Artificial Sequence Synthetic peptide
GASSLQS

SEQ ID NO: 74 - protein, 9 aa, Artificial Sequence
  Description of Artificial Sequence Synthetic peptide
QQVAFTPPT

SEQ ID NO: 75 - protein, 107 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQVAFTPPTFGGGTKVEIK

SEQ ID NO: 76 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcagcaagtagccttcactcctcctacttttggcggagggaccaaggttgagatcaaa

SEQ ID NO: 77 - protein, 214 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQVAFTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - DNA, 642 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcagcaagtagccttcactcctcctacttttggcggagggaccaaggttgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 79 - protein, 8 aa, Artificial Sequence
  Description of Artificial Sequence Synthetic peptide
GYTFEKYA

SEQ ID NO: 80 - protein, 8 aa, Artificial Sequence
  Description of Artificial Sequence Synthetic peptide
IPAGTGNT

SEQ ID NO: 81 - protein, 14 aa, Artificial Sequence
  Description of Artificial Sequence Synthetic peptide
ARDRGGYYYDSWDD

SEQ ID NO: 82 - protein, 5 aa, Artificial Sequence
  Description of Artificial Sequence Synthetic peptide
KYAMH

SEQ ID NO: 83 - protein, 17 aa, Artificial Sequence
  Description of Artificial Sequence Synthetic peptide
FIPAGTGNTKYSQKFQG

SEQ ID NO: 84 - protein, 12 aa, Artificial Sequence
  Description of Artificial Sequence Synthetic peptide
DRGGYYYDSWDD

SEQ ID NO: 85 - protein, 10 aa, Artificial Sequence
  Description of Artificial Sequence Synthetic peptide
GYTFEKYAMH

SEQ ID NO: 86 - protein, 17 aa, Artificial Sequence
  Description of Artificial Sequence Synthetic peptide
FIPAGTGNTKYSQKFQG

SEQ ID NO: 87 - protein, 12 aa, Artificial Sequence
  Description of Artificial Sequence Synthetic peptide
DRGGYYYDSWDD

SEQ ID NO: 88 - protein, 121 aa, Artificial Sequence
  Description of Artificial Sequence Synthetic polypeptide
QVQLVQSGAEVKKPGASVKVSCKASGYTFEKYAMHWVRQAPGQRLEWMGFIPAGTGNTKYSQKFQGRVTITRDTSASTAYMELSSLRSEDTAVYYCARDRGGYYYDSWDDWGQGTLVTVSS

SEQ ID NO: 89 - DNA, 363 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcgaaaaatatgctatgcattgggtgcgccaggcccccggacaaaggcttgagtggatgggattcatccccgctggcactggtaacacaaaatattcacagaagttccagggcagagtcaccattaccagggacacatccgcgagcacagcctacatggagctgagcagcctgagatctgaagacacggcggtgtactactgcgccagagaccgtggcggatactactacgacagctgggatgattggggacagggtacattggtcaccgtctcctca

SEQ ID NO: 90 - protein, 451 aa, Artificial Sequence
  Description of Artificial Sequence Synthetic polypeptide
QVQLVQSGAEVKKPGASVKVSCKASGYTFEKYAMHWVRQAPGQRLEWMGFIPAGTGNTKYSQKFQGRVTITRDTSASTAYMELSSLRSEDTAVYYCARDRGGYYYDSWDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1353 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcgaaaaatatgctatgcattgggtgcgccaggcccccggacaaaggcttgagtggatgggattcatccccgctggcactggtaacacaaaatattcacagaagttccagggcagagtcaccattaccagggacacatccgcgagcacagcctacatggagctgagcagcctgagatctgaagacacggcggtgtactactgcgccagagaccgtggcggatactactacgacagctgggatgattggggacagggtacattggtcaccgtctcctcagcctccaccaagggcccttctgtgttccccctggcgccttccagtaagtccacctccggaggcacagccgctttgggctgtctggtcaaggactactttcctgagcccgtgaccgtctcctggaactcaggagccctgaccagtggggtacatacgtttccagccgtcttgcagtcctctggactgtactctctcagctctgtagtgaccgtgcccagcagttccctgggcacccagacatacatttgcaacgtcaatcacaagccgtctaacaccaaggtagataaaaaggtggagcctaagtcttgcgataagacccatacctgcccgccctgccccgccccagagctgctcgggggaccgagcgtcttcctcttcccacccaagcccaaggataccctgatgatctcccgcactcccgaagtgacctgtgtggttgtggacgtctcccatgaggaccctgaggttaagtttaactggtacgtagatggcgttgaggttcacaatgccaagaccaagccgcgtgaagagcagtataactccacctacagggtcgtgagcgtcctgaccgtgcttcatcaggattggctgaacgggaaggagtataagtgcaaagtcagcaacaaggccctgcccgcacctatcgagaagaccattagcaaggccaagggtcagcctcgcgagccccaggtgtacaccctgccgccctcccgtgacgagctcacaaagaaccaggtgtccttgacctgcctggttaaaggattctacccgtcagatattgccgtggaatgggagagcaatggccagcctgagaataactacaagacaactcccccggtgctcgactccgacggcagttttttcctgtattcaaagctcacagtggataagtctaggtggcaacagggtaatgttttcagctgttccgttatgcacgaggccctccacaaccactacacccagaaatccctgagcctgtccccagggaag

SEQ ID NO: 92 - protein, 6 aa, Artificial Sequence
  Description of Artificial Sequence Synthetic peptide
QSISSY

SEQ ID NO: 93 - protein, 2 aa, Artificial Sequence
  Description of Artificial Sequence Synthetic peptide
GA

SEQ ID NO: 94 - protein, 9 aa, Artificial Sequence
  Description of Artificial Sequence Synthetic peptide
QQVAFTPPT

SEQ ID NO: 95 - protein, 11 aa, Artificial Sequence
  Description of Artificial Sequence Synthetic peptide
RASQSISSYLN

SEQ ID NO: 96 - protein, 7 aa, Artificial Sequence
  Description of Artificial Sequence Synthetic peptide
GASSLQS

SEQ ID NO: 97 - protein, 9 aa, Artificial Sequence
  Description of Artificial Sequence Synthetic peptide
QQVAFTPPT

SEQ ID NO: 98 - protein, 11 aa, Artificial Sequence
  Description of Artificial Sequence Synthetic peptide
RASQSISSYLN

SEQ ID NO: 99 - protein, 7 aa, Artificial Sequence
  Description of Artificial Sequence Synthetic peptide
GASSLQS

SEQ ID NO: 100 - protein, 9 aa, Artificial Sequence
  Description of Artificial Sequence Synthetic peptide
QQVAFTPPT

SEQ ID NO: 101 - protein, 107 aa, Artificial Sequence; identical to SEQ ID NO: 75
  Description of Artificial Sequence Synthetic polypeptide

SEQ ID NO: 102 - DNA, 321 bp, Artificial Sequence; identical to SEQ ID NO: 76
  Description of Artificial Sequence Synthetic polynucleotide

SEQ ID NO: 103 - protein, 214 aa, Artificial Sequence; identical to SEQ ID NO: 77
  Description of Artificial Sequence Synthetic polypeptide

SEQ ID NO: 104 - DNA, 642 bp, Artificial Sequence; identical to SEQ ID NO: 78
  Description of Artificial Sequence Synthetic polynucleotide

SEQ ID NO: 105 - protein, 8 aa, Artificial Sequence
  Description of Artificial Sequence Synthetic peptide
GYTFEQYA

SEQ ID NO: 106 - protein, 8 aa, Artificial Sequence
  Description of Artificial Sequence Synthetic peptide
IPAGTGNT

SEQ ID NO: 107 - protein, 14 aa, Artificial Sequence
  Description of Artificial Sequence Synthetic peptide
ARDRGGYWYDSWDD

SEQ ID NO: 108 - protein, 5 aa, Artificial Sequence
  Description of Artificial Sequence Synthetic peptide
QYAMH

SEQ ID NO: 109 - protein, 17 aa, Artificial Sequence
  Description of Artificial Sequence Synthetic peptide
FIPAGTGNTKYSQKFQG

SEQ ID NO: 110 - protein, 12 aa, Artificial Sequence
  Description of Artificial Sequence Synthetic peptide
DRGGYWYDSWDD

SEQ ID NO: 111 - protein, 10 aa, Artificial Sequence
  Description of Artificial Sequence Synthetic peptide
GYTFEQYAMH

SEQ ID NO: 112 - protein, 17 aa, Artificial Sequence
  Description of Artificial Sequence Synthetic peptide
FIPAGTGNTKYSQKFQG

SEQ ID NO: 113 - protein, 12 aa, Artificial Sequence
  Description of Artificial Sequence Synthetic peptide
DRGGYWYDSWDD

SEQ ID NO: 114 - protein, 121 aa, Artificial Sequence
  Description of Artificial Sequence Synthetic polypeptide
QVQLVQSGAEVKKPGASVKVSCKASGYTFEQYAMHWVRQAPGQRLEWMGFIPAGTGNTKYSQKFQGRVTITRDTSASTAYMELSSLRSEDTAVYYCARDRGGYWYDSWDDWGQGTLVTVSS

SEQ ID NO: 115 - DNA, 363 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcgaacaatatgctatgcattgggtgcgccaggcccccggacaaaggcttgagtggatgggattcatccccgctggcactggtaacacaaaatattcacagaagttccagggcagagtcaccattaccagggacacatccgcgagcacagcctacatggagctgagcagcctgagatctgaagacacggcggtgtactactgcgccagagaccgtggcggatactggtacgacagttgggatgattggggacagggtacattggtcaccgtctcctca

SEQ ID NO: 116 - protein, 451 aa, Artificial Sequence
  Description of Artificial Sequence Synthetic polypeptide
QVQLVQSGAEVKKPGASVKVSCKASGYTFEQYAMHWVRQAPGQRLEWMGFIPAGTGNTKYSQKFQGRVTITRDTSASTAYMELSSLRSEDTAVYYCARDRGGYWYDSWDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - DNA, 1353 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcgaacaatatgctatgcattgggtgcgccaggcccccggacaaaggcttgagtggatgggattcatccccgctggcactggtaacacaaaatattcacagaagttccagggcagagtcaccattaccagggacacatccgcgagcacagcctacatggagctgagcagcctgagatctgaagacacggcggtgtactactgcgccagagaccgtggcggatactggtacgacagttgggatgattggggacagggtacattggtcaccgtctcctcagcctccaccaagggcccttctgtgttccccctggcgccttccagtaagtccacctccggaggcacagccgctttgggctgtctggtcaaggactactttcctgagcccgtgaccgtctcctggaactcaggagccctgaccagtggggtacatacgtttccagccgtcttgcagtcctctggactgtactctctcagctctgtagtgaccgtgcccagcagttccctgggcacccagacatacatttgcaacgtcaatcacaagccgtctaacaccaaggtagataaaaaggtggagcctaagtcttgcgataagacccatacctgcccgccctgccccgccccagagctgctcgggggaccgagcgtcttcctcttcccacccaagcccaaggataccctgatgatctcccgcactcccgaagtgacctgtgtggttgtggacgtctcccatgaggaccctgaggttaagtttaactggtacgtagatggcgttgaggttcacaatgccaagaccaagccgcgtgaagagcagtataactccacctacagggtcgtgagcgtcctgaccgtgcttcatcaggattggctgaacgggaaggagtataagtgcaaagtcagcaacaaggccctgcccgcacctatcgagaagaccattagcaaggccaagggtcagcctcgcgagccccaggtgtacaccctgccgccctcccgtgacgagctcacaaagaaccaggtgtccttgacctgcctggttaaaggattctacccgtcagatattgccgtggaatgggagagcaatggccagcctgagaataactacaagacaactcccccggtgctcgactccgacggcagttttttcctgtattcaaagctcacagtggataagtctaggtggcaacagggtaatgttttcagctgttccgttatgcacgaggccctccacaaccactacacccagaaatccctgagcctgtccccagggaag

SEQ ID NO: 118 - protein, 6 aa, Artificial Sequence
  Description of Artificial Sequence Synthetic peptide
QSISSY

SEQ ID NO: 119 - protein, 2 aa, Artificial Sequence
  Description of Artificial Sequence Synthetic peptide
GA

SEQ ID NO: 120 - protein, 9 aa, Artificial Sequence
  Description of Artificial Sequence Synthetic peptide
QQVVFTPPT

SEQ ID NO: 121 - protein, 11 aa, Artificial Sequence
  Description of Artificial Sequence Synthetic peptide
RASQSISSYLN

SEQ ID NO: 122 - protein, 7 aa, Artificial Sequence
  Description of Artificial Sequence Synthetic peptide
GASSLQS

SEQ ID NO: 123 - protein, 9 aa, Artificial Sequence
  Description of Artificial Sequence Synthetic peptide
QQVVFTPPT

SEQ ID NO: 124 - protein, 11 aa, Artificial Sequence
  Description of Artificial Sequence Synthetic peptide
RASQSISSYLN

SEQ ID NO: 125 - protein, 7 aa, Artificial Sequence
  Description of Artificial Sequence Synthetic peptide
GASSLQS

SEQ ID NO: 126 - protein, 9 aa, Artificial Sequence
  Description of Artificial Sequence Synthetic peptide
QQVVFTPPT

SEQ ID NO: 127 - protein, 107 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQVVFTPPTFGGGTKVEIK

SEQ ID NO: 128 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcagcaagtagtgttcacgcctcctacttttggcggagggaccaaggttgagatcaaa

SEQ ID NO: 129 - protein, 214 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQVVFTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - DNA, 642 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcagcaagtagtgttcacgcctcctacttttggcggagggaccaaggttgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 131 - protein, 8 aa, Artificial Sequence
  Description of Artificial Sequence Synthetic peptide
GYTFASYK

SEQ ID NO: 132 - protein, 8 aa, Artificial Sequence
  Description of Artificial Sequence Synthetic peptide
IPAGTGNT

SEQ ID NO: 133 - protein, 14 aa, Artificial Sequence
  Description of Artificial Sequence Synthetic peptide
ARDRGGYYYDSWDD

SEQ ID NO: 134 - protein, 5 aa, Artificial Sequence
  Description of Artificial Sequence Synthetic peptide
SYKMH

SEQ ID NO: 135 - protein, 17 aa, Artificial Sequence
  Description of Artificial Sequence Synthetic peptide
DIPAGTGNTHYSQKFQG

SEQ ID NO: 136 - protein, 12 aa, Artificial Sequence
  Description of Artificial Sequence Synthetic peptide
DRGGYYYDSWDD

SEQ ID NO: 137 - protein, 10 aa, Artificial Sequence
  Description of Artificial Sequence Synthetic peptide
GYTFASYKMH

SEQ ID NO: 138 - protein, 17 aa, Artificial Sequence
  Description of Artificial Sequence Synthetic peptide
DIPAGTGNTHYSQKFQG

SEQ ID NO: 139 - protein, 12 aa, Artificial Sequence
  Description of Artificial Sequence Synthetic peptide
DRGGYYYDSWDD

SEQ ID NO: 140 - protein, 121 aa, Artificial Sequence
  Description of Artificial Sequence Synthetic polypeptide
QVQLVQSGAEVKKPGASVKVSCKASGYTFASYKMHWVRQAPGQRLEWMGDIPAGTGNTHYSQKFQGRVTITRDTSASTAYMELSSLRSEDTAVYYCARDRGGYYYDSWDDWGQGTLVTVSS

SEQ ID NO: 141 - DNA, 363 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcgccagctataaaatgcattgggtgcgccaggcccccggacaaaggcttgagtggatgggagacatccccgctggcactggtaacacacactattcacagaagttccagggcagagtcaccattaccagggacacatccgcgagcacagcctacatggagctgagcagcctgagatctgaagacacggcggtgtactactgcgccagagaccgtgggggatactactacgatagctgggatgattggggacagggtacattggtcaccgtctcctca

SEQ ID NO: 142 - protein, 451 aa, Artificial Sequence
  Description of Artificial Sequence Synthetic polypeptide
QVQLVQSGAEVKKPGASVKVSCKASGYTFASYKMHWVRQAPGQRLEWMGDIPAGTGNTHYSQKFQGRVTITRDTSASTAYMELSSLRSEDTAVYYCARDRGGYYYDSWDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - DNA, 1353 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcgccagctataaaatgcattgggtgcgccaggcccccggacaaaggcttgagtggatgggagacatccccgctggcactggtaacacacactattcacagaagttccagggcagagtcaccattaccagggacacatccgcgagcacagcctacatggagctgagcagcctgagatctgaagacacggcggtgtactactgcgccagagaccgtgggggatactactacgatagctgggatgattggggacagggtacattggtcaccgtctcctcagcctccaccaagggcccttctgtgttccccctggcgccttccagtaagtccacctccggaggcacagccgctttgggctgtctggtcaaggactactttcctgagcccgtgaccgtctcctggaactcaggagccctgaccagtggggtacatacgtttccagccgtcttgcagtcctctggactgtactctctcagctctgtagtgaccgtgcccagcagttccctgggcacccagacatacatttgcaacgtcaatcacaagccgtctaacaccaaggtagataaaaaggtggagcctaagtcttgcgataagacccatacctgcccgccctgccccgccccagagctgctcgggggaccgagcgtcttcctcttcccacccaagcccaaggataccctgatgatctcccgcactcccgaagtgacctgtgtggttgtggacgtctcccatgaggaccctgaggttaagtttaactggtacgtagatggcgttgaggttcacaatgccaagaccaagccgcgtgaagagcagtataactccacctacagggtcgtgagcgtcctgaccgtgcttcatcaggattggctgaacgggaaggagtataagtgcaaagtcagcaacaaggccctgcccgcacctatcgagaagaccattagcaaggccaagggtcagcctcgcgagccccaggtgtacaccctgccgccctcccgtgacgagctcacaaagaaccaggtgtccttgacctgcctggttaaaggattctacccgtcagatattgccgtggaatgggagagcaatggccagcctgagaataactacaagacaactcccccggtgctcgactccgacggcagttttttcctgtattcaaagctcacagtggataagtctaggtggcaacagggtaatgttttcagctgttccgttatgcacgaggccctccacaaccactacacccagaaatccctgagcctgtccccagggaag

SEQ ID NO: 144 - protein, 6 aa, Artificial Sequence
  Description of Artificial Sequence Synthetic peptide
QSISSY

SEQ ID NO: 145 - protein, 2 aa, Artificial Sequence
  Description of Artificial Sequence Synthetic peptide
GA

SEQ ID NO: 146 - protein, 9 aa, Artificial Sequence
  Description of Artificial Sequence Synthetic peptide
QQVPFEPPT

SEQ ID NO: 147 - protein, 11 aa, Artificial Sequence
  Description of Artificial Sequence Synthetic peptide
RASQSISSYLN

SEQ ID NO: 148 - protein, 7 aa, Artificial Sequence
  Description of Artificial Sequence Synthetic peptide
GASSLQS

SEQ ID NO: 149 - protein, 9 aa, Artificial Sequence
  Description of Artificial Sequence Synthetic peptide
QQVPFEPPT

SEQ ID NO: 150 - protein, 11 aa, Artificial Sequence
  Description of Artificial Sequence Synthetic peptide
RASQSISSYLN

SEQ ID NO: 151 - protein, 7 aa, Artificial Sequence
  Description of Artificial Sequence Synthetic peptide
GASSLQS

SEQ ID NO: 152 - protein, 9 aa, Artificial Sequence
  Description of Artificial Sequence Synthetic peptide
QQVPFEPPT

SEQ ID NO: 153 - protein, 107 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQVPFEPPTFGGGTKVEIK

SEQ ID NO: 154 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcagcaagtaccgttcgagcctcctacttttggcggagggaccaaggttgagatcaaa

SEQ ID NO: 155 - protein, 214 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQVPFEPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - DNA, 642 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcagcaagtaccgttcgagcctcctacttttggcggagggaccaaggttgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 157 - protein, 8 aa, Artificial Sequence
  Description of Artificial Sequence Synthetic peptide
GYTFNSYR

SEQ ID NO: 158 - protein, 8 aa, Artificial Sequence
  Description of Artificial Sequence Synthetic peptide
IPAGTGNT

SEQ ID NO: 159 - protein, 14 aa, Artificial Sequence
  Description of Artificial Sequence Synthetic peptide
ARDRGGYYYDSWDD

SEQ ID NO: 160 - protein, 5 aa, Artificial Sequence
  Description of Artificial Sequence Synthetic peptide
SYRMH

SEQ ID NO: 161 - protein, 17 aa, Artificial Sequence
  Description of Artificial Sequence Synthetic peptide
SIPAGTGNTVYSQKFQG

SEQ ID NO: 162 - protein, 12 aa, Artificial Sequence
  Description of Artificial Sequence Synthetic peptide
DRGGYYYDSWDD

SEQ ID NO: 163 - protein, 10 aa, Artificial Sequence
  Description of Artificial Sequence Synthetic peptide
GYTFNSYRMH

SEQ ID NO: 164 - protein, 17 aa, Artificial Sequence
  Description of Artificial Sequence Synthetic peptide
SIPAGTGNTVYSQKFQG

SEQ ID NO: 165 - protein, 12 aa, Artificial Sequence
  Description of Artificial Sequence Synthetic peptide
DRGGYYYDSWDD

SEQ ID NO: 166 - protein, 121 aa, Artificial Sequence
  Description of Artificial Sequence Synthetic polypeptide
QVQLVQSGAEVKKPGASVKVSCKASGYTFNSYRMHWVRQAPGQRLEWMGSIPAGTGNTVYSQKFQGRVTITRDTSASTAYMELSSLRSEDTAVYYCARDRGGYYYDSWDDWGQGTLVTVSS

SEQ ID NO: 167 - DNA, 363 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcaacagctatcgaatgcattgggtgcgccaggcccccggacaaaggcttgagtggatgggatccatccccgctggcactggtaacacagtctattcacagaagttccagggcagagtcaccattaccagggacacatccgcgagcacagcctacatggagctgagcagcctgagatctgaagacacggcggtgtactactgcgccagagacaggggcgggtactactacgacagctgggatgattggggacagggtacattggtcaccgtctcctca

SEQ ID NO: 168 - protein, 451 aa, Artificial Sequence
  Description of Artificial Sequence Synthetic polypeptide
QVQLVQSGAEVKKPGASVKVSCKASGYTFNSYRMHWVRQAPGQRLEWMGSIPAGTGNTVYSQKFQGRVTITRDTSASTAYMELSSLRSEDTAVYYCARDRGGYYYDSWDDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - DNA, 1353 bp, Artificial Sequence
  Description of Artificial Sequence Synthetic polynucleotide
caggtccagcttgtgcagtctggggctgaggtgaagaagcctggggcctcagtgaaggtttcctgcaaggcttctggatacaccttcaacagctatcgaatgcattgggtgcgccaggcccccggacaaaggcttgagtggatgggatccatccccgctggcactggtaacacagtctattcacagaagttccagggcagagtcaccattaccagggacacatccgcgagcacagcctacatggagctgagcagcctgagatctgaagacacggcggtgtactactgcgccagagacaggggcgggtactactacgacagctgggatgattggggacagggtacattggtcaccgtctcctcagcctccaccaagggcccttctgtgttccccctggcgccttccagtaagtccacctccggaggcacagccgctttgggctgtctggtcaaggactactttcctgagcccgtgaccgtctcctggaactcaggagccctgaccagtggggtacatacgtttccagccgtcttgcagtcctctggactgtactctctcagctctgtagtgaccgtgcccagcagttccctgggcacccagacatacatttgcaacgtcaatcacaagccgtctaacaccaaggtagataaaaaggtggagcctaagtcttgcgataagacccatacctgcccgccctgccccgccccagagctgctcgggggaccgagcgtcttcctcttcccacccaagcccaaggataccctgatgatctcccgcactcccgaagtgacctgtgtggttgtggacgtctcccatgaggaccctgaggttaagtttaactggtacgtagatggcgttgaggttcacaatgccaagaccaagccgcgtgaagagcagtataactccacctacagggtcgtgagcgtcctgaccgtgcttcatcaggattggctgaacgggaaggagtataagtgcaaagtcagcaacaaggccctgcccgcacctatcgagaagaccattagcaaggccaagggtcagcctcgcgagccccaggtgtacaccctgccgccctcccgtgacgagctcacaaagaaccaggtgtccttgacctgcctggttaaaggattctacccgtcagatattgccgtggaatgggagagcaatggccagcctgagaataactacaagacaactcccccggtgctcgactccgacggcagttttttcctgtattcaaagctcacagtggataagtctaggtggcaacagggtaatgttttcagctgttccgttatgcacgaggccctccacaaccactacacccagaaatccctgagcctgtccccagggaag

SEQ ID NO: 170 - protein, 6 aa, Artificial Sequence
  Description of Artificial Sequence Synthetic peptide
QSISSY

SEQ ID NO: 171 - protein, 2 aa, Artificial Sequence
  Description of Artificial Sequence Synthetic peptide
GA

SEQ ID NO: 172 - protein, 9 aa, Artificial Sequence
  Description of Artificial Sequence Synthetic peptide
QQVPFEPPT

SEQ ID NO: 173 - protein, 11 aa, Artificial Sequence
  Description of Artificial Sequence Synthetic peptide
RASQSISSYLN

SEQ ID NO: 174 - protein, 7 aa, Artificial Sequence
  Description of Artificial Sequence Synthetic peptide
GASSLQS

SEQ ID NO: 175 - protein, 9 aa, Artificial Sequence
  Description of Artificial Sequence Synthetic peptide
QQVPFEPPT

SEQ ID NO: 176 - protein, 11 aa, Artificial Sequence
  Description of Artificial Sequence Synthetic peptide
RASQSISSYLN

SEQ ID NO: 177 - protein, 7 aa, Artificial Sequence
  Description of Artificial Sequence Synthetic peptide
GASSLQS

SEQ ID NO: 178 - protein, 9 aa, Artificial Sequence
  Description of Artificial Sequence Synthetic peptide
QQVPFEPPT

SEQ ID NO: 179 - protein, 107 aa, Artificial Sequence; identical to SEQ ID NO: 153
  Description of Artificial Sequence Synthetic polypeptide

SEQ ID NO: 180 - DNA, 321 bp, Artificial Sequence; identical to SEQ ID NO: 154
  Description of Artificial Sequence Synthetic polynucleotide

SEQ ID NO: 181 - protein, 107 aa, Artificial Sequence; identical to SEQ ID NO: 153
  Description of Artificial Sequence Synthetic polypeptide

SEQ ID NO: 182 - DNA, 642 bp, Artificial Sequence; identical to SEQ ID NO: 156
  Description of Artificial Sequence Synthetic polynucleotide